(19) United States
(12) Patent Application Publication
(10) Pub. No.: US 2016/0250312 A1
(43) Pub. Date: Sep. 1, 2016

(54) MALARIA VACCINATION

(71) Applicants: ISIS INNOVATION LIMITED, Summertown, Oxford Oxfordshire (GB); ACADEMISCH ZIEKENHUIS LEIDEN (ALSO ACTING UNDER THE NAME OF LEIDEN UNIVERSITY MEDICAL), Leiden (ZA)

(72) Inventors: Rhea Longley, Oxford (GB); Ahmed Salman, Oxford (GB); Alexandra Spencer, Oxford (GB); Adrian Hill, Oxford (GB); Chris Janse, Leiden (NL); Sahid Khan, Leiden (NL)

(21) Appl. No.: 15/028,547

(22) PCT Filed: Oct. 13, 2014
(86) PCT No.: PCT/GB2014/053077

§ 371 Date: Apr. 11, 2016

(30) Foreign Application Priority Data
Oct. 11, 2013 (GB) 1318084.9

(51) Int. Cl. A61K 39/015; C12N 15/86; C12N 7/00; C07K 14/445; C07K 14/005

(52) U.S. Cl. CPC A61K 39/015; C07K 14/445; C07K 14/005; C12N 7/00; C12N 15/86; A61K 2039/53

(57) ABSTRACT

The invention relates to an antigenic composition or vaccine comprising a viral vector, the viral vector comprising nucleic acid encoding Plasmodium protein PfLSA1, or a part or variant of Plasmodium protein PfLSA1; PfLSAP2, or a part or variant of Plasmodium protein PfLSAP2; PfUIS3, or a part or variant of Plasmodium protein PfUIS3; PfI0580c, or a part or variant of Plasmodium protein PfI0580c; and PfSPECT-1, or a part or variant of Plasmodium protein PfSPECT-1.

This invention relates to antigenic compositions or vaccines comprising a viral vector for eliciting an immune response against Plasmodium infection, in particular for prevention or treatment of malaria.

Malaria is a serious and life-threatening mosquito-borne infectious disease caused by parasitic protozoans of the genus Plasmodium. Whilst preventative small molecule based medicines exist to prevent malaria, such as chloroquine, they can be associated with significant side-effects, they are unsuitable for long-term use, and drug resistance is increasingly problematic. Vaccination programs have been proven to be effective in reduction and eradication of various diseases worldwide. The aim is to develop an effective malaria vaccine, which is urgently needed. However, current single-component vaccines lack sufficient efficacy for deployment in the field. The two leading malaria vaccine candidates, RTS, S and ChAd63-MVA ME-TRAP, are both sub-unit vaccines targeting the pre-erythrocytic phase of malaria. Whilst neither vaccine currently provides optimal protective efficacy for deployment in endemic countries [1-4], they both demonstrate the strength of targeting the pre-erythrocytic phase, as no blood-stage vaccine has progressed as far in clinical development [5]. Vaccination with irradiated sporozoites delivered by mosquito bite has been considered the ‘gold-standard’ of malaria vaccines, as whilst it is impractical for deployment, this regimen has repeatedly shown sterile protection in vaccinated volunteers [6-12]. The increased efficacy of irradiated sporozoite immunization over sub-unit vaccines is likely because immune responses are induced to a broad range of antigenic targets. However, perhaps not only multiple targets are needed to create an efficacious sub-unit vaccine, but also better targets than those traditionally focused on (e.g. CSP and TRAP). Over 5000 different proteins are expressed throughout the Plasmodium life-cycle, leading to a high probability that a better target antigen than CSP or TRAP may exist, or a target antigen to be used along side CSP or TRAP in a multi-component vaccination strategy.

The problem with identifying suitable protective liver-stage antigens for use in a liver-stage vaccine is that there is no suitable small animal model of P.falciparum infection. There are rodent malaria models in mice but these are divergent from P. falciparum and many antigens in P. falciparum have no homologues in the rodent parasites. Furthermore hundreds or perhaps thousands of the 5000 or so genes in the P. falciparum genome are likely expressed in the liver and there has been no way of finding out which of these is a good vaccine antigen. However, it is likely that only a small number of the many genes expressed in the liver by P. falciparum produce proteins that end up as peptides presented by MHC class I molecules on the infected liver cell surface. These are the potential targets of vaccine-induced T cells whereas antigens the do not reach the surface in MHC molecules cannot be protective when using a liver-stage vaccine. Because parasite antigens in the liver are inside a parasitophorous vacuole, which is surrounded by a parasitophorous vacuole membrane most parasite antigens will be unable to reach the liver cell cytoplasm where they can be degraded, loaded on the MHC molecules and transported to the hepatocyte surface. Because it is not possible to identify the MHC-peptide complexes on the liver-cell surface directly, it has not been possible to determine which P. falciparum antigens can be a suitable liver-stage vaccine antigen.

LSA-1 was one of the first liver-stage proteins identified and one of the only known liver-stage specific proteins. LSA1 is well conserved amongst P. falciparum isolates [12], and is critical for late-liver stage development [13]. The likely function of PfLSA1 is in the transition from the liver-stage to the blood-stage, as it is expressed abundantly in the PV as flocculent material surrounding merozoites. It has been associated with protection in studies of natural immunity and in volunteers vaccinated with irradiated sporozoites [14-18]. A particularly strong association was found when HLA-B53-restricted cytotoxic T lymphocytes recognized a conserved epitope of PfLSA1, providing a molecular basis for the association of HLA-B53 with resistance to severe malaria in Africa [19]. A clinical trial of recombinant protein PfLSA1 administered with either of the adjuvants AS01 or AS02 (GSK) provided no protection against sporozoite challenge [20] probably because no CD8 T cells which could target the infected liver cell were induced in this trial. Another clinical trial of a polyprotein construct expressing six antigens including PfLSA1, delivered in a FP9-MVA prime-boost regimen, also demonstrated no efficacy and minimal immunogenicity [21]. Such failures highlight our historic inability to predict the effect of potentially promising vaccine candidates and the best method of delivery in order to provide protective immunity. A major challenge in identifying an immunogenic and protective liver-stage antigen has been the lack of a suitable pre-clinical assay.

Therefore, it would be desirable to provide alternative antigens, and improved delivery and vaccination methods for eliciting a protective immune response against malaria.

According to a first aspect of the invention, there is provided an antigenic composition or vaccine comprising a viral vector, the viral vector comprising nucleic acid encoding Plasmodium protein PfLSA1, or a part or variant of Plasmodium protein PfLSA1.

According to another aspect of the invention, there is provided an antigenic composition or vaccine comprising a viral vector, the viral vector comprising nucleic acid encoding Plasmodium protein PfLSAP2, or a part or variant of Plasmodium protein PfLSAP2.

According to another aspect of the invention, there is provided an antigenic composition or vaccine comprising a viral vector, the viral vector comprising nucleic acid encoding Plasmodium protein PfUIS3, or a part or variant of Plasmodium protein PfUIS3.

According to another aspect of the invention, there is provided an antigenic composition or vaccine comprising a viral vector, the viral vector comprising nucleic acid encoding Plasmodium protein PfI0580c, or a part or variant of Plasmodium protein PfI0580c.

According to another aspect of the invention, there is provided an antigenic composition or vaccine comprising a viral vector, the viral vector comprising nucleic acid encoding Plasmodium protein PfSPECT-1, or a part or variant of Plasmodium protein PfSPECT-1.

The antigenic composition or vaccine may be capable of eliciting a protective immune response against malaria in a subject.

Despite thousands of potential antigens being identified previously, including PfLSA1, the use of these antigens to provide a protective immune response has been unpredictable and has so far provided disappointing efficacy. The present invention has used new methodology to identify key candidate antigens that can be used in a viral vector delivery system to produce a protective immune response. The inventors have now devised a new solution to this problem that allows liver-stage antigens to be prioritised for inclusion in a liver-stage vaccine and even tested for efficacy in mice. In brief the method involves selection candidate antigens, expressing these in potent T cell inducing viral vectors, especially adenovirus and MVA vectors, and then inserting the gene for the same antigen into a transgenic Plasmodium berghei rodent parasite. These transgenic P. berghei parasite can then be used to test the efficacy of the viral vectored vaccine expressing the same antigen in mice. The results show a striking hierarchy of protective efficacy of leading candidate antigens with the surprising results that two antigens PfLSA-1 and LSAP2 show outstanding protective efficacy, PfUIS3 and PfI0580c show moderate protective efficacy and other leading antigens such as TRAP show little or no protective efficacy.

This work has led to the identification of PfLSA1 as an exceptionally promising antigen for a liver-stage vaccines, especially when expressed in viral vectors, such as adenoviral and MVA vectors.

The term “protective immune response” used herein, may be understood to be a host immune response that can sterilise the Plasmodium infection in a subject. The protective immune response may sterilise the Plasmodium infection in at least 25% of subjects treated. The protective immune response may sterilise the Plasmodium infection in at least 35% of subjects treated. The protective immune response may sterilise the Plasmodium infection in at least 40% of subjects treated. The protective immune response may sterilise the Plasmodium infection in at least 50% of subjects treated. The protective immune response may sterilise the Plasmodium infection in at least 60% of subjects treated. The protective immune response may provide clinical benefit in a subject by preventing the development of clinical malaria of a chronic parasitaemia. A protective immune response may comprise at least 0.2% of CD8+ T cells being antigen-specific as determined, for example, by flow cytometry staining, and/or at least 500 spot forming cells (SFU) per million peripheral blood mononuclear cells (PBMC). Spot forming cells (SFU) may be determined by an ELISpot assay (enzyme-linked immunsorbent spot assay (For example the ELISpot assay provided by Mabtech AB, Sweden, see: http://www.mabtech.com/Main/Page.asp?PageId=16). A protective immune response may comprise at least 0.1% of CD8+ T cells being antigen-specific. A protective immune response may comprise at least 0.4% of CD8+T cells being antigen-specific. A protective immune response may comprise at least 0.8% of CD8+ T cells being antigen-specific. A protective immune response may comprise at least 1% of CD8+ T cells being antigen-specific. A protective immune response may comprise at least 1000 spot forming cells (SFU) per million peripheral blood mononuclear cells (PBMC). A protective immune response may comprise at least 2000 spot forming cells (SFU) per million peripheral blood mononuclear cells (PBMC). A protective immune response may comprise at least 300 spot forming cells (SFU) per million peripheral blood mononuclear cells (PBMC). A protective immune response may comprise at least 100 spot forming cells (SFU) per million peripheral blood mononuclear cells (PBMC).

Where a nucleic acid sequence is provided, it is understood that the sequence may vary without changing the function via the use of redundant codons. For example one or more nucleotide bases or codons may be substituted with other nucleotide bases or codons, which still encode the same amino acid residue in a sequence. Where a peptide or protein sequence is provided, it is understood that the amino acid sequence may vary. For example, conservative amino acid substitutions may be provided to provide equal or similar function.

A viral vector may be a virus capable of delivering genetic material into a host cell, such as a mammalian host cell. The genetic material may be heterologous nucleic acid, which is not naturally encoded by the virus and/or the host cell. The viral vector may be modified by mutation to reduce its pathogenicity. The viral vector may be modified to encode and/or comprise an antigenic protein. The viral vector may comprise a adenovirus. The viral vector may comprise a Modified Vaccinia Ankara (MVA) virus. The viral vector may be selected from any of the group comprising, a poxvirus, such as Modified Vaccinia Ankara (MVA) virus, or an adenovirus. The adenovirus may comprise a simian adenovirus. The adenovirus may comprise a Group E adenovirus. The adenovirus may comprise ChAd63. The adenovirus may comprise ChAdOx1. The adenovirus may comprise a group A, B, C, D or E adenovirus. The adenovirus may comprise Ad35, Ad5, Ad6, Ad26, or Ad28. The adenovirus may be of simian (e.g. chimpanzee, gorilla or bonobo) origin. The adenovirus may comprise any of ChAd63, ChAdOx1, ChAdOx2, C6, C7, C9, PanAd3, or ChAd3. The composition may comprise two or more different viral vectors.

PfLSA1 may comprise or consist of the sequence of SEQ ID NO: 1 or SEQ ID NO: 2. The nucleic acid encoding PfLSA1 may comprise or consist of the sequence of SEQ ID NO: 3.

PfLSAP2 may comprise or consist of the sequence of SEQ ID NO: 4 or SEQ ID NO: 5. The nucleic acid encoding PfLSAP2 may comprise or consist of the sequence of SEQ ID NO: 6.

PfUIS3 may comprise or consist of the sequence of SEQ ID NO: 7. The nucleic acid encoding PfUIS3 may comprise or consist of the sequence of SEQ ID NO: 8.

PfI0580c may comprise or consist of the sequence of SEQ ID NO: 9 or 10. The nucleic acid encoding PfI0580c may comprise or consist of the sequence of SEQ ID NO: 11.

PfSPECT-1 may comprise or consist of the sequence of SEQ ID NO: 12 or SEQ ID NO: 13. The nucleic acid encoding PfSPECT-1 may comprise or consist of the sequence of SEQ ID NO: 14 or SEQ ID NO: 15.

Nucleic acid encoding the Plasmodium protein may be codon optimised. The codon optimisation may be for optimal translation in mammalian host cell, such as a human host cell.

A leader sequence, such as a tPA leader, may be encoded with the nucleic acid encoding the Plasmodium protein. The Plasmodium protein may be expressed with a tPA leader sequence. The Plasmodium protein may comprise a leader sequence, such as a tPA leader sequence.

The viral vector may comprise viral protein and a Plasmodium protein, or part thereof. The viral vector may comprise a virus particle comprising Plasmodium protein PfLSA1, or a part or variant of PfLSA1; and/or Plasmodium protein PfLSAP2, or a part or variant of PfLSAP2.

The Plasmodium may comprise P. falciparum. In particular, where LAS1 is the antigen, the Plasmodium may comprise P. falciparum. The Plasmodium may comprise P. vivax. The Plasmodium protein may be derived from P. falciparum. The Plasmodium protein may be derived from P. vivax. The malaria to be treated may comprise a P. falciparum infection. The malaria to be treated may comprise a P. vivax infection.

A “variant” of a Plasmodium protein may comprise an ortholog or homolog found in the same strain or species of Plasmodium, or found in a different strain or species of Plasmodium. For example, reference to a variant of PfLSAP2 may comprise the equivalent protein PFB0105c identified in P. vivax (Sargeant et al. Genome Biology 2006, 7:R12 (doi: 10.1186/gb-2006-7-2-r12; and Siau et al. PLoS Pathogens 2008. V.4, Issue 8). A variant may comprise a protein having one, two, three, four, five, six, seven, eight, nine, ten or more amino acid substitutions. The substitutions may be conservative substitutions. The amino acid substitutions may provide equivalent function. A “variant” of a Plasmodium protein may comprise a protein having a sequence identity of at least 60% with the Plasmodium protein. A “variant” of a Plasmodium protein may comprise a protein having a sequence identity of at least 65% with the Plasmodium protein. A “variant” of a Plasmodium protein may comprise a protein having a sequence identity of at least 70% with the Plasmodium protein. A “variant” of a Plasmodium protein may comprise a protein having a sequence identity of at least 80% with the Plasmodium protein. A “variant” of a Plasmodium protein may comprise a protein having a sequence identity of at least 90% with the Plasmodium protein. A “variant” of a Plasmodium protein may comprise a protein having a sequence identity of at least 95% with the Plasmodium protein. A “variant” of a Plasmodium protein may comprise a protein having a sequence identity of at least 98% with the Plasmodium protein. A “variant” of a Plasmodium protein may comprise a protein having a sequence identity of at least 99% with the Plasmodium protein.

A “part” of a Plasmodium protein may comprise a truncated version of the

Plasmodium protein. A “part” of a Plasmodium protein may comprise an antigenic section of the Plasmodium protein. For example, the epitope of the Plasmodium protein, which is recognised by the host immune response may be provided as part of the Plasmodium protein. A “part” of a Plasmodium protein may comprise at least 5 consecutive amino acids of the Plasmodium protein. A “part” of a Plasmodium protein may comprise at least 6, at least 7, at least 8, at least 9, at least 10, at least 11, at least 12, at least 13, at least 14, at least 15, at least 20, at least 30, or at least 50 consecutive amino acids of the Plasmodium protein.

The malaria may comprise liver-stage malaria. The malaria may comprise pre-erythrocytic-stage malaria. The malaria may comprise pre-erythrocytic-stage and/or blood-stage malaria.

The immunogenic composition or vaccine may be a multi-component/multi-antigen immunogenic composition or vaccine. The nucleic acid may further encode at least one other Plasmodium protein. The at least one other Plasmodium protein may be selected from the group comprising PfLSA1, PfLSAP2, PfUIS3, PfI0580c, PfSPECT-1, PfTRAP, PfCSP, PfRH5, PfAARP, Pfs25, Pfs230 PfAMA1, PfMSP1, and a Plasmodium antigen capable of eliciting an immunogenic response in a subject, or combinations thereof.

Where the immunogenic composition or vaccine is intended for a multiple administration regime, such as a prime-boost regime, the different administration may comprise identical or different immunogenic compositions or vaccines. Where the immunogenic composition or vaccine is intended for a prime-boost administration regime, the prime composition may comprise the same or different viral vector as the boost composition. The same immunogenic composition or vaccine may be used for both prime and boost administrations. A different immunogenic composition or vaccine may be used for the prime and boost administrations.

According to another aspect of the invention, there is provided a pharmaceutical composition comprising the immunogenic composition or vaccine according to the invention herein and a pharmaceutically acceptable carrier.

The pharmaceutically acceptable carrier may comprise saline, water, or buffer. The pharmaceutically acceptable carrier may comprise one or more compatible solid or liquid diluents or encapsulating substances which are suitable for administration to the body of a mammal, such as a human. The pharmaceutically acceptable carrier may be a liquid, solution, suspension, gel, ointment, lotion, powder, or combinations thereof. The pharmaceutically acceptable carrier may be a pharmaceutically acceptable aqueous carrier.

The pharmaceutical composition, immunogenic composition or vaccine may further comprise an adjuvant. The adjuvant may comprise an oil emulsion. The adjuvant may be selected from any of the group comprising PEI; Alum; AS01 or AS02 (GlaxoSmithKline); inorganic compounds, such as aluminum hydroxide, aluminum phosphate, calcium phosphate hydroxide, or beryllium; mineral oil, such as paraffin oil; emulsions, such as MF59; bacterial products, such as killed bacteria Bordetella pertussis, or Mycobacterium bovis; toxoids; non-bacterial organics, such as squalene or thimerosal; the saponin adjuvant matrix M (Isconova) or other ISCOM adjuvants; detergents, such as Quil A; cytokines, such as IL-1, IL-2, or IL-12; Freund's complete adjuvant; and Freund's incomplete adjuvant; or combinations thereof.

According to another aspect of the invention, there is provided a nucleic acid encoding a viral protein and a Plasmodium protein, wherein the Plasmodium protein comprises PfLSA1, or a part or variant of PfLSA1.

According to another aspect of the invention, there is provided a nucleic acid encoding a viral protein and a Plasmodium protein, wherein the Plasmodium protein comprises PfLSAP2, or a part or variant of PfLSAP2.

According to another aspect of the invention, there is provided a nucleic acid encoding a viral protein and a Plasmodium protein, wherein the Plasmodium protein comprises PfUIS3, or a part or variant of PfUIS3.

According to another aspect of the invention, there is provided a nucleic acid encoding a viral protein and a Plasmodium protein, wherein the Plasmodium protein comprises PfI0580c, or a part or variant of PfI0580c.

According to another aspect of the invention, there is provided a nucleic acid encoding a viral protein and a Plasmodium protein, wherein the Plasmodium protein comprises PfSPECT-1, or a part or variant of PfSPECT-1.

The nucleic acid may encode at least one additional Plasmodium protein, such as a Plasmodium protein selected from any of the group comprising PfLSA1, PfLSAP2, PfUIS3, PfI0580c, PfTRAP, PfCSP, PfSPECT-1, and a Plasmodium antigen capable of eliciting an immunogenic response in a subject, or combinations thereof.

According to another aspect of the invention, there is provided a nucleic acid encoding a viral protein and at least two Plasmodium proteins, wherein the Plasmodium proteins are selected from any of the group comprising PfLSA1, PfLSAP2, PfUIS3, PfI0580c, PfTRAP, PfCSP, PfSPECT-1, and a Plasmodium antigen capable of eliciting an immunogenic response in a subject, or a combination thereof.

The viral protein may comprise a simian adenoviral protein. The viral protein may comprise a Group E adenoviral protein. The viral protein may comprise a ChAd63 adenoviral protein. The viral protein may comprise a ChAdOx1 adenoviral protein. The viral protein may comprise an adenovirus protein or MVA virus protein.

According to another aspect of the invention, there is provided a virus comprising the nucleic acid according to the invention herein.

The virus particle may comprise Plasmodium protein PfLSA1, or a part or variant of PfLSA1. The virus particle may comprise Plasmodium protein PfLSAP2, or a part or variant of PfLSAP2. The virus particle may comprise Plasmodium protein PfUIS3, or a part or variant of PfUIS3. The virus particle may comprise Plasmodium protein PfI0580c, or a part or variant of PfI0580c. The virus particle may comprise Plasmodium protein PfSPECT-1, or a part or variant of PfSPECT-1. The virus particle may comprise at least one additional Plasmodium protein, such as a Plasmodium protein selected from any of the group comprising PfLSA1, PfLSAP2, PfUIS3, PfI0580c, PfTRAP, PfCSP, PfSPECT-1, and a Plasmodium antigen capable of eliciting an immunogenic response in a subject, or combinations thereof. The virus particle may comprise at least two Plasmodium proteins selected from any of the group comprising PfLSA1, PfLSAP2, PfUIS3, PfI0580c, PfTRAP, PfCSP, PfSPECT-1, and a Plasmodium antigen capable of eliciting an immunogenic response in a subject, or a combination thereof.

The virus may comprise adenovirus or MVA. The virus may comprise a simian adenovirus. The virus may comprise a Group E adenovirus. The virus may comprise ChAd63. The virus may comprise ChAdOx1.

According to another aspect of the invention, there is provided a host cell comprising the nucleic acid according to the invention herein.

The host cell may be in vitro. The host cell may be infected with the virus of the invention herein.

According to another aspect of the invention, there is provided a method of eliciting a protective immune response to a protein of Plasmodium in a host, comprising administering the pharmaceutical composition, the immunogenic composition or vaccine according to the invention herein.

The protective immune response may be a CD8+ T-cell response and/or a humoral response. The protective immune response may comprise at least 0.2% of CD8+ T cells being antigen-specific as determined, for example, by flow cytometry staining, and/or at least 500 spot forming cells (SFU) per million peripheral blood mononuclear cells (PBMC). Spot forming cells (SFU) may be determined by an ELISpot assay (enzyme-linked immunsorbent spot assay (For example the ELISpot assay provided by Mabtech AB, Sweden, see: http:///www.mabtech.com/Main/Page.asp?PageId=1 6).

According to another aspect of the invention, there is provided a method of prevention or treatment of malaria in a subject, comprising the administration of the pharmaceutical composition, the immunogenic composition or vaccine according to the invention herein.

The administered may be a single dose vaccination regime. The administered may be a single dose vaccination regime using just the adenoviral vector, or the MVA vector, or a mixture of both. The administered may be part of a prime-boost vaccination regime in a subject, where a first/prime administration of the pharmaceutical composition, the immunogenic composition or vaccine according to the invention is followed by a second/boost administration of the pharmaceutical composition, the immunogenic composition or vaccine according to the invention. Additional boost vaccinations may be provided.

The viral vector of the first/prime administration may comprise adenovirus. The viral vector of the second/boost administration may comprise poxvirus, such as MVA, or adenovirus.

According to another aspect of the invention, there is provided a method of prevention or treatment of malaria in a subject, comprising:
a first administration of the pharmaceutical composition, the immunogenic composition or vaccine according to the invention herein; and
a second administration of the pharmaceutical composition, the immunogenic composition or vaccine according to the invention herein.

The second/boost administration may be between about 7 days and about 30 days after the first/prime administration. The second/boost administration may be about 14 days after the first/prime administration.

Additional administrations of the pharmaceutical composition, the immunogenic composition or vaccine according to the invention herein may be provided.

According to another aspect of the invention, there is provided the pharmaceutical composition, the immunogenic composition or vaccine according to the invention herein, for use in prevention or treatment of malaria in a subject.

The use may be in a single dose vaccination regime in a subject. The use may be in a prime-boost vaccination regime in the subject.

According to another aspect of the invention, there is provided a kit for a vaccination regime against malaria in a subject, comprising:
a prime composition comprising a viral vector comprising nucleic acid encoding Plasmodium protein PfLSA1, or a part or variant of Plasmodium protein PfLSA1;
a boost composition comprising a viral vector comprising nucleic acid encoding Plasmodium protein PfLSA1, or a part or variant of Plasmodium protein PfLSA1.

According to another aspect of the invention, there is provided a kit for a vaccination regime against malaria in a subject, comprising:
a prime composition comprising a viral vector comprising nucleic acid encoding Plasmodium protein PfLSAP2, or a part or variant of Plasmodium protein PfLSAP2;
a boost composition comprising a viral vector comprising nucleic acid encoding Plasmodium protein PfLSAP2, or a part or variant of Plasmodium protein PfLSAP2.

According to another aspect of the invention, there is provided a kit for a vaccination regime against malaria in a subject, comprising:
a prime composition comprising a viral vector comprising nucleic acid encoding Plasmodium protein PfUIS3, or a part or variant of Plasmodium protein PfUIS3;
a boost composition comprising a viral vector comprising nucleic acid encoding Plasmodium protein PfUIS3, or a part or variant of Plasmodium protein PfUIS3.

According to another aspect of the invention, there is provided a kit for a vaccination regime against malaria in a subject, comprising:
a prime composition comprising a viral vector comprising nucleic acid encoding Plasmodium protein PfI0580c, or a part or variant of Plasmodium protein PfI0580c;
a boost composition comprising a viral vector comprising nucleic acid encoding Plasmodium protein PfI0580c, or a part or variant of Plasmodium protein PfI0580c.

According to another aspect of the invention, there is provided a kit for a vaccination regime against malaria in a subject, comprising:
a prime composition comprising a viral vector comprising nucleic acid encoding Plasmodium protein PfSPECT-1, or a part or variant of Plasmodium protein PfSPECT-1;
a boost composition comprising a viral vector comprising nucleic acid encoding Plasmodium protein PfSPECT-1, or a part or variant of Plasmodium protein PfSPECT-1.

The kit may further comprise directions to administer the prime composition prior to the boost composition in a subject. The nucleic acid of the viral vector of the kit may further encode one or more other Plasmodium proteins. The one or more other Plasmodium proteins may comprise Plasmodium antigens capable of eliciting an immune response in a subject. The one or more other Plasmodium proteins may comprise PfLSA1, PfLSAP2, PfUIS3, PfI0580c, PfTRAP, PfCSP, PfSPECT-1, or a Plasmodium antigen capable of eliciting an immunogenic response in a subject, or combinations thereof.

The kit, or prime and/or boost composition may further comprise an adjuvant.

According to another aspect of the invention, there is provided a method of manufacturing an immunogenic composition or vaccine according to the invention herein, comprising:
culturing host cells capable of facilitating viral replication;
infecting the host cells with a virus according to the invention herein, or transforming the cells with nucleic acid according to the invention herein;
incubating the host cells to allow the production of viral progeny; and
harvesting the viral progeny to provide the immunogenic composition or vaccine.

In aspects and embodiments of the invention, the Plasmodium gene encoding the antigenic protein of the invention may be under control of the regulatory regions (e.g. the promoter and transcriptional terminator sequences) of the P. berghei UIS4 gene. The viral vector or nucleic acid of the invention herein may comprise the promoter and transcriptional terminator sequences) of the P. berghei UIS4 gene.

The skilled person will understand that optional features of one embodiment or aspect of the invention may be applicable, where appropriate, to other embodiments or aspects of the invention.

There now follows by way of example only a detailed description of the present invention with reference to the accompanying drawings, in which;

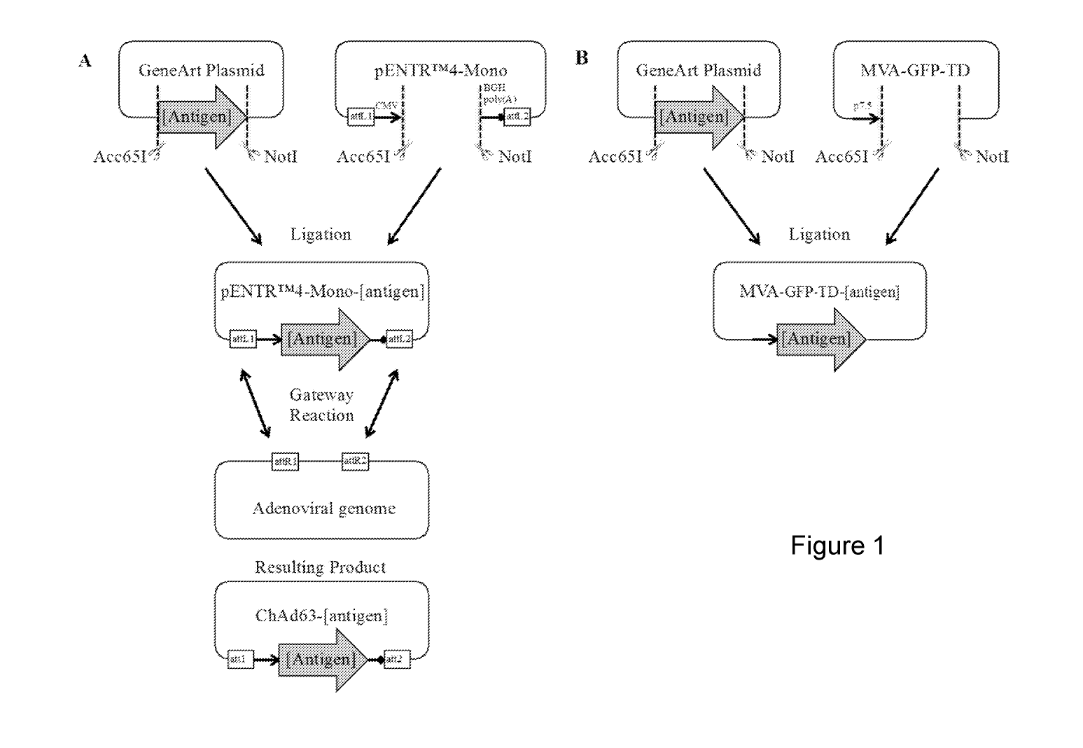

FIG. 1: Cloning scheme for insertion of liver-stage malaria antigens into the viral vectors ChAd63 and MVA.(A) To create ChAd63-[antigen] vaccines, the antigen of interest was first cloned into the entry vector pENTR™ 4-Mono by ligation, after digestion with the restriction enzymes Acc651I and NotI. The entry vector was then inserted into the ChAd63 genome through site-specific recombination using the Gateway® method. (B) To create MVA-[antigen] vaccines, a one-step cloning method was used. The antigen of interest was cloned into the markerless MVA genome by ligation, after digestion with the restriction enzymes Acc651T and NotI.

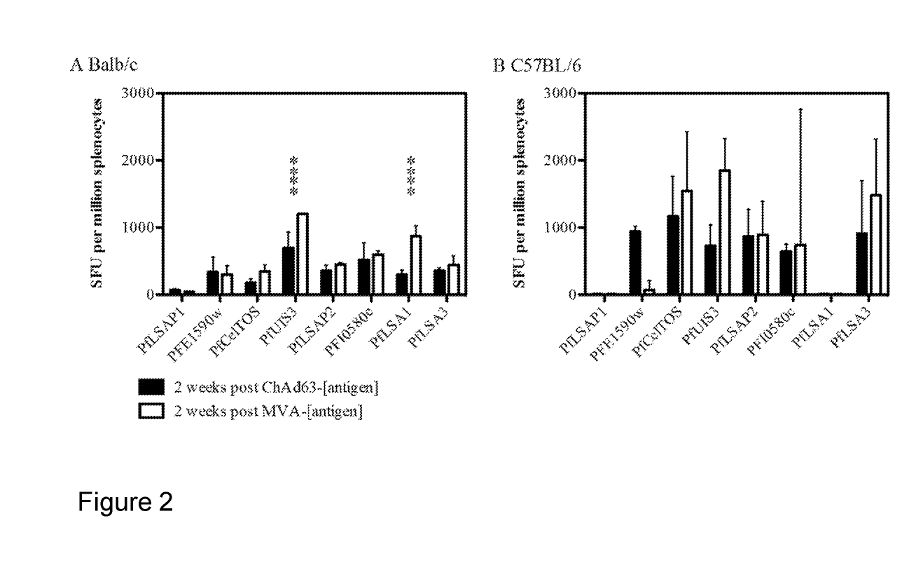

FIG. 2: Cellular immunogenicity of the eight candidate P. falciparum vaccines administered in a prime-boost eight-week interval regimen. Mice (n=4) were vaccinated i.m. with 1×10⁸ ifu ChAd63-[antigen] followed eight weeks later by MVA-[antigen]. Spleens were harvested at two weeks after each vaccination to assess T cell immunogenicity by ex vivo spleen IFNγ ELISpot to a pool of overlapping peptides from the appropriate antigen. Vaccines were tested in two strains of mice: (A) Balb/c, where the MVA dose was 1×10⁷ pfu and (B) C57BL/6, where the MVA dose was 1×10⁶ pfu. Results are expressed as the median SFU per million splenocytes; error bars indicate the interquartile range. Analysis of statistical difference was performed using a two-way ANOVA and a Bonferroni post-test, ****p<0.0001. For the antigen PfUIS3 two weeks post-MVA boost in Balb/c mice, the number of spots seen were at a maximum level counted by the ELISpot reader, therefore an arbitrary value of 1200 SFC per million splenocytes was assigned. Antigens are listed on the x-axis in increasing size order.

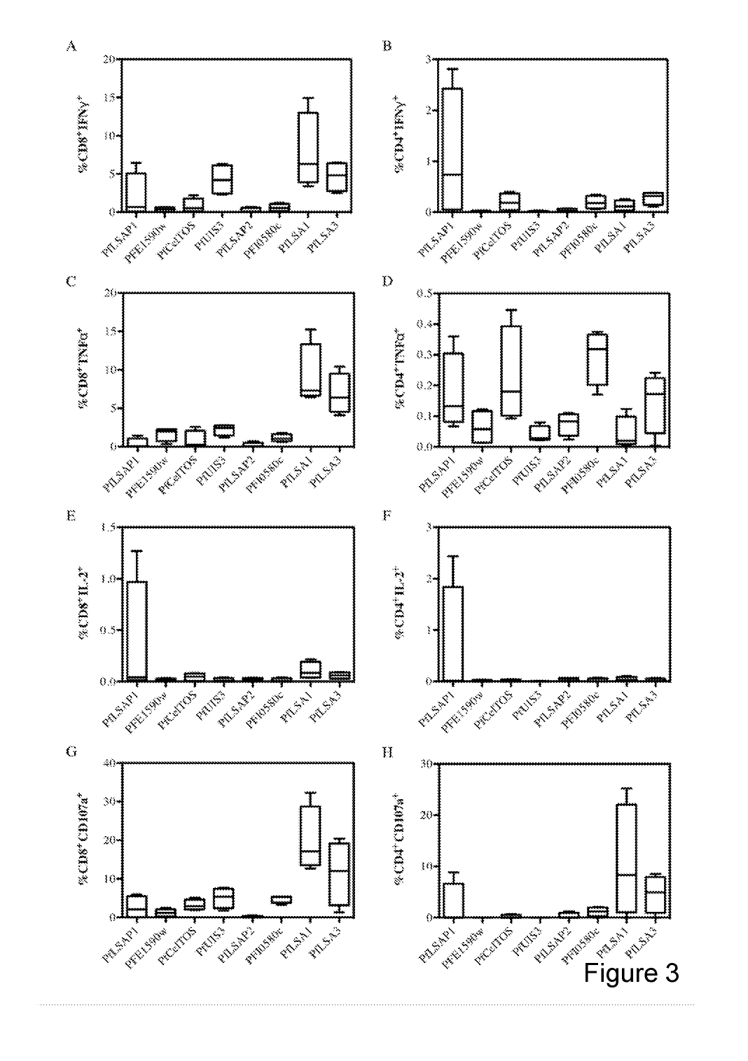

FIG. 3: CD8⁺ and CD4⁺ cytokine responses in Balb/c mice in the blood following prime-boost vaccination with the P. falciparum candidate liver-stage antigens. Balb/c mice (n=4) were vaccinated i.m. with 1×10⁸ ifu ChAd63-[antigen] followed eight weeks later by 1×10⁷ pfu MVA-[antigen]. Blood was taken one week after the final vaccination to assess CD8⁺ and CD4⁺ cytokine responses by ICS, after stimulation for six hours with a pool of overlapping peptides from the appropriate antigen. Results are expressed as the percentage of CD8⁺ (left hand side panel) or CD4⁺ (right hand side panel) T cells expressing the cytokines, with box plots indicating the median response and the whiskers showing the minimum and maximum responses. Antigens are listed on the x-axis in increasing size order. Four different markers were assessed: (A+B) IFNγ, (C+D) TNFα, (E+F) IL-2 and (G+H) the degranulation marker CD107a.

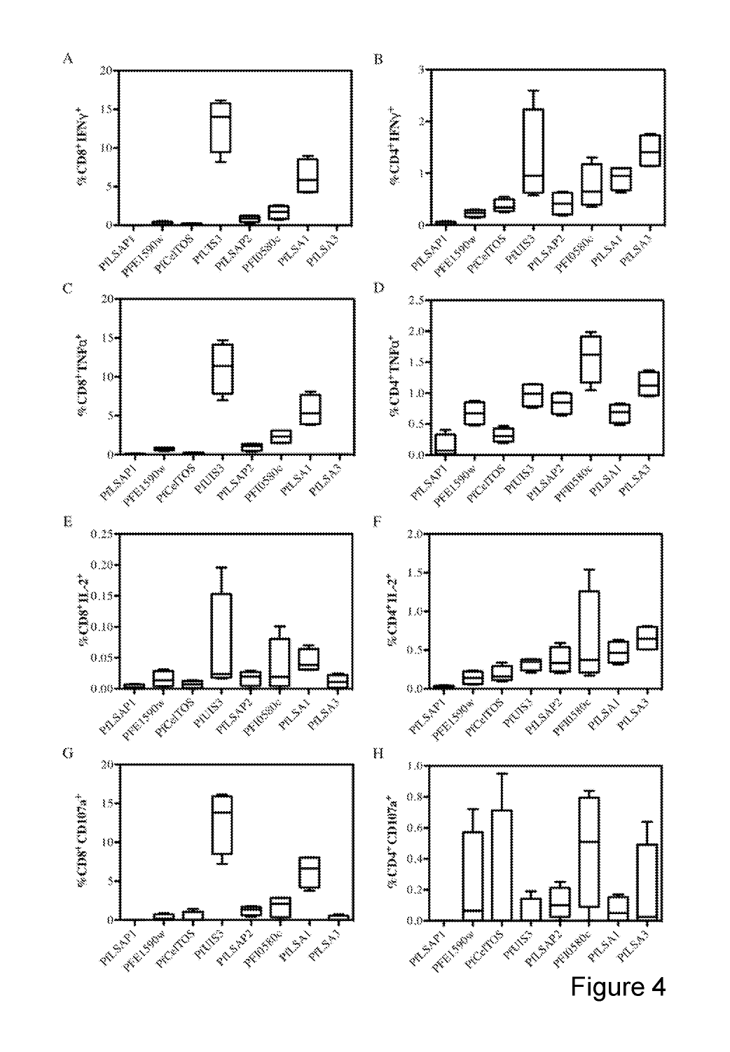

FIG. 4: CD8⁺ and CD4⁺ cytokine responses in Balb/c mice in the spleen following prime-boost vaccination with the P. falciparum candidate liver-stage antigens. Balb/c mice (n=4) were vaccinated i.m. with 1×10⁸ ifu ChAd63-[antigen] followed eight weeks later by 1×10⁷ pfu MVA-[antigen]. Spleens were harvested two weeks after the final vaccination to assess CD8⁺ and CD4⁺ cytokine responses by ICS, after stimulation for six hours with a pool of overlapping peptides from the appropriate antigen. Results are expressed as the percentage of CD8⁺ (left hand side panel) or CD4⁺ (right hand side panel) T cells expressing the cytokines, with box plots indicating the median response and the whiskers showing the minimum and maximum responses. Antigens are listed on the x-axis in increasing size order. Four different markers were assessed: (A+B) IFNγ, (C+D) TNFα, (E+F) IL-2 and (G+H) the degranulation marker CD107a.

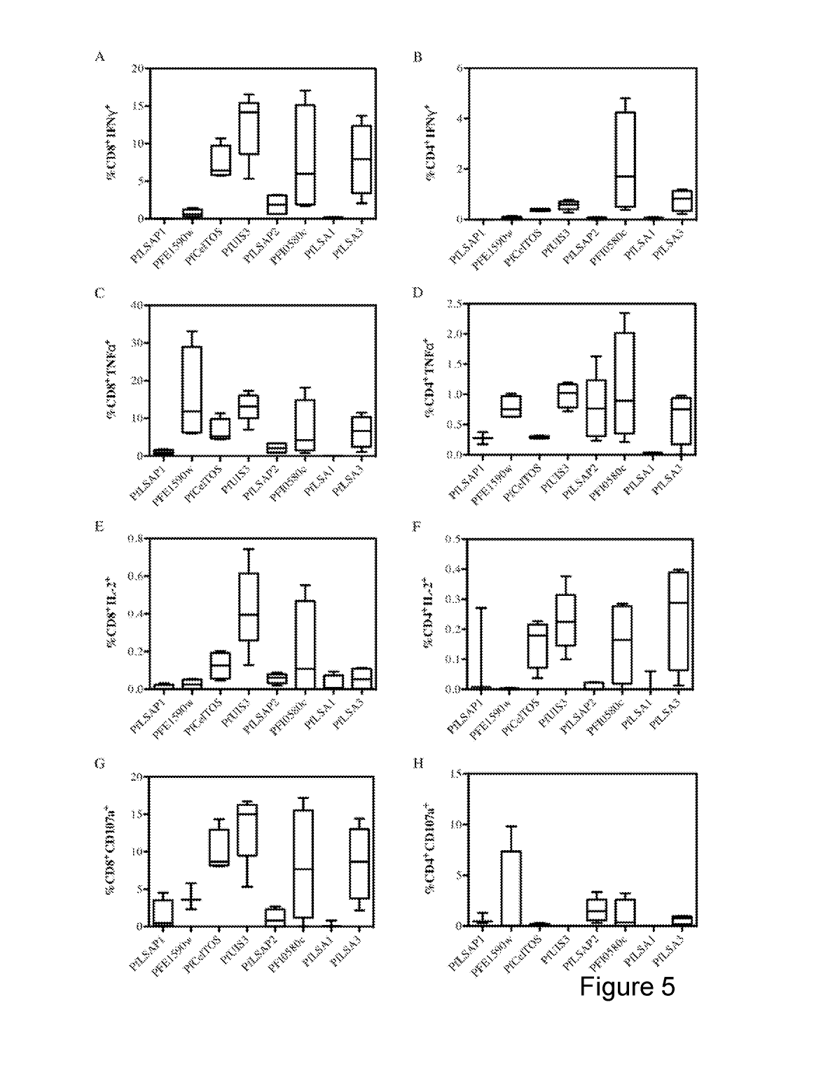

FIG. 5: CD8⁺ and CD4⁺ cytokine responses in C57BL/6 mice in the spleen following prime-boost vaccination with the P. falciparum candidate liver-stage antigens. C57BL/6 mice (n=4) were vaccinated i.m. with 1×10⁸ ifu ChAd63-[antigen] followed eight weeks later by 1×10⁶ pfu MVA-[antigen]. Spleens were harvested two weeks after the final vaccination to assess CD8⁺ and CD4⁺ cytokine responses by ICS, after stimulation for six hours with a pool of overlapping peptides from the appropriate antigen. Results are expressed as the percentage of CD8⁺ (left hand side panel) or CD4⁺ (right hand side panel) T cells expressing the cytokines, with box plots indicating the median response and the whiskers showing the minimum and maximum responses. Antigens are listed on the x-axis in increasing size order. Four different markers were assessed: (A+B) IFNγ, (C+D) TNFα, (E+F) IL-2 and (G+H) the degranulation marker CD107a.

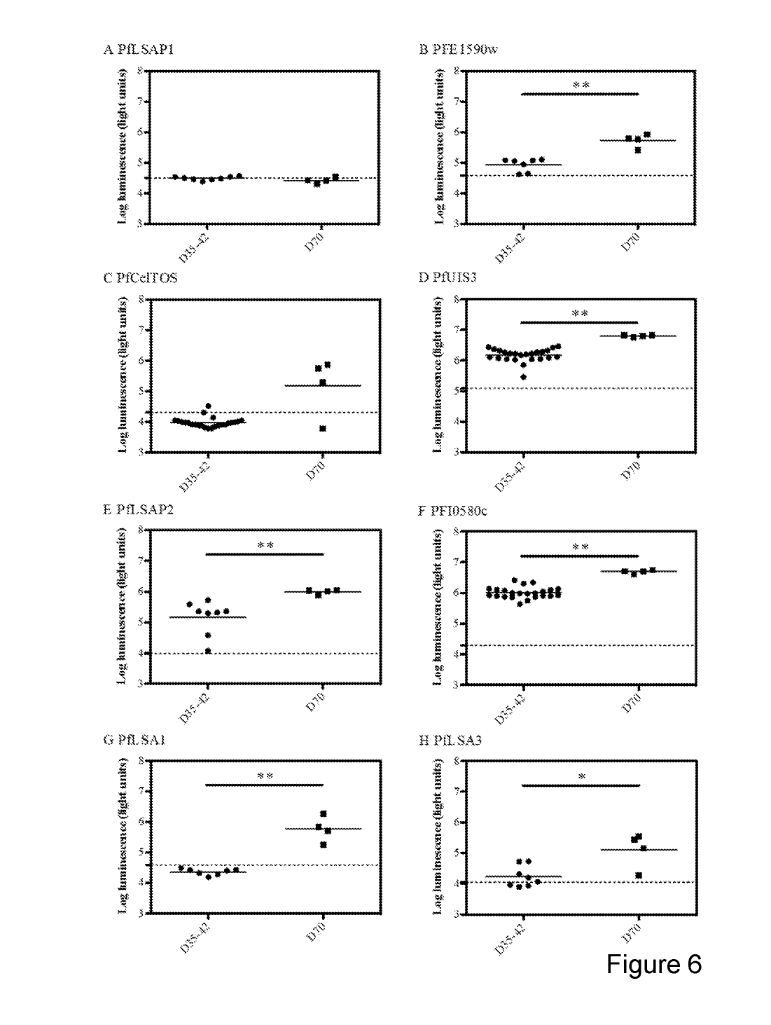

FIG. 6: Assessment of antibody responses in Balb/c mice following heterologous prime-boost vaccination with eight pre-erythrocytic candidate antigens. In the experiment described in 2.2.1 and further experiments using the same vaccination regimen, sera was collected at five to six weeks post-prime (D35-42) and two weeks post-boost (D70) and antibody levels measured by LIPS assay (n=4-24 Balb/c mice). The background response to each antigen is indicated by the dotted line, and is equal to the average of six naïve replicates plus two times the standard deviation. Raw data was log-transformed prior to analysis; results are expressed as the log luminescence (light units) measured. Both median and individual data points are shown. Statistical difference was assessed using the Mann Whitney test, *p=0.05-0.01**p=0.01-0.001.

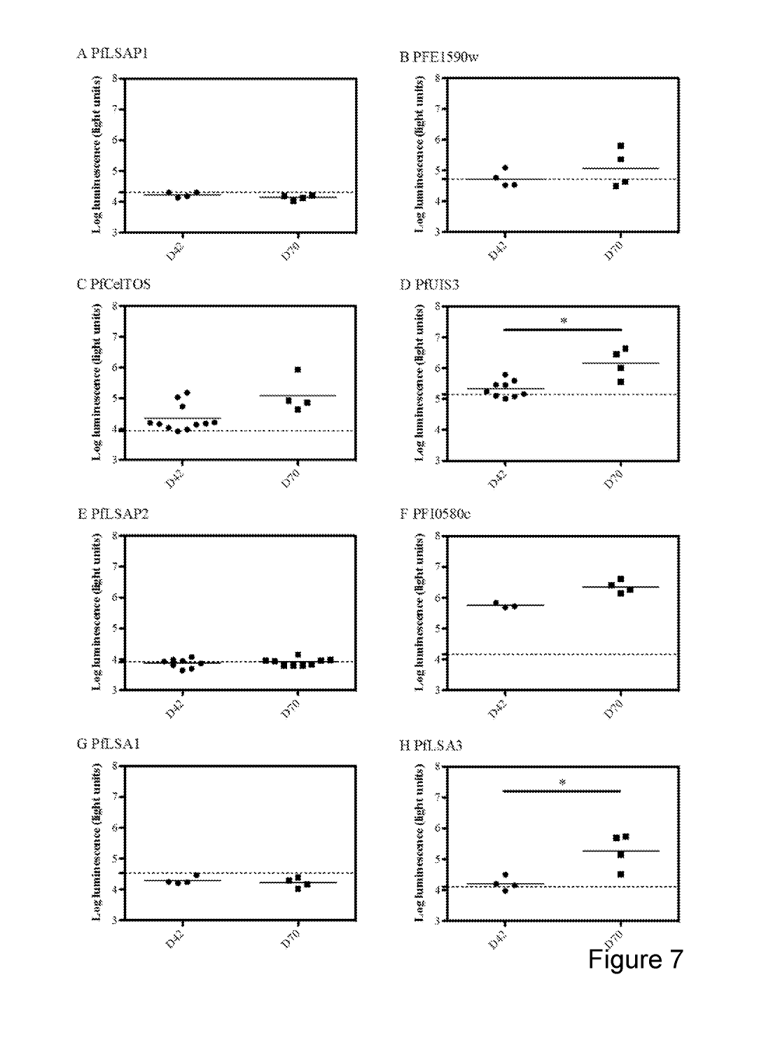

FIG. 7: Assessment of antibody responses in C57BL/6 mice following heterologous prime-boost vaccination with eight pre-erythrocytic candidate antigens. In the experiments described in 2.2.1 and further experiments using the same vaccination regimen, sera was collected at six weeks post-prime (D42) and two weeks post-boost (D70) and antibody levels measured by LIPS assay (n=3-11 C57BL/6 mice). The background response to each antigen is indicated by the dotted line, and is equal to the average of six naïve replicates plus two times the standard deviation. Raw data was log-transformed prior to analysis; results are expressed as the log luminescence (light units) measured. Both median and individual data points are shown. Statistical difference was assessed using the Mann Whitney test, *p=0.05-0.01.

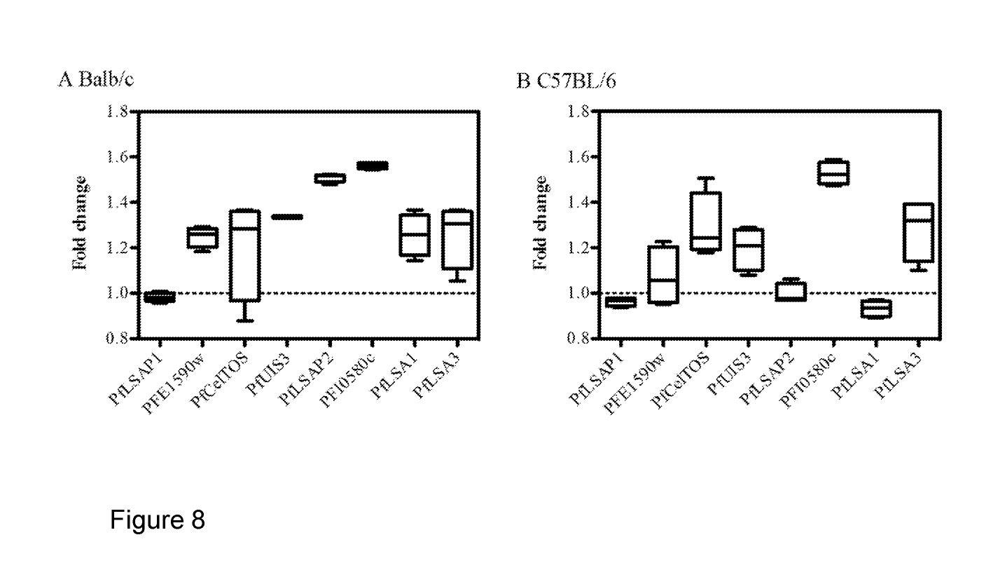

FIG. 8: Fold change in the antibody level from background to two weeks post MVA boost in (A) Balb/c and (B) C57BL/6 mice. The fold change from the background response to the antibody level post-boost was calculated for each antigen (post-boost response divided by the background response), from the data shown in FIG. 6 and FIG. 7. The background response was calculated as the average of six naïve replicates plus two times the standard deviation. Data was log transformed prior to analysis. Box plots represent the median with the whiskers indicating the maximum and minimum values. The dotted line represents no change in antibody level from the background value (=fold change of 1).

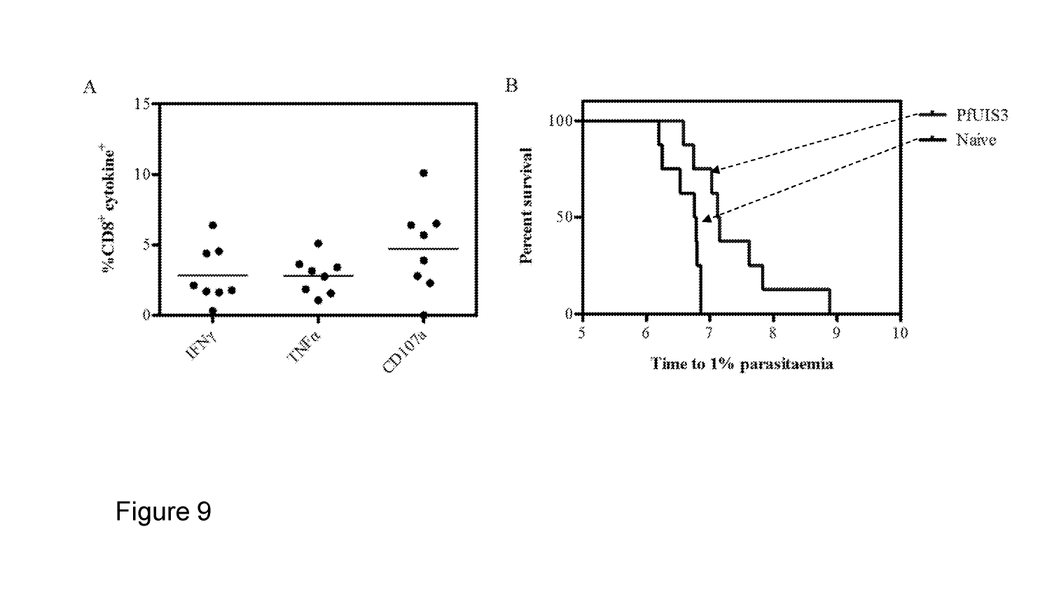

FIG. 9: Heterologous challenge with P. berghei sporozoites in Balb/c mice vaccinated with ChAd63-MVA PfUIS3. (A) Balb/c mice (n=8) were vaccinated i.m. with 1×10⁸ ifu ChAd63-PfUIS3 followed eight weeks later by 1×10⁷ pfu MVA-PfUIS3. Blood was collected six days post MVA boost to assess cellular immunogenicity by ICS, after stimulation for six hours with a pool of overlapping peptides covering the entire PfUIS3 sequence. Results are expressed as the percentage of CD8⁺ T cells expressing the cytokines IFNγ, TNFα or the degranulation marker CD107a. Both median and individual data points are shown. (B) The same mice were subsequently challenged i.v. with 1000 P. berghei sporozoites two days later (eight days post MVA boost), along with eight naïve control mice. Mice were monitored daily to enable calculation of the time to 1% parasitaemia. The Log-rank (Mantel-Cox) Test was used to assess differences between the survival curves, p=0.0048.

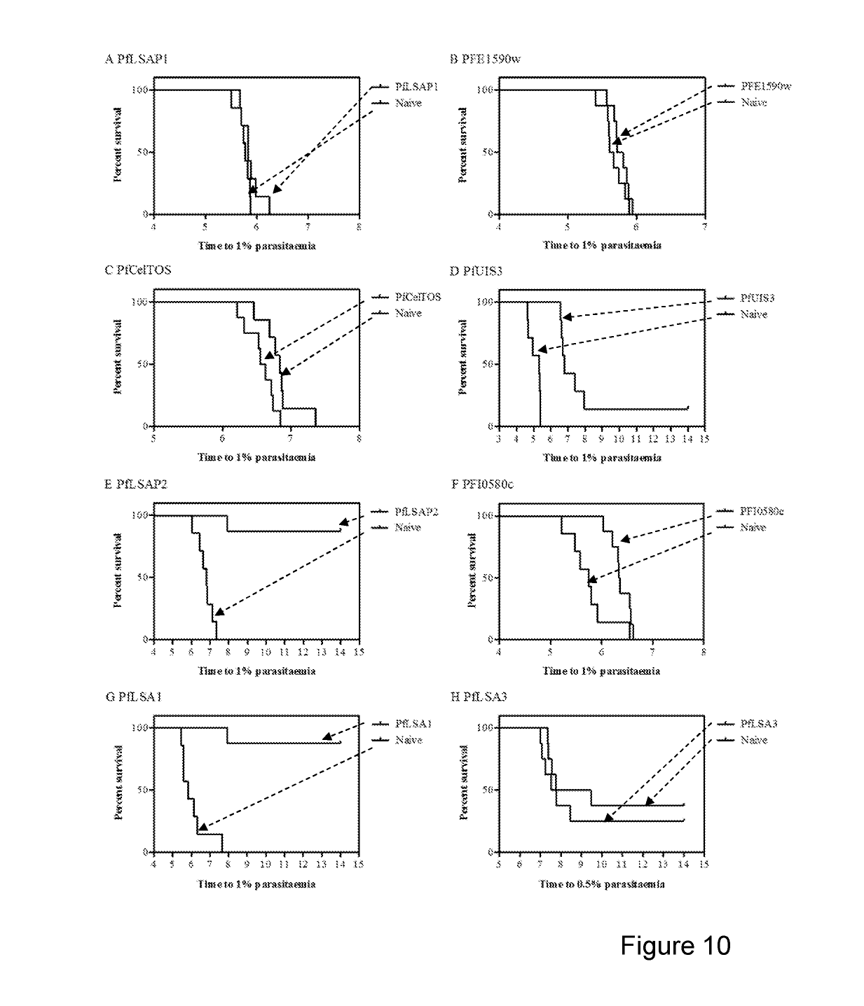

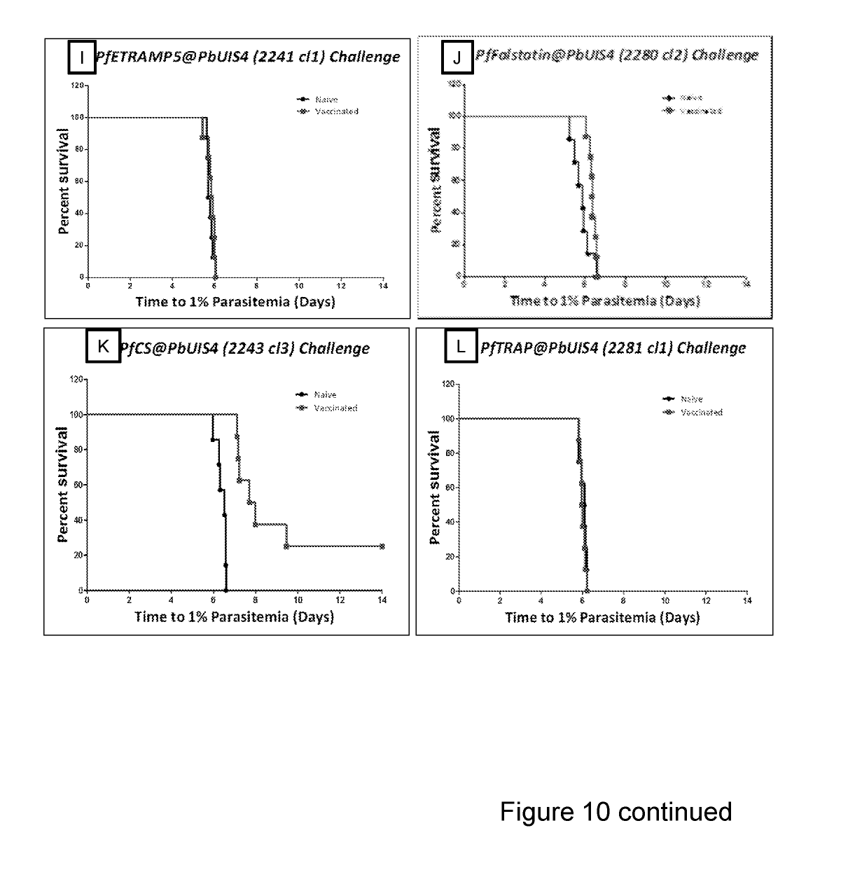

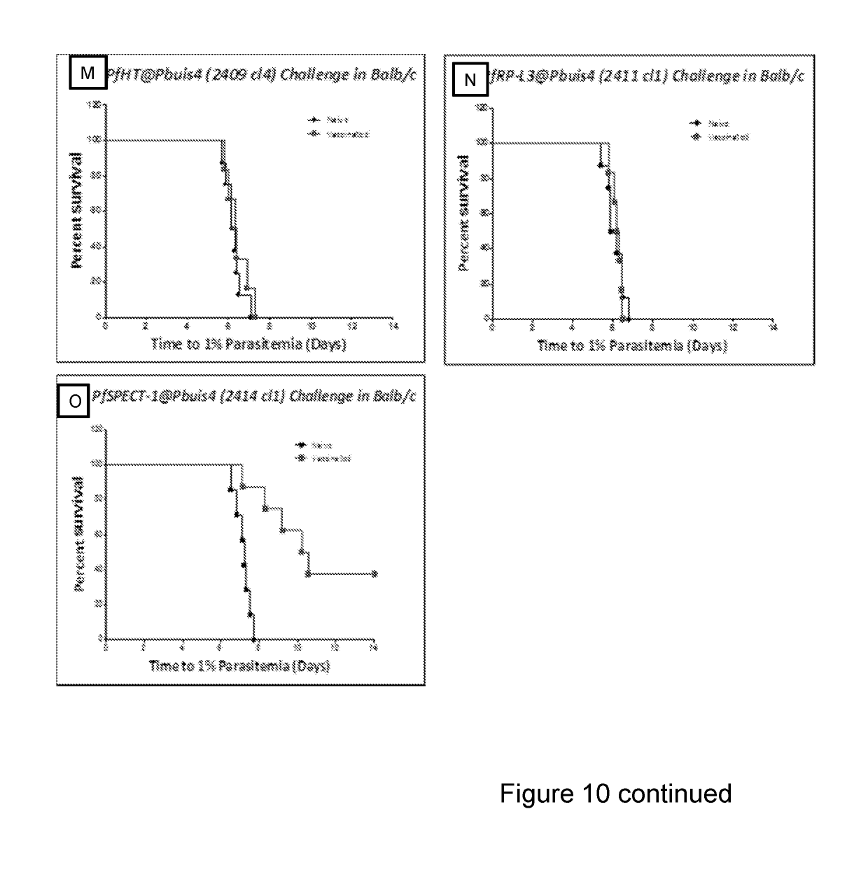

FIG. 10: Protective efficacy, as measured by time to 1% parasitaemia, after ChAd63-MVA vaccination with the P. falciparum candidate antigens and challenge with transgenic P. berghei sporozoites expressing the cognate P. falciparum antigen. Balb/c mice (n=8) were vaccinated i.m. with 1×10⁸ ifu ChAd63-[antigen] followed eight weeks later by 1×10⁷ pfu MVA-[antigen]. Blood was collected six days post MVA boost to assess cellular immunogenicity by ICS, and two days later the mice were subsequently challenged i.v. with 1000 transgenic P. berghei sporozoites expressing the cognate P. falciparum antigen. An exception was for the antigens PFI0580c, PFE1590w and PfLSAP2, where a second MVA boost was given four weeks after the first, and mice were challenged eight days after the second boost. Eight naïve mice were also challenged for each transgenic parasite line. Mice were monitored daily to enable calculation of the time to 1% parasitaemia. Mice that were slide negative at fourteen days post challenge were considered sterilely protected. The Log-rank (Mantel-Cox) Test was used to assess differences between the survival curves: (A) PfLSAP1, no significant difference (B) PFE1590w, no significant difference (C) PfCe1TOS, p=0.0291 (D) PfUIS3, p=0.0001 (E) PfLSAP2, p<0.0001 (F) PFI0580c, p=0.0072 (G) PfLSA1, p<0.0001 and (H) PfLSA3, no significant difference, (I) PfLSAP1 p=0.2, (J) PfFalstatin p=0.007, (K) PfCSP p=0.03, (L) PfTRAP p=0.3, (M) PfHT p=0.7663, (N) PfRP-L3 p=0.8562, and (O) PfSPECT-1 p=0.0023. For the PfLSA3 challenge, the chimeric sporozoite dose was increased to 2000 sporozoites per mouse in order to infect all naïve controls.

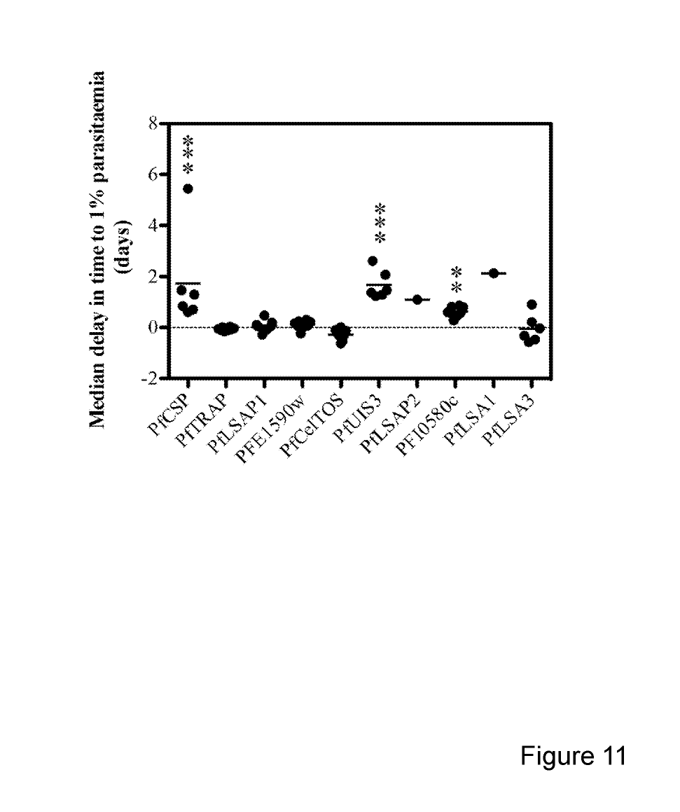

FIG. 11: Median delay in time to 1% parasitaemia following challenge with transgenic P. berghei expressing the cognate P. falciparum antigen in mice vaccinated with ChAd63-MVA Pf-[antigen]. The median delay in the time to 1% parasitaemia was calculated from the results in FIG. , using the formula: (tt1% of vaccinee)−(average tt1% of controls). All sterilely protected or non-infected mice were excluded from this analysis. Both median and individual data points are shown. Statistical significance was assessed using the Log-rank (Mantel-Cox) test on the survival curves after sterilely protected or non-infected mice were excluded, **p=0.01-0.001***p<0.001.

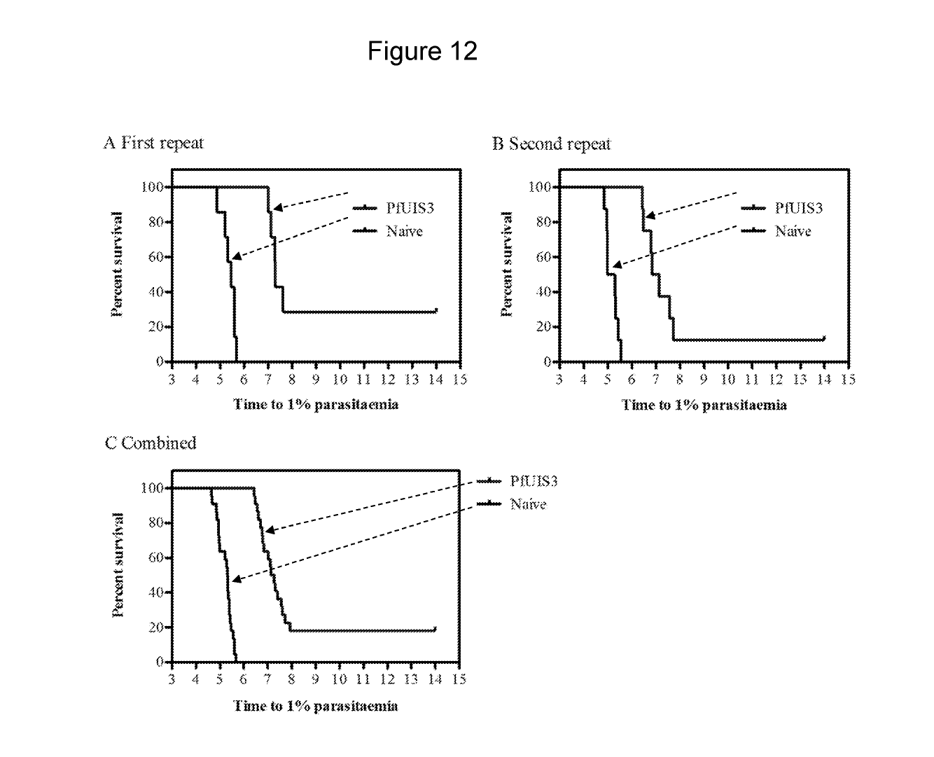

FIG. 12: Confirmation of protection in Balb/c mice induced by PfUIS3 vaccination. Balb/c mice (n=7-8 per group) were vaccinated i.m. with 1×10⁸ ifu ChAd63-PfUIS3 followed eight weeks later by 1×10⁷ pfu MVA-PfUIS3. Mice were challenged i.v. with 1000 transgenic PbPfUIS3 sporozoites ten days post-MVA boost, along with eight naïve control mice. Mice were monitored daily from four days post-challenge by thin film blood smears and the percent parasitaemia was calculated. Following three consecutive positive films, mice were culled. The data collected was used to calculate the time to 1% parasitaemia, using linear regression, and the results are presented in a survival graph. Mice that were slide-negative at fourteen days post-challenge were considered sterilely protected. The Log-rank (Mantel-Cox) Test was used to assess differences between the survival curves. Two independent experiments were conducted as shown in (A) p=0.0001 and (B), p<0.0001. (C) Results from the original and two subsequent repeat experiments were combined, p<0.0001.

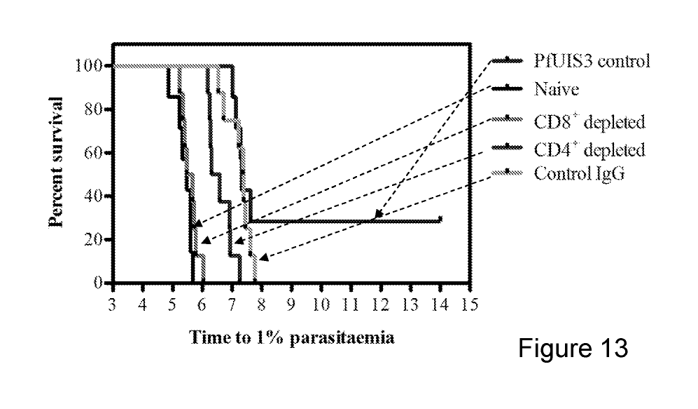

FIG. 13: Depletion of CD8⁺ T cells abolishes the protection induced by ChAd63-MVA PfUIS3 vaccination in Balb/c mice. Mice (n=4 groups of 8) were vaccinated i.m. with 1×10⁸ ifu ChAd63-PfUIS3 followed eight weeks later by 1×10⁷ pfu MVA-PfUIS3. Mice were bled seven days post-MVA boost and cellular immunogenicity assessed by intracellular cytokine staining (ICS), after stimulation for six hours with a pool of overlapping peptides to PfUIS3. No significant difference was found for any cytokine between the four groups. Mice were then injected i.p. with 100 μg of mAb to either CD4⁺ (GK1.5) or CD8⁺ (8.43) at days eight, nine and ten post-boost. One group of mice was injected with an IgG mAb control. At day ten, all mice were challenged i.v. with 1000 PbPfUIS3 sporozoites, including seven naïve controls. Mice were monitored daily to enable calculation of the time to 1% parasitaemia. Mice that were slide-negative at fourteen days post-challenge were considered sterilely protected. The Log-rank (Mantel-Cox) Test was used to assess differences between the survival curves; CD8⁻ depleted versus PfUIS3 control vaccinated p=0.0001, CD4⁻ depleted versus naïve p<0.0001, CD4⁺ depleted versus PfUIS3 control vaccinated p=0.0007.

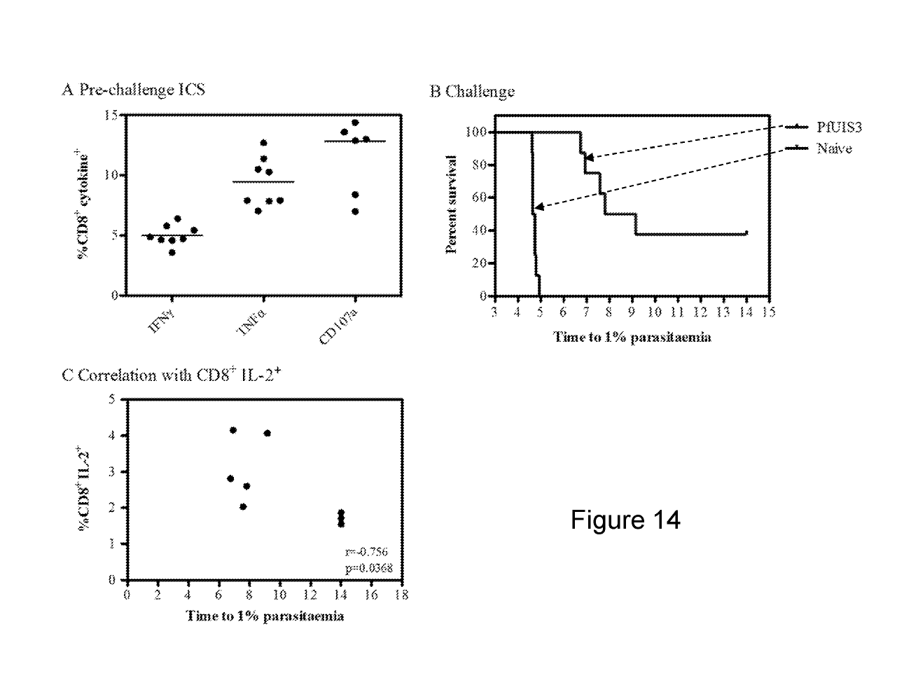

FIG. 14: ChAd63-MVA PfUIS3 vaccination induces protection against sporozoite challenge in C57BL/6 mice. C57BL/6 mice (n=8) were vaccinated i.m. with 1×10⁸ ifu ChAd63-PfUIS3 followed eight weeks later by 1×10⁶ pfu MVA-PfUIS3. Blood was taken seven days post-boost to assess both humoral and cellular immunogenicity. (A) Cellular immunogenicity was assessed by ICS, after stimulation for six hours with an overlapping peptide pool to PfUIS3. Both median and individual data points are shown. (B) Mice were challenged i.v. with 1000 transgenic PbPfUIS3 sporozoites ten days post-MVA boost, along with eight naïve control mice. Mice were monitored daily to enable calculation of the time to 1% parasitaemia. Mice that were slide-negative at fourteen days post-challenge were considered sterilely protected. The Log-rank (Mantel-Cox) Test was used to assess differences between the survival curves, p<0.0001. (C) Correlations were assessed between the time to 1% parasitaemia and both cellular and humoral immunogenicity. The only correlation identified was with CD8⁺ IL-2⁺ cells, Spearman r=−0.756 p=0.0368.

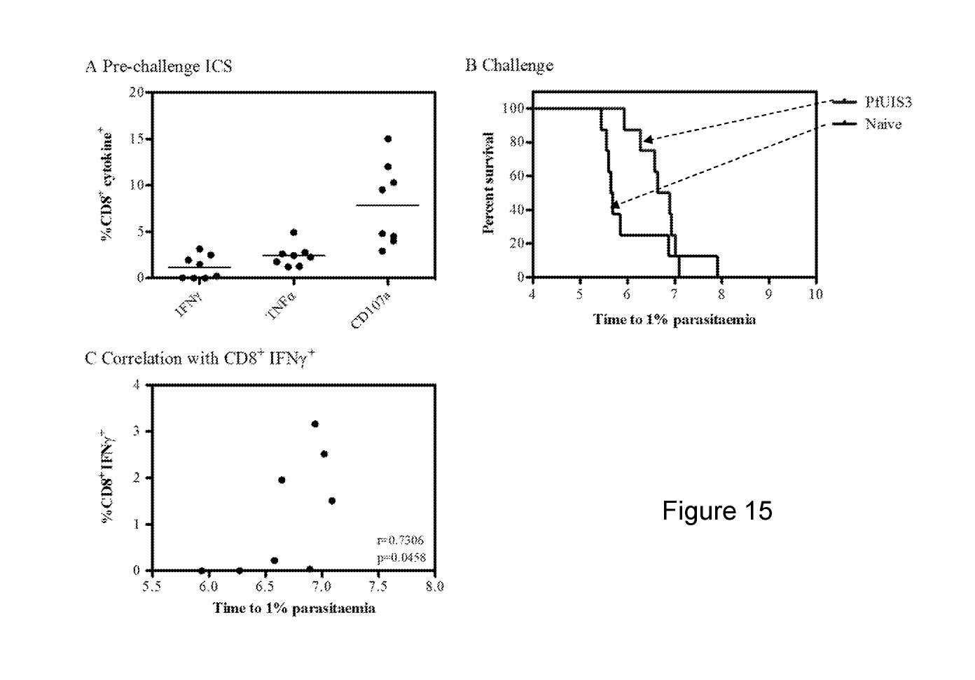

FIG. 15: ChAd63-MVA PfUIS3 vaccination does not induce protection against sporozoite challenge in CD-1 outbred mice. CD-1 mice (n=8) were vaccinated i.m. with 1×10⁸ ifu ChAd63-PfUIS3 followed eight weeks later by 1×10⁷ pfu MVA-PfUIS3. Blood was taken seven days post-boost to assess both humoral and cellular immunogenicity. (A) Cellular immunogenicity was assessed by ICS, after stimulation for six hours with an overlapping peptide pool to PfUIS3. Both median and individual data points are shown. (B) Mice were challenged i.v. with 1000 transgenic PbPfUIS3 sporozoites ten days post-MVA boost, along with eight naïve control mice. Mice were monitored daily to enable calculation of the time to 1% parasitaemia. The Log-rank (Mantel-Cox) Test was used to assess differences between the survival curves, no difference was found. (C) Correlations were assessed between the time to 1% parasitaemia and both cellular and humoral immunogenicity. Correlations were identified with both CD8⁺ IFNγ⁺ cells, Spearman r=−0.756 p=0.0368 as shown, and CD8⁻ TNFα⁺ cells, Spearman r=0.7857 p=0.0279.

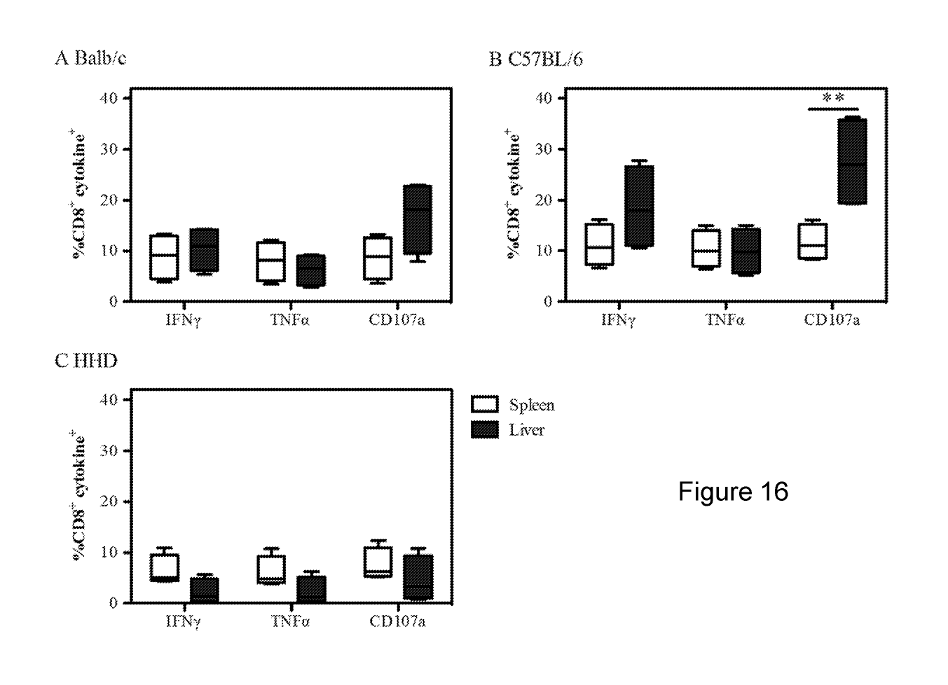

FIG. 16: PfUIS3-specific cells were observed in both the liver and spleen of mice after ChAd63-MVA vaccination. Livers were harvested from mice sacrificed two-weeks post-boost, following perfusion in situ. Single cell suspensions of liver and spleen mononuclear cells were isolated and stimulated for six hours with an overlapping peptide pool to PfUIS3. The percentage of CD8⁺ cytokine⁺ cells are shown for (A) Balb/c (B) C57BL/6 and (C) HHD mice. Box plots indicate the median response with whiskers representing the minimum and maximum responses. Statistical difference was assessed using a two-way ANOVA with Bonferroni post-test; the only difference was for C57BL/6 mice where the CD8⁺ CD107a⁺ response observed in the liver was greater than in the spleen, **p<0.01.

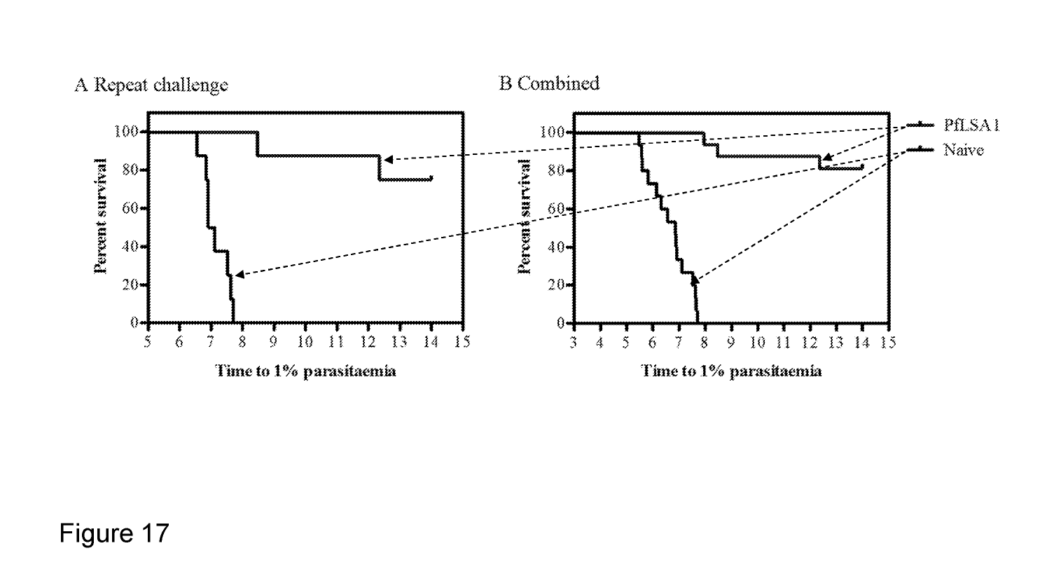

FIG. 17: Confirmation of pre-erythrocytic protection in Balb/c mice induced by PfLSA1 vaccination. (A) Balb/c mice (n=8) were vaccinated i.m. with 1×10⁸ ifu ChAd63-PfLSA1 followed eight weeks later by 1'10⁷ pfu MVA-PfLSA1. Mice were challenged i.v. with 1000 transgenic PbPfLSA1 sporozoites ten days post-MVA boost, along with eight naïve control mice. Mice were monitored daily to enable calculation of the time to 1% parasitaemia. Mice that were slide-negative at fourteen days post-challenge were considered sterilely protected. The Log-rank (Mantel-Cox) Test was used to assess differences between the survival curves, p<0.0001. (B) Results from the repeat and the original experiment were combined (vaccinated n=16, naïve n=15), p<0.0001.

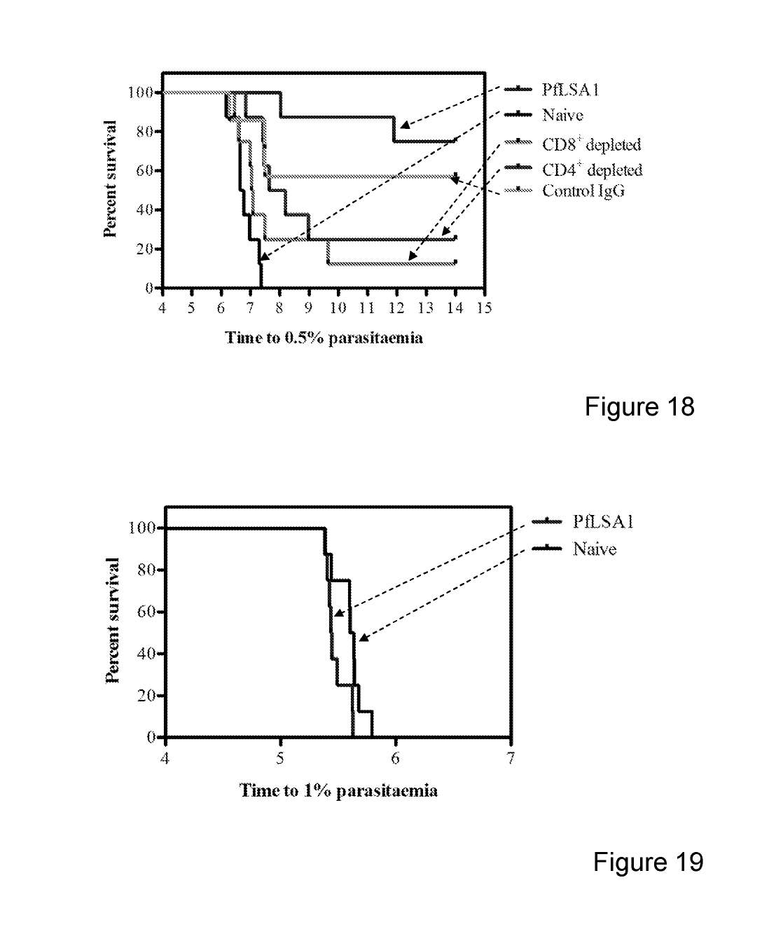

FIG. 18: Depletion of CD8⁺ T cells abolishes the protection induced by ChAd63-MVA PfLSA1 vaccination in Balb/c mice. Mice (n=4 groups of 7-8) were vaccinated i.m. with 1×10⁸ ifu ChAd63-PfLSA1 followed eight weeks later by 1×10⁷ pfu MVA-PfLSA1. Mice were bled seven days post-MVA boost and cellular immunogenicity assessed by ICS, after stimulation for six hours with a pool of overlapping peptides to PfLSA1. No significant difference was found for any cytokine between the four groups. Mice were injected i.p. with 100 μg of mAb to either CD4⁺ (GK1.5) or CD8⁺ (8.43) at days eight, nine and ten post-boost. One group of mice was injected with an IgG mAb control. At day ten, all mice were challenged i.v. with 1000 PbPfLSA1 sporozoites, including eight naïve control mice. Mice were monitored daily to enable calculation of the time to 0.5% parasitaemia. Mice that were slide-negative at fourteen days post-challenge were considered sterilely protected. The Log-rank (Mantel-Cox) Test was used to assess differences between the survival curves; CD8⁺ depleted versus PfLSA1 control vaccinated p=0.0027, CD4⁻ depleted versus naïve p=0.0003, CD4⁺ depleted versus PfLSA1 control vaccinated p=0.0027.

FIG. 19: ChAd63-MVA PfLSA1 vaccination does not induce protection against sporozoite challenge in C57BL/6 mice. C57BL/6 mice (n=8) were vaccinated i.m. with 1×10⁸ ifu ChAd63-PfLSA1 followed eight weeks later by 1×10⁷ pfu MVA-PfLSA1. Mice were challenged i.v. with 1000 transgenic PbPfLSA1 sporozoites ten days post-MVA boost, along with eight naïve control mice. Mice were monitored daily to enable calculation of the time to 1% parasitaemia. The Log-rank (Mantel-Cox) Test was used to assess differences between the survival curves, no difference was found.

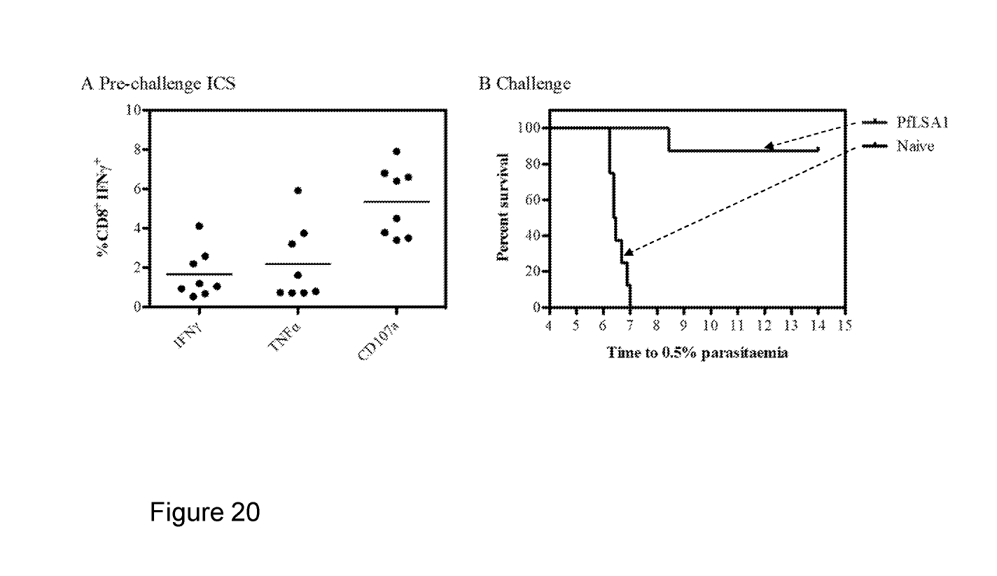

FIG. 20: ChAd63-MVA PfLSA1 vaccination induces protection against sporozoite challenge in CD-1 outbred mice. CD-1 mice (n=8) were vaccinated i.m. with 1×10⁸ ifu ChAd63-PfLSA1 followed eight weeks later by 1×10⁷ pfu MVA-PfLSA1. Blood was taken seven days post-boost to assess both humoral and cellular immunogenicity. (A) Cellular immunogenicity was assessed by ICS, after stimulation for six hours with an overlapping peptide pool to PfLSA1. Both median and individual data points are shown. (B) Mice were challenged i.v. with 1000 transgenic PbPfLSA1 sporozoites ten days post-MVA boost, along with eight naïve control mice. Mice were monitored daily to enable calculation of the time to 0.5% parasitaemia. Mice that were slide-negative at fourteen days post-challenge were considered sterilely protected. The Log-rank (Mantel-Cox) Test was used to assess differences between the survival curves, p<0.0001.

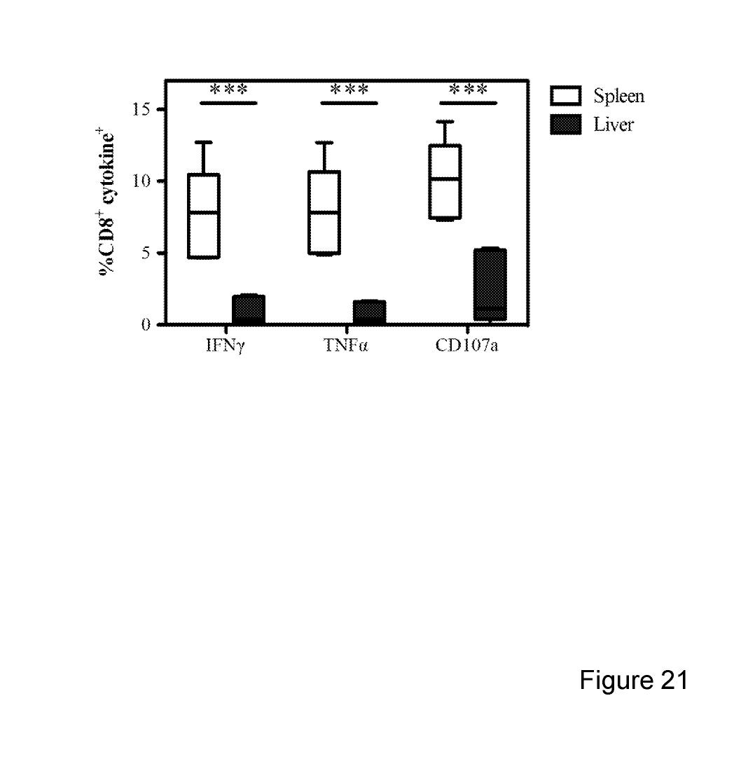

FIG. 21: ChAd63-MVA PfLSA1 vaccination in Balb/c mice induces a low magnitude antigen-specific cellular response in the liver. Livers were harvested from mice sacrificed two-weeks post-boost, following perfusion in situ. Single cell suspensions of spleen and liver mononuclear cells were stimulated for six hours with an overlapping peptide pool to PfLSA1, and the percentage of CD8⁺ cytokine⁻ cells are shown. Box plots indicate the median response with whiskers representing the minimum and maximum responses. Statistical difference between the response detected in the spleen and liver was assessed by two-way ANOVA with Bonferroni post-test, ***p<0.001, overall p<0.0001.

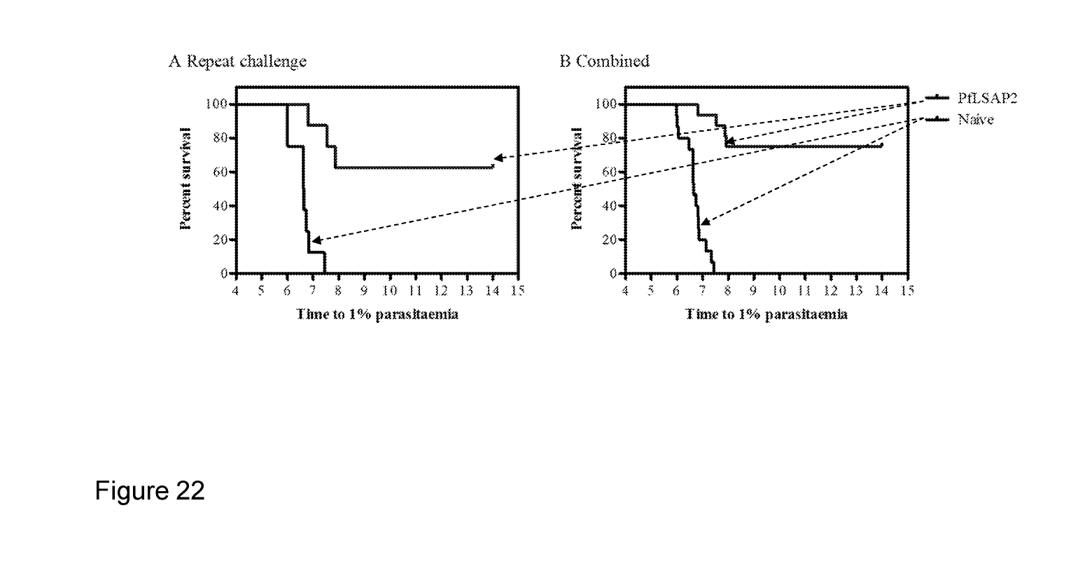

FIG. 22: Confirmation of protection in Balb/c mice induced by PfLSAP2 vaccination. (A) Balb/c mice (n=8) were vaccinated i.m. with 1×10⁸ ifu ChAd63-PfLSAP2 followed eight weeks later by 1×10⁷ pfu MVA-PfLSAP2. Mice were challenged i.v. with 1000 transgenic PbPfLSAP2 sporozoites ten days post-MVA boost, along with eight naïve control mice. Mice were monitored daily to enable calculation of the time to 1% parasitaemia. Mice that were slide-negative at fourteen days post-challenge were considered sterilely protected. The Log-rank (Mantel-Cox) Test was used to assess differences between the survival curves, p=0.0002. (B) Results from the repeat and the original experiment were combined (vaccinated n=16, naïve n=15), p<0.0001.

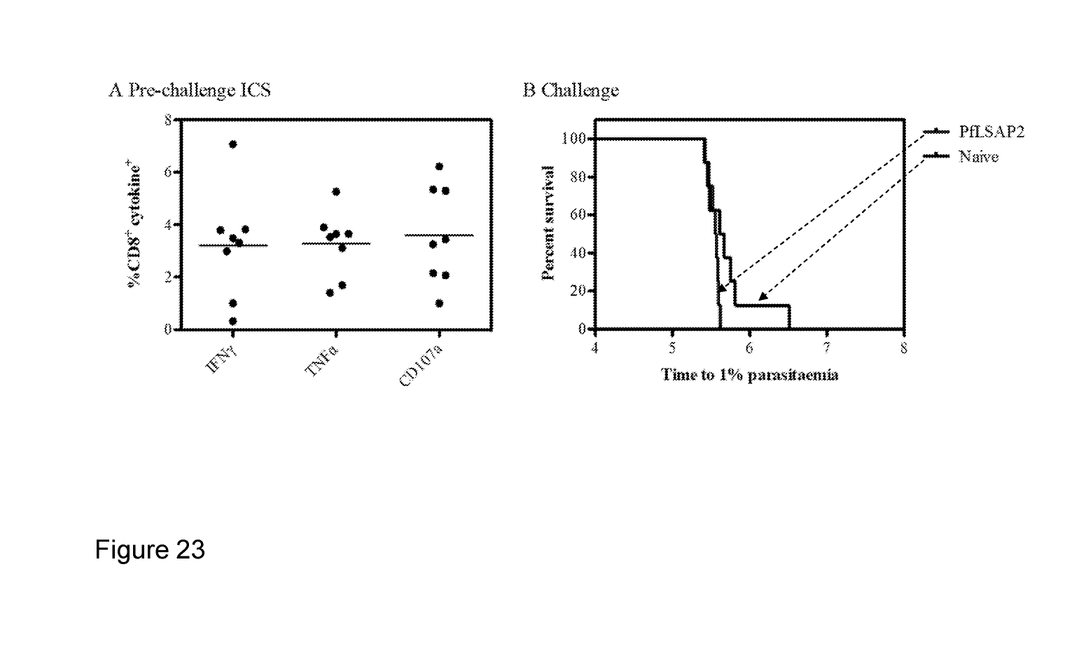

FIG. 23: ChAd63-MVA PfLSAP2 vaccination does not induce protection against sporozoite challenge in C57BL/6 mice. C57BL/6 mice (n=8) were vaccinated i.m. with 1×10⁸ ifu ChAd63-PfLSAP2 followed eight weeks later by 1×10⁷ pfu MVA-PfLSAP2. Blood was taken seven days post-boost to assess both humoral and cellular immunogenicity. (A) Cellular immunogenicity was assessed by ICS, after stimulation for six hours with an overlapping peptide pool to PfLSAP2. Both median and individual data points are shown. (B) Mice were challenged i.v. with 1000 transgenic PbPfLSAP2 sporozoites ten days post-MVA boost, along with eight naïve control mice. Mice were monitored daily to enable calculation of the time to 1% parasitaemia was calculated. The Log-rank (Mantel-Cox) Test was used to assess differences between the survival curves, no difference was found.

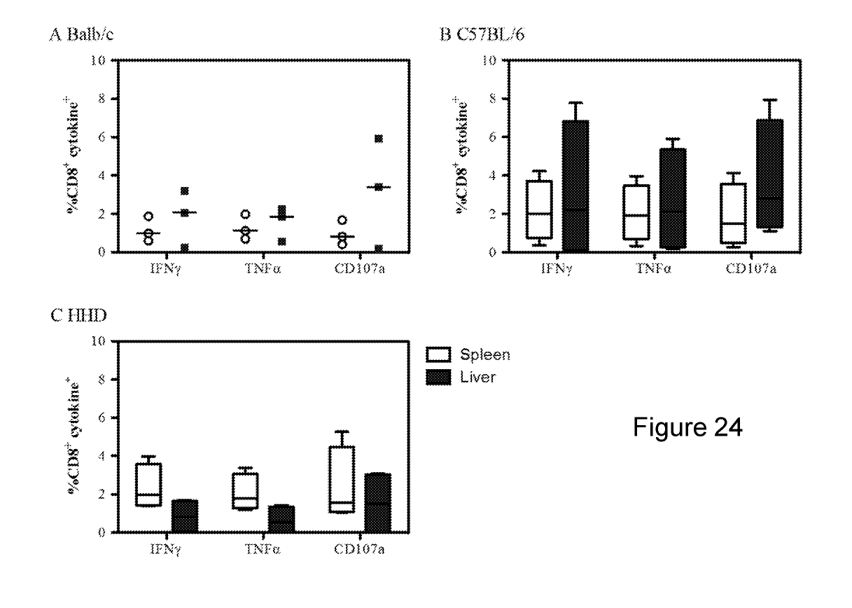

FIG. 24: ChAd63-MVA PfLSAP2 vaccination induces an antigen-specific cellular response in the liver. Livers were harvested from mice sacrificed two-weeks post-boost, following perfusion in situ. Single cell suspensions of spleen and liver mononuclear cells were isolated and stimulated for six hours with an overlapping peptide pool to PfLSAP2. The percentage of CD8⁺ cytokine⁺ cells are shown for (A) Balb/c (B) C57BL/6 and (C) HHD mice. Box plots indicate the median response with whiskers representing the minimum and maximum responses. As only three mice were assayed for Balb/c, individual data points are shown. Statistical difference between the spleen and liver responses was assessed using a two-way ANOVA with Bonferroni post-test, no differences were observed.

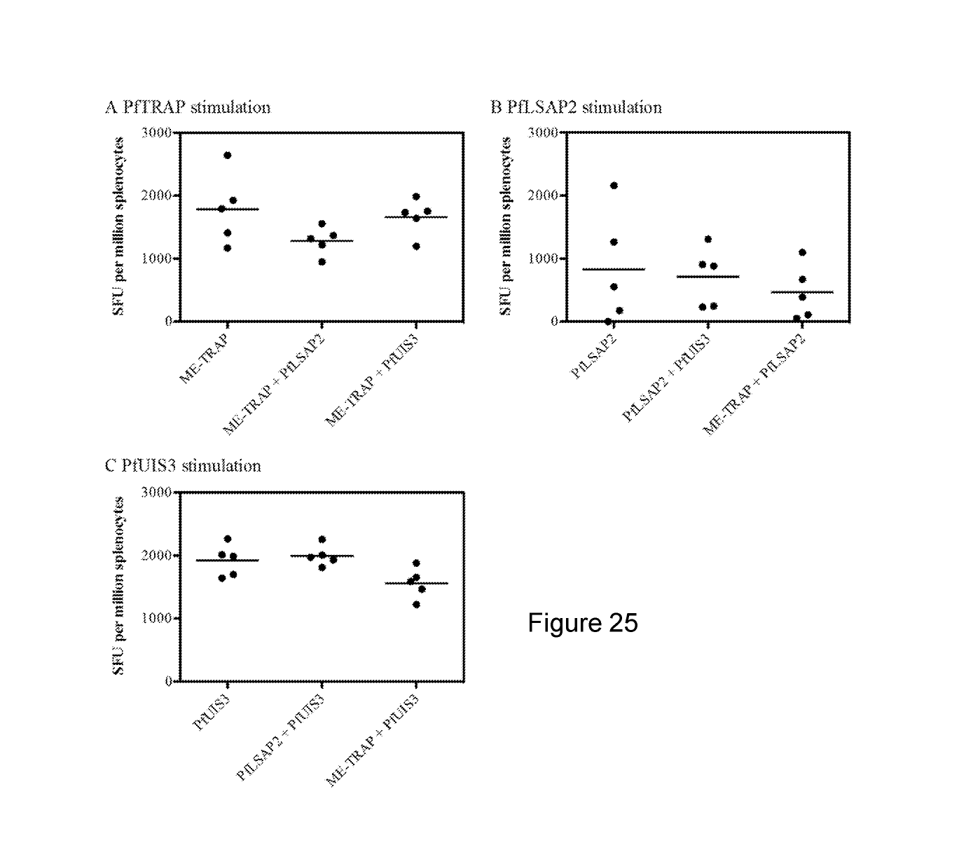

FIG. 25: Vaccination with combinations of PfUIS3 and PfLSAP2 with ME-TRAP, or with each other, does not result in reduced cellular immunogenicity in C57BL/6 mice compared to each vaccine given alone. C57BL/6 mice (n=5 per group) were vaccinated i.m. with 1×10⁸ ifu ChAd63 followed eight weeks later by 1×10⁷ pfu MVA, with antigens indicated on the x-axis. When two vaccines were given, mice were vaccinated with a full dose of each vaccine administered in separate legs. Two weeks post-MVA boost, mice were sacrificed and splenocytes were isolated to perform an ex vivo IFNγ ELISpot. Splenocytes were stimulated with an overlapping peptide pool to (A) PfTRAP (T9/96), (B) PfLSAP2 or (C) PfUIS3. Both median and individual data points are shown. The Kruskal-Wallis Test with Dunn's Multiple Comparison Test was used to assess statistical difference between groups. No differences were found.

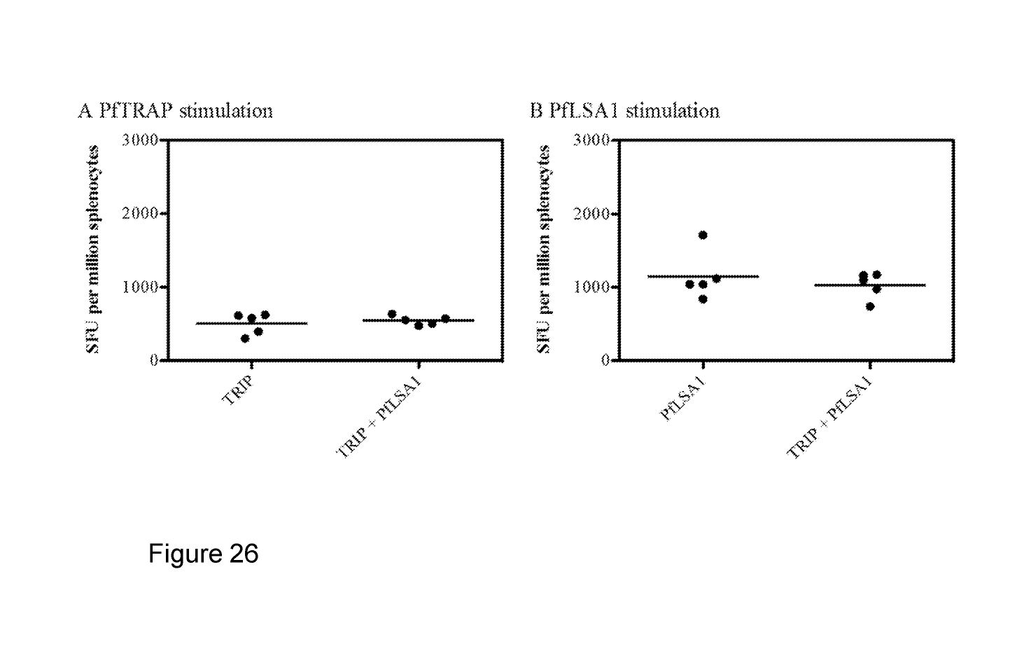

FIG. 26: Vaccination with both PfLSA1 and TRIP does not result in reduced cellular immunogenicity in Balb/c mice compared to vaccination with either alone. Balb/c mice (n=5 per group) were vaccinated i.m. with 1×10⁸ ifu ChAd63 followed eight weeks later by 1×10⁷ pfu MVA, with antigens indicated on the x-axis. When two vaccines were given, mice were vaccinated with a full dose of each vaccine administered in separate legs. Two weeks post-MVA boost, mice were sacrificed and splenocytes were isolated to perform an ex vivo IFNγ ELISpot. Splenocytes were stimulated with an overlapping peptide pool to (A) PfTRAP (3D7) or (B) PfLSA1. Both median and individual data points are shown. The Mann Whitney test was used to assess statistical difference between groups. No differences were found.

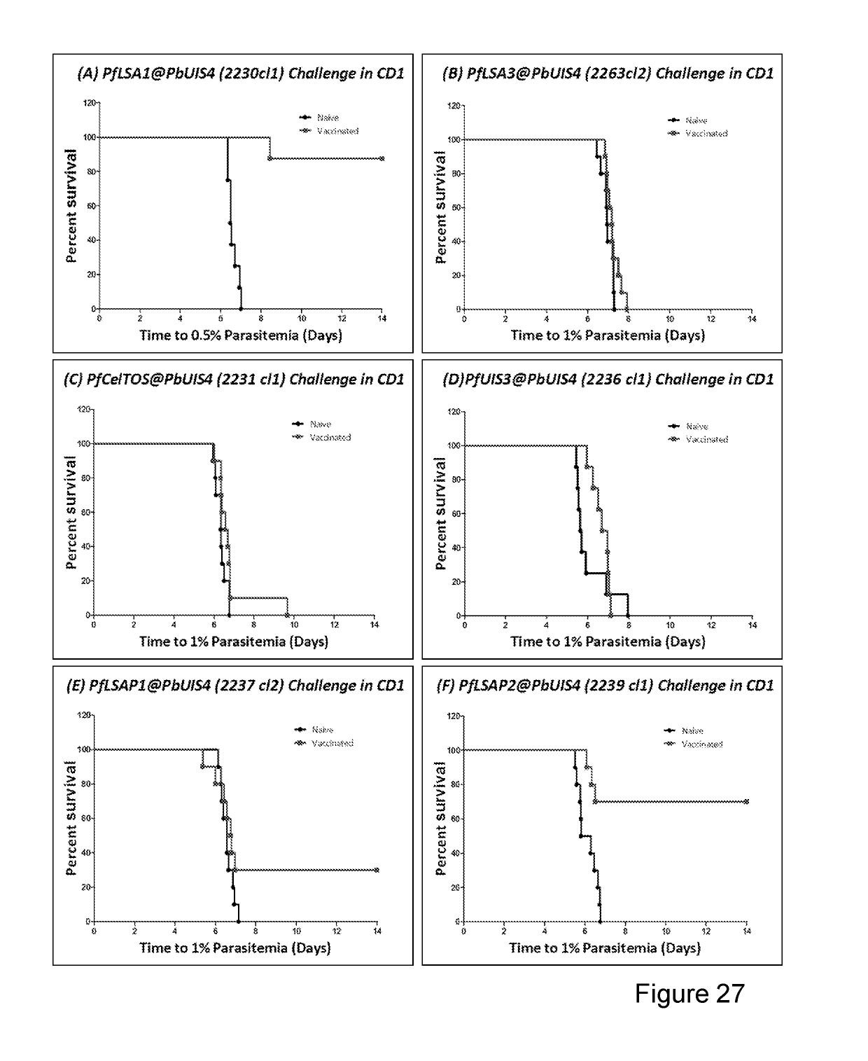

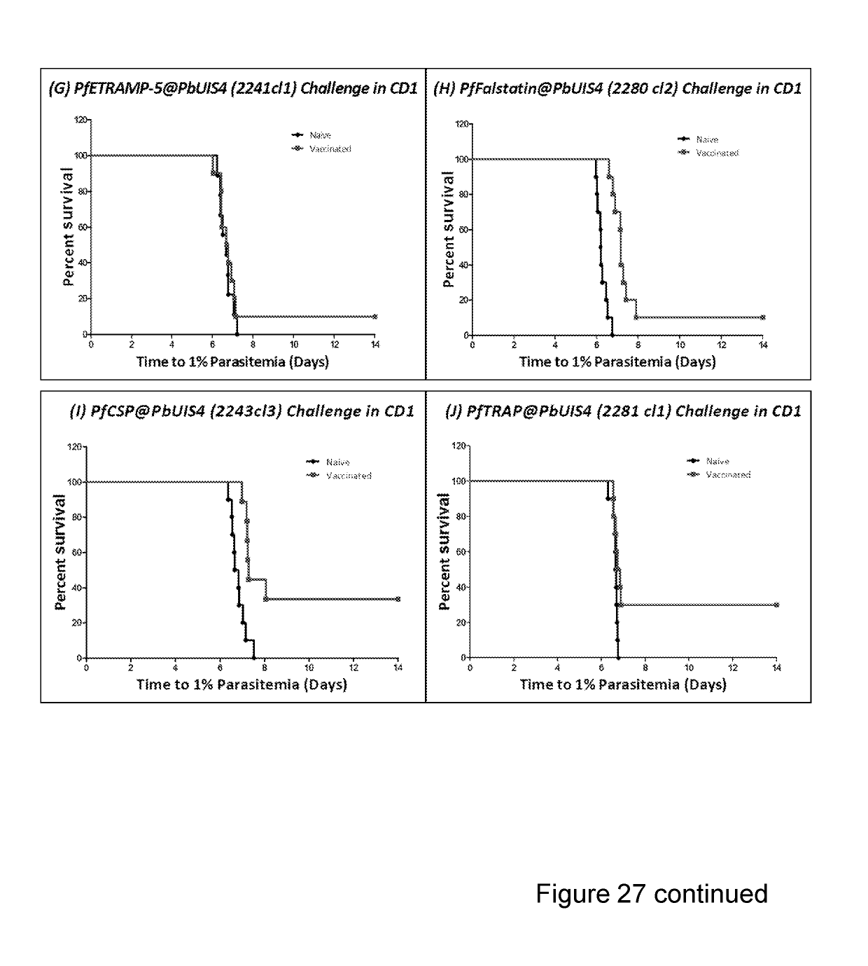

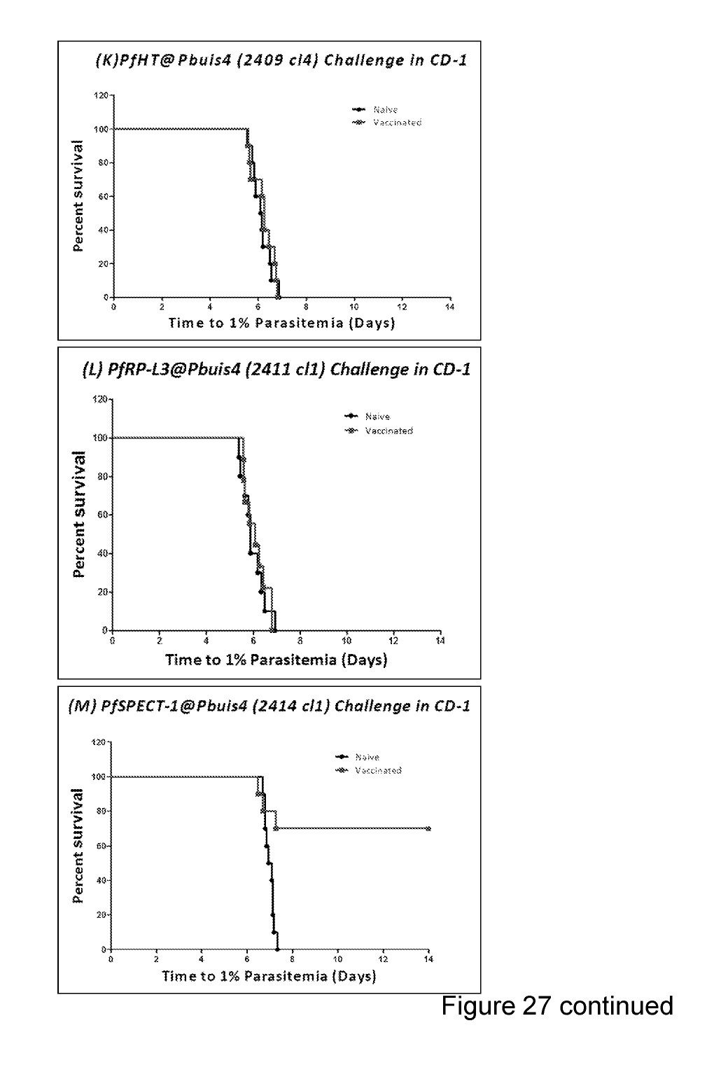

FIG. 27: Protective efficacy of the ChAd63-MVA P. falciparum vaccines in CD-1 outbred mice (n=8-10 vaccinated and 8-10 naive). CD-1 mice were challenged with 1000 chimeric sporozoites i.v. The Kaplan-Meier curves illustrate the time to 0.5% or 1% parasitaemia, whilst statistical significance between the survival curves was assessed using the Log-Rank (Mantel-Cox) Test. (A) PfLSA1 p<0.0001, (B) PfLSA3 p=0.1506, (C) PfCe1TOS p=0.0971, (D) PfUIS3 p=0.2518, (E) PfLSAP1 p=0.1564, (F) PfLSAP2 p=0.0.0009, (G) PfETRAMP5 p=0.4548, (H) PfFalstatin p<0.0001, (I) PfCSP p=0.0011, (J) PfTRAP p=0.0227, (K) PfHT p=0.7663. (L) PfRP-L3 p=0.8562. (M) PfSPECT-1 p=0.0023. For the PfLSA3 challenge, the chimeric sporozoite dose was increased to 2000 sporozoites per mouse in order to infect all naïve controls.

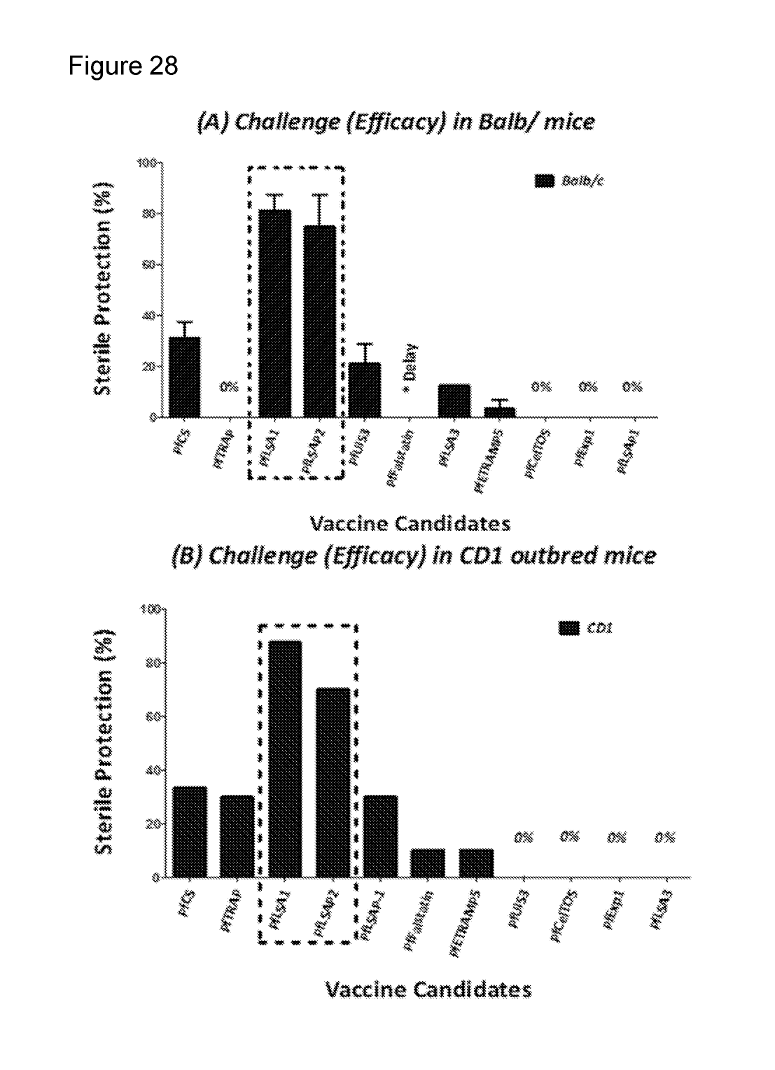

FIG. 28: The protective efficacy Rank/order of the eight novel P. falciparum viral vaccine candidates. Efficacy is compared to the current two leading malaria vaccines PfCSP and PfTRAP using the transgenic parasite challenging model. Strong protective immunity against PfLSA1 and PfLSAP2 in both (A) inbred Balb/c, and (B) outbred CD1 mice.

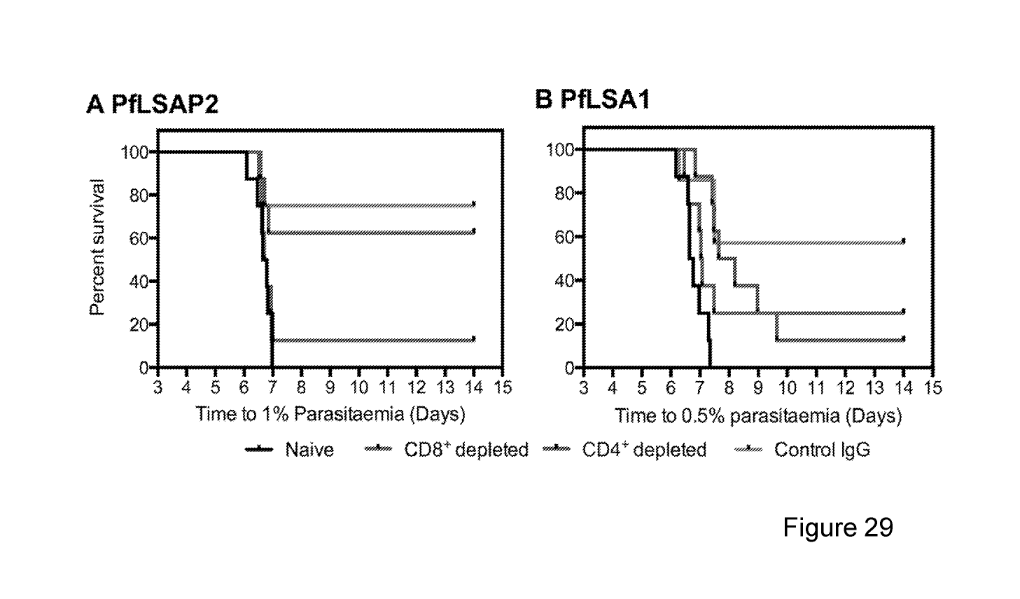

FIG. 29: CD8+ T cells are required for protective efficacy elicited by ChAd63-MVA PfLSA1 or PfLSAP2. (A and B) BALB/c mice (n=7-8 per group) were injected with the appropriate monoclonal antibody to deplete CD4+ or CD8+ T cells, or with an unrelated IgG control, and challenged with 1000 chimeric parasites i.v. ten days after ChAd63-MVA vaccination. Naive mice acted as another control. The Kaplan-Meier curves illustrate the time to 0.5 or 1% parasitaemia, and the Log-Rank (Mantel-Cox) Test was used to compare groups of mice. For PfLSAP2 (A): CD8+ depleted vs naïve, not significant (NS); CD8+ depleted vs control IgG, p=0.03; CD4+ depleted vs naïve, p=0.01; and CD4+ depleted vs control IgG, NS. For PfLSA1 (B): CD8+ depleted vs naïve, NS; CD8+ depleted vs control IgG, NS; CD4+ depleted vs naïve, p=0.0003; CD4+depleted vs control IgG, NS.

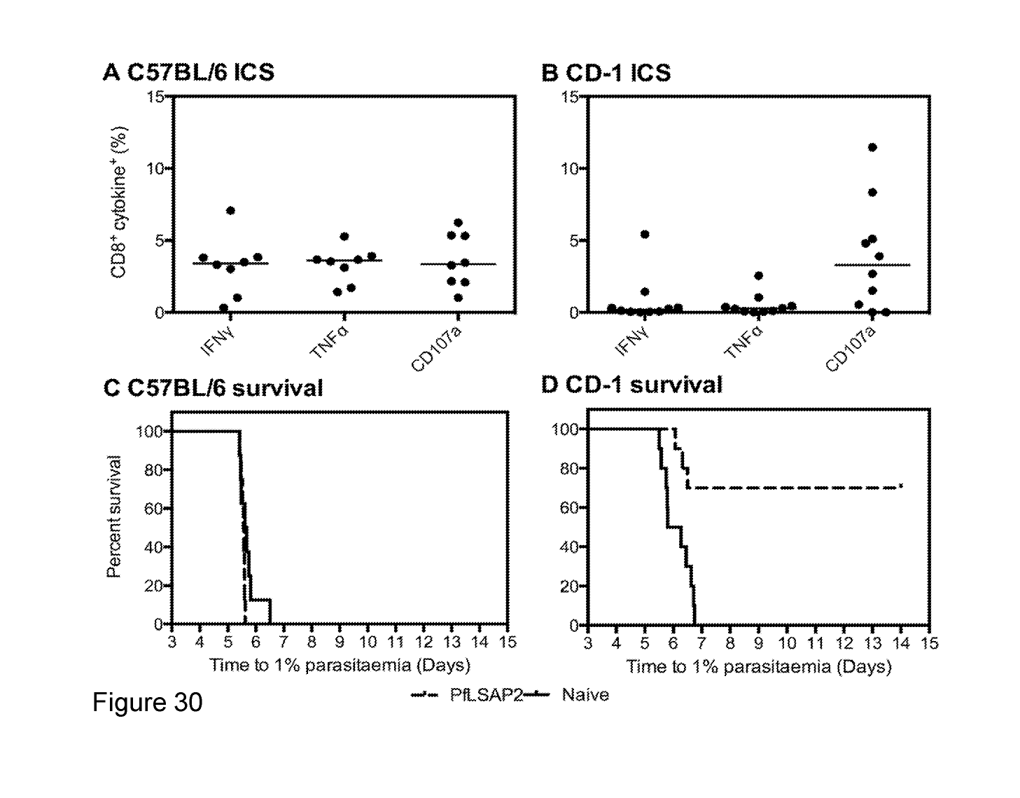

FIG. 30: ChAd63-MVA PfLSAP2 vaccination also provides protection in CD-1 mice, but not C57BL/6. (A and B) CD8+ IFNγ+, TNFγ+ and CD107a+ responses measured in (A) C57BL/6 mice and (B) CD-1 mice three days prior to challenge, expressed as the percentage of total CD8+ cells. Individual data points and the median of eight to ten biological replicates are shown. (C and D) Ten days following ChAd63-MVA vaccination, eight to ten vaccinated mice and eight to ten controls were challenged with 1000 chimeric sporozoites i.v. The Kaplan-Meier curves illustrate the time to 1% parasitaemia, whilst statistical significance between the survival curves was assessed using the Log-Rank (Mantel-Cox) Test. For C57BL/6 (C) p=0.08 and CD-1 (D) p=0.0009.

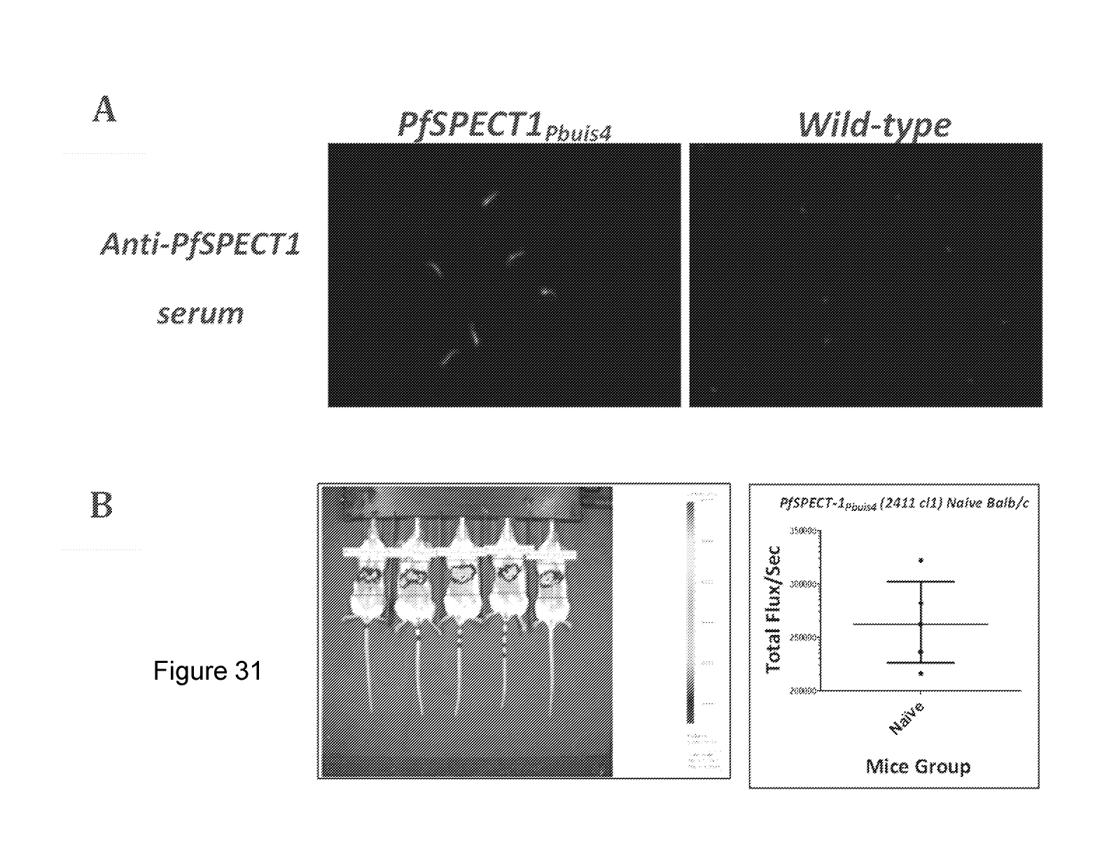

FIG. 31: PfSPECT-1 expressing chimeric parasite phenotype analysis. A. In vivo imaging. Liver loads in naïve mice that were challenged with transgenic chimeric sporozoites were quantified by measuring luminescence levels at 44 hours after infection using the IVIS 200 system. Results are presented as the total flux measured per second. Both median and individual data points are shown. B. Immunofluorescence staining analysis demonstrating PfSPECT-1 antigen expression in sporozoites of chimeric P. berghei parasites. Chimeric salivary-gland sporozoites were stained with sera from vaccinated mice, secondary antibody (Alexa Fluor 488, green) and Hoechst-33342 (blue; nuclear staining). As a control, wild-type (WT) P. berghei sporozoites were stained with the same serum and secondary antibody. Merged images of the different channels are shown for both PfSPECT-1 chimeric parasite and WT P. berghei stained images.

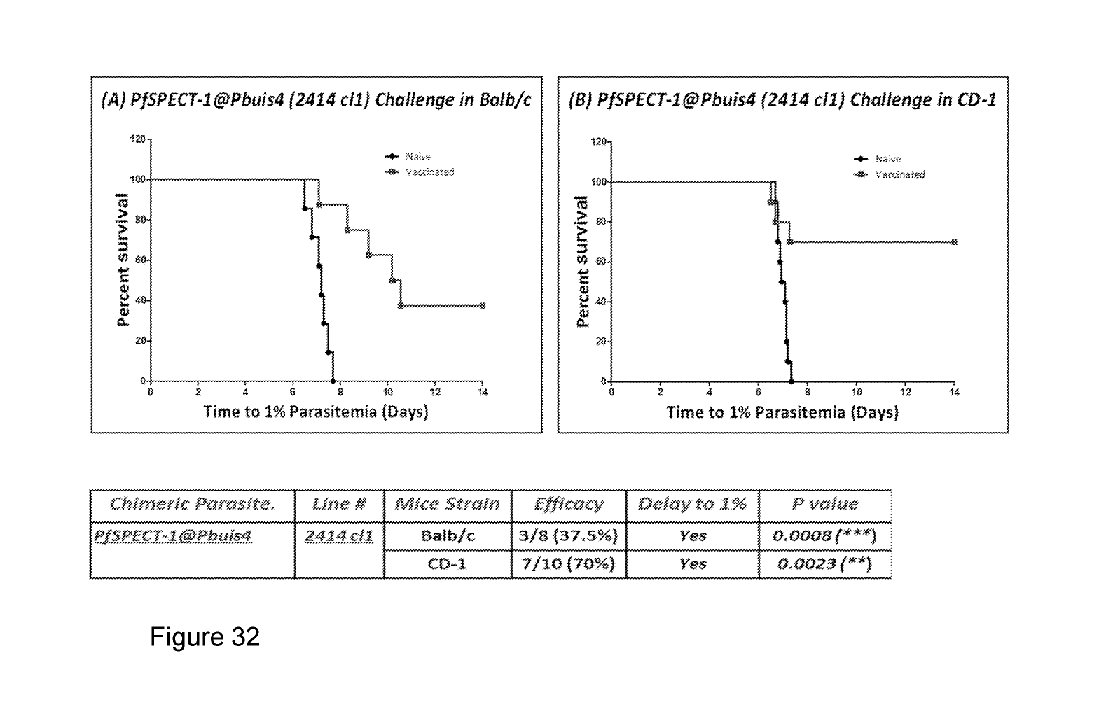

FIG. 32: Confirmation of pre-erythrocytic protection in induced by PfSPECT-1 vaccination in both inbred Balb/c and outbred CD-1 mice. Mice were vaccinated i.m. with 1×10⁸ ifu ChAd63-PfLSPECT-1 followed eight weeks later by 1×10⁷ pfu MVA-PfLSPECT-1. Mice were challenged i.v. with 1000 transgenic PfLSPECT-1_{Pbuis4} (2414 cl1) sporozoites ten days post-MVA boost, along with naïve control mice. Mice were monitored daily to enable calculation of the time to 1% parasitaemia. Mice that were slide-negative at fourteen days post-challenge were considered sterilely protected. The Log-rank (Mantel-Cox) test was used to assess differences between the survival curves. (A) Results from the challenge experiment in Balb/c inbred mice (vaccinated n=8, naive n=8), PfSPECT-1 induced 37.5% sterile protection with a significant delay to 1% parasitaemia p=0.0008. (B) Results from the challenge experiment in CD-1 outbred mice (vaccinated n=10, naive n=10), PfSPECT-1 induced 70% sterile protection with a significant delay to 1% parasitaemia p=0.0023.

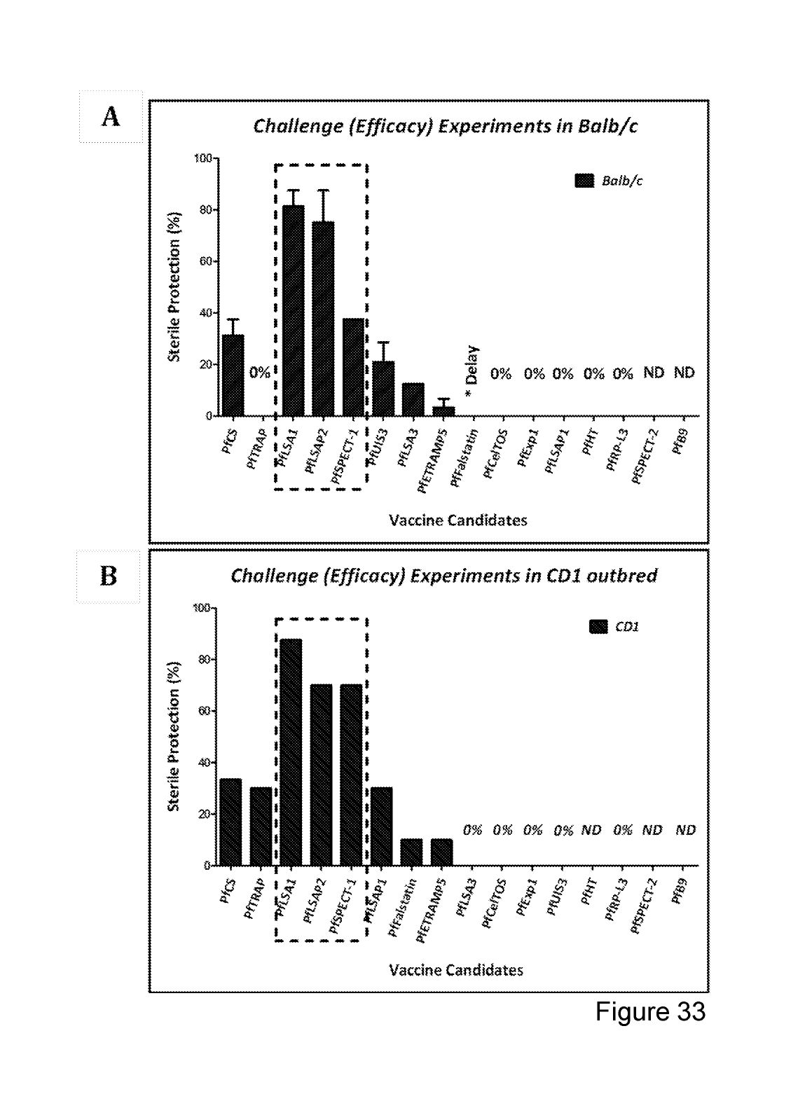

FIG. 33: Overall rank/order showing the protective efficacy of PfSPECT-1 compared to all the assessed P. falciparum vaccine candidates in the same challenge model using chimeric parasites. Screening of 16 novel P. falciparum malaria vaccine candidates using the transgenic malaria challenge model identified three novel promising malaria vaccine candidates (PfLSA1, LSAP-2, and PfSPECT-1) which could induce high level of sterile protection in both (A) Balb/c inbred, and (B) CD-1 outbred mice strains compared to the current leading P. falciaprum malaria vaccines.

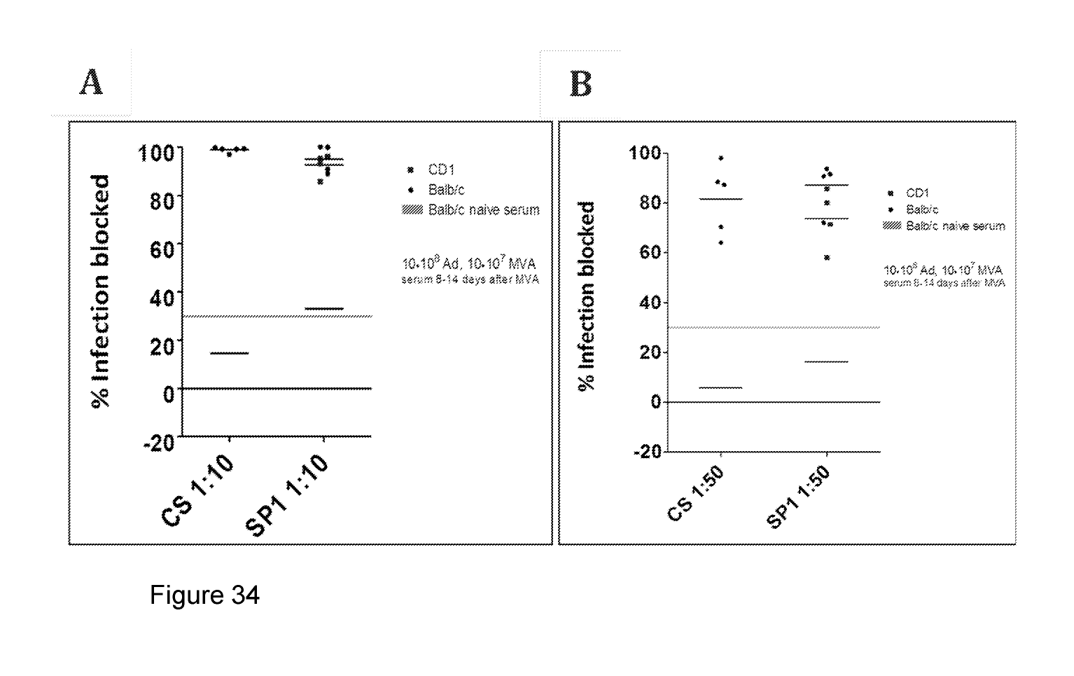

FIG. 34: In vitro assessment of blocking activity of serum from mice vaccinated with PfSPECT-1 viral vaccines. Two different serum concentrations were used 10% and 2% to assess the blocking activity of PfSPECT-1. (A) PfSPECT-1 showed high level of hepatocyte infection blocking; 95% and 93% invasion blocking using 10% serum from Balb/c and CD-1 mice, respectively, in comparison to 99% invasion blocking induced by serum from Balb/c mice vaccinated against PfCSP. (B) While, (A) PfSPECT-1 showed 87% and 74% invasion blocking using 2% serum from Balb/c and CD-1 mice, respectively, in comparison to 81% invasion blocking induced by serum from Balb/c mice vaccinated against PfCSP.

Sequences

PfLSA1 protein sequence with tPA leader underlined - SEQ ID NO: 1
MKRGLCCVLLLCGAVFVSPSQEIHARFRRGMKHILYISFYFILVNLLIFHINGKIIKNS
EKDEIIKSNLRSGSSNSRNRINEEKHEKKHVLSHNSYEKTKNNENNKFFDKDKELTMSN
VKNVSQTNFKSLLRNLGVSENIFLKENKLNKEGKLIEHIINDDDDKKKYIKGQDENRQE
DLEQERLAKEKLQEQQSDLERTKASTETLREQQSRKADTKKNLERKKEHGDVLAEDL
YGRLEIPAIELPSENERGYYIPHQSSLPQDNRGNSRDSKEISIIENTNRESITTNVEGRRDIH
KGHLEEKKDGSIKPEQKEDKSADIQNHTLETVNISDVNDFQISKYEDEISAEYDDSLIDE
EEDDEDLDEFKPIVQYDNFQDEENIGIYKELEDLIEKNENLDDLDEGIEKSSEELSEEKIK
KGKKYEKTKDNNFKPNDKSLYDEHIKKYKNDKQVNKEKEKFIKSLFHIFDGDNEILQIV
DELSEDITKYFMKL
PfLSA1 protein sequence without leader - SEQ ID NO: 2
KHILYISFYFILVNLLIFHINGKIIKNSEKDEIIKSNLRSGSNSRNRINEEKHEKKHVLSHN
SYEKTKNNENNKFFDKDKELTMSNVKNVSQTNFKSLLRNLGVSENIFLKENKLNKEGK
LIEHIINDDDDKKKYIKGQDENRQEDLEQERLAKEKLQEQQSDLERTKASTETLREQQS
RKADTKKNLERKKEHGDVLAEDLYGRLEIPAIELPSENERGYYIPHQSSLPQDNRGNSR
DSKEISIIENTNRESITTNVEGRRDIHKGHLEEKKDGSIKPEQKEDKSADIQNHTLETVNIS
DVNDFQISKYEDEISAEYDDSLIDEEEDDEDLDEFKPIVQYDNFQDEENIGIYKELEDLIE
KNENLDDLDEGIEKSSEELSEEKIKKGKKYEKTKDNNFKPNDKSLYDEHIKKYKNDKQ
VNKEKEKFIKSLFHIFDGDNEILQIVDELSEDITKYFMKL
PfLSA1 nucleic acid sequence - SEQ ID NO: 3
GTACCGCCACCATGAAGCGGGGCCTGTGCTGCGTGCTGCTGCTGTGTGGCGCCGTG
TTCGTGTCCCCCAGCCAGGAAATCCACGCCCGGTTCAGACGGGGCATGAAGCACAT
CCTGTACATCAGCTTCTACTTCATCCTGGTGAACCTGCTGATCTTCCACATCAACGG
CAAGATCATCAAGAACAGCGAGAAGGACGAGATCATTAAGAGCAACCTGCGGAGC
GGCAGCAGCAACAGCCGGAACCGGATCAACGAGGAAAAGCACGAGAAGAAACAC
GTGCTGAGCCACAACAGCTACGAAAAGACCAAGAACAATGAGAACAACAAGTTCT
TCGACAAGGACAAAGAACTGACCATGAGCAACGTGAAGAACGTGTCCCAGACCAA
CTTCAAGAGCCTGCTGCGGAACCTGGGCGTGTCCGAGAACATCTTCCTGAAAGAGA
ACAAGCTGAACAAAGAGGGCAAGCTGATCGAGCACATCATCAACGACGACGACGA
TAAGAAGAAGTACATCAAGGGCCAGGACGAGAACCGGCAGGAAGATCTGGAACAG
GAACGGCTGGCCAAAGAGAAGCTGCAGGAACAGCAGAGCGACCTGGAACGGACCA
AGGCCAGCACCGAGACACTGAGAGAGCAGCAGAGCAGAAAGGCCGACACCAAGA
AGAACCTGGAACGGAAGAAAGAACACGGCGACGTGCTGGCCGAGGACCTGTACGG
CAGACTGGAAATCCCCGCCATCGAGCTGCCCAGCGAGAACGAGCGGGGCTACTAC
ATCCCCCACCAGAGCAGCCTGCCCCAGGACAACCGGGGCAACAGCAGAGACAGCA
AAGAGATCAGCATCATCGAGAACACAAACCGCGAGAGCATCACCACCAACGTGGA
AGGCAGACGGGACATCCACAAGGGCCACCTGGAAGAGAAGAAGGACGGCAGCATC
AAGCCCGAGCAGAAAGAGGACAAGAGCGCCGACATCCAGAACCACACCCTGGAAA
CCGTGAACATCAGCGACGTGAACGACTTCCAGATCTCTAAGTACGAGGATGAGATC
AGCGCCGAGTACGACGACAGCCTGATCGACGAGGAAGAGGACGACGAGGACCTGG
ACGAGTTCAAGCCCATCGTGCAGTACGACAACTTCCAGGACGAGGAAAACATCGG
CATCTACAAAGAGCTGGAAGATCTGATCGAGAAGAACGAGAACCTGGATGATCTG
GACGAGGGCATCGAGAAGTCCAGCGAGGAACTGAGCGAGGAAAAGATCAAGAAG
GGCAAGAAGTACGAGAAAACTAAGGACAACAACTTCAAGCCCAACGACAAGAGCC
TGTACGATGAGCACATCAAGAAGTATAAGAACGACAAACAGGTGAACAAAGAGAA
AGAGAAGTTCATCAAGTCCCTGTTCCACATCTTCGACGGCGACAACGAGATCCTGC
AGATCGTGGATGAGCTGTCCGAGGACATCACCAAGTACTTCATGAAGCTGTGAGC
pfLSAP2 protein sequence - SEQ ID NO: 4
MKRGLCCVLLLCGAVFVSPSQEIHARFRRGMWLCKRGLSVNDTTKCDVPCKD
FYMLFLSNKKEKIKCGTFFGYIFLSKFMKLSISLLLLALIQNILLSNVSLISGSHLYK
RNSRKFAEGYMKGSGSEKNVYLSNKNKEINMNQQSDNKMCDECDDMNQPGDV
NKNDKTSNDQANSSDSDCEPLPFGLKPSDLNRKVTEEDLERMIIELPGKLERKDM
YLIWHYSHSLLRDKFNKMKSSLWSICGKLAHEHKLPFKIKMKKWWKCCGHVTD
ELLIKEHDDYNSIYNYINNESSSREQFLIFLNMIKHSWTTFTMETFIKCKISLENNM
RNVTN
pfLSAP2 protein sequence without leader - SEQ ID NO: 5
WLCKRGLSVNDTTKCDVPCKDFYMLFLSNKKEKIKCGTFFGYIFLSKFMKLSISL
LLLALIQNILLSNVSLISGSHLYKRNSRKFAEGYMKGSGSEKNVYLSNKNKEINM
NQQSDNKMCDECDDMNQPGDVNKNDKTSNDQANSSDSDCEPLPFGLKPSDLNR
KVTEEDLERMIIELPGKLERKDMYLIWHYSHSLLRDKFNKMKSSLWSICGKLAHE
HKLPFKIKMKKWWKCCGHVTDELLIKEHDDYNSIYNYINNESSSREQFLIFLNMI
KHSWTTFTMETFIKCKISLENNMRNVTN
pfLSAP2 nucleic acid sequence - SEQ ID NO: 6
GTACCGCCACCATGAAGCGGGGCCTGTGCTGCGTGCTGCTGCTGTGTGGCGCCGTG
TTCGTGTCCCCCAGCCAGGAAATCCACGCCCGGTTCAGACGGGGCATGTGGCTGTG
CAAGCGGGGCCTGAGCGTGAACGACACCACCAAGTGCGACGTGCCCTGCAAGGAC
TTCTACATGCTGTTTCTGAGCAACAAGAAAGAAAAGATCAAGTGCGGCACCTTCTT
CGGCTACATCTTCCTGAGCAAGTTCATGAAGCTGAGCATCAGCCTGCTGCTGCTGG
CCCTGATCCAGAACATCCTGCTGAGCAACGTGTCCCTGATCAGCGGCAGCCACCTG
TACAAGCGGAACAGCCGGAAGTTCGCCGAGGGCTACATGAAGGGCAGCGGCTCAG
AGAAGAACGTGTACCTGTCCAACAAGAACAAAGAAATCAACATGAACCAGCAGAG
CGACAACAAGATGTGCGACGAGTGTGACGACATGAATCAGCCCGGCGACGTGAAC
AAGAACGACAAGACCAGCAACGACCAGGCCAACAGCAGCGACAGCGACTGCGAGC
CCCTGCCCTTCGGCCTGAAGCCCAGCGACCTGAACCGGAAAGTGACCGAAGAGGA
CCTGGAACGGATGATCATCGAGCTGCCCGGCAAGCTGGAACGGAAGGACATGTAC
CTGATCTGGCACTACAGCCACAGCCTGCTGAGAGACAAGTTCAACAAGATGAAGTC
CAGCCTGTGGTCCATCTGTGGCAAGCTGGCCCACGAGCACAAGCTGCCCTTCAAGA
TCAAGATGAAGAAATGGTGGAAGTGCTGCGGCCACGTGACCGACGAGCTGCTGAT
CAAAGAGCACGACGACTACAACAGCATCTACAACTACATCAACAACGAGTCTAGC
AGCCGCGAGCAGTTCCTGATTTTCCTGAACATGATCAAGCACAGCTGGACCACCTT
CACCATGGAAACCTTCATCAAGTGCAAGATCAGCCTGGAAAACAACATGCGGAAC
GTGACCAACTGAGC
PfUI3 protein sequence - SEQ ID NO: 7
MKVSKLVLFAHIFFIINILCQYICLNASKVNKKGKIAEEKKRKNIKNIDKAIEEHNKRKK
LIYYSLIASGAIASVAAILGLGYYGYKKSREDDLYYNKYLEYRNGEYNIKYQDGAIAST
SEFYIEPEGINKINLNKPIIENKNNVDVSIKRYNNFVDIARLSIQKHFEHLSNDQKDSHVN
NMEYMQKFVQGLQENRNISLSKYQENKAVMDLKYHLQKVYANYLSQEEN
PfUI3 nucleic acid sequence - SEQ ID NO: 8
GTACCGCCACCATGAAGGTGTCCAAGCTGGTGCTGTTCGCCCACATCTTTTTCATCA
TCAACATCCTGTGCCAGTACATCTGCCTGAACGCCAGCAAAGTGAACAAGAAGGGC
AAGATCGCCGAAGAGAAGAAAAGAAAGAACATCAAGAATATCGACAAGGCCATCG
AGGAACACAACAAGCGGAAGAAGCTGATCTACTACAGCCTGATCGCTAGCGGCGC
CATTGCCTCTGTGGCCGCTATCCTGGGCCTGGGCTACTACGGCTACAAGAAAAGCA
GAGAGGACGACCTGTACTACAACAAGTACCTGGAATACCGGAACGGCGAGTACAA
CATCAAGTACCAGGACGGCGCTATCGCCAGCACCAGCGAGTTCTACATCGAGCCCG
AGGGCATCAACAAGATCAACCTGAACAAGCCCATCATCGAGAACAAGAACAACGT
GGACGTGTCCATCAAGCGGTACAACAACTTCGTGGATATCGCCCGGCTGAGCATCC
AGAAGCACTTCGAGCACCTGAGCAACGACCAGAAAGACAGCCACGTGAACAACAT
GGAGTACATGCAGAAATTCGTCCAGGGCCTGCAGGAAAACCGGAACATCAGCCTG
AGCAAGTATCAGGAAAACAAGGCCGTGATGGACCTGAAGTACCATCTGCAGAAGG
TGTACGCCAACTACCTGAGCCAGGAAGAGAACTGAGC
PfI0580c protein sequence - SEQ ID NO: 9
MKRGLCCVLLLCGAVFVSPSQEIHARFRRGMNLLVFFCFFLLSCIVHLSRCSDNNSY
SFEIVNRSTWLNIAERIFKGNAPFNFTIIPYNYVNNSTEENNNKDSVLLISKNLKNSSNPV
DENNHIIDSTKKNTSNNNNNNSNIVGIYESQVHEEKIKEDNTRQDNINKKENEIINNNHQ
IPVSNIFSENIDNNKNYIESNYKSTYNNNPELIHSTDFIGSNNNHTFNFLSRYNNSVLNNM
QGNTKVPGNVPELKARIFSEEENTEVESAENNHTNSLNPNESCDQIIKLGDIINSVNEKIIS
INSTVNNVLCINLDSVNGNGFVWTLLGVHKKKPLIDPSNFPTKRVTQSYVSPDISVTNPV
PIPKNSNTNKDDSINNKQDGSQNNTTTNHFPKPREQLVGGSSMLISKIKPHKPGKYFIVY
SYYRPFDPTRDTNTRIVELNVQ
PfI0580c protein sequence without leader - SEQ ID NO: 10
NLLVFFCFFLLSCIVHLSRCSDNNSYSFEIVNRSTWLNIAERIFKGNAPFNFTIIPYNYVNN
STEENNNKDSVLLISKNLKNSSNPVDENNHIIDSTKKNTSNNNNNNSNIVGIYESQVHEE
KIKEDNTRQDNINKKENEIINNNHQIPVSNIFSENIDNNKNYIESNYKSTYNNNPELIHST
DFIGSNNNHTFNFLSRYNNSVLNNMQGNTKVPGNVPELKARIFSEEENTEVESAENNHT
NSLNPNESCDQIIKLGDIINSVNEKIISINSTVNNVLCINLDSVNGNGFVWTLLGVHKKKP
LIDPSNFPTKRVTQSYVSPDISVTNPVPIPKNSNTNKDDSINNKQDGSQNNTTTNHFPKP
REQLVGGSSMLISKIKPHKPGKYFIVYSYYRPFDPTRDTNTRIVELNVQ
PfI0580c nucleic acid sequence - SEQ ID NO: 11
GTACCGCCACCATGAAGCGGGGCCTGTGCTGCGTGCTGCTGCTGTGTGGCGCCGTG
TTCGTGTCCCCCAGCCAGGAAATCCACGCCCGGTTCAGACGGGGCATGAACCTGCT
GGTGTTCTTCTGCTTCTTCCTGCTGTCCTGCATCGTGCACCTGAGCCGGTGCAGCGA
CAACAACAGCTACAGCTTCGAGATCGTGAACCGGTCCACCTGGCTGAATATCGCCG
AGCGGATCTTCAAGGGCAACGCCCCCTTCAACTTCACCATCATCCCTTACAACTACG
TGAACAACAGCACCGAGGAAAACAACAACAAGGACTCCGTGCTGCTGATCTCCAA
GAACCTGAAGAACAGCAGCAACCCCGTGGACGAGAACAACCACATCATCGACAGC
ACCAAGAAGAACACCTCCAACAACAATAACAACAACTCCAACATCGTGGGCATCT
ACGAGAGCCAGGTGCACGAGGAAAAGATCAAAGAGGACAACACCCGGCAGGACA
ACATCAACAAGAAAGAGAACGAGATCATCAACAACAACCACCAGATCCCCGTGTC
CAACATCTTCAGCGAGAACATCGATAACAACAAGAACTACATCGAGAGCAACTAC
AAGAGCACATACAACAACAATCCCGAGCTGATCCACAGCACCGACTTCATCGGCTC
TAACAACAATCACACCTTCAACTTTCTGAGCCGGTACAACAATAGCGTGCTGAACA
ACATGCAGGGCAACACCAAGGTGCCCGGCAACGTGCCCGAGCTGAAGGCCCGGAT
CTTCTCCGAGGAAGAGAACACCGAGGTCGAAAGCGCCGAAAACAACCACACCAAC
AGCCTGAACCCCAACGAGAGCTGCGACCAGATCATCAAGCTGGGCGACATCATCA
ACAGCGTGAACGAGAAGATCATCAGCATCAACTCCACCGTGAACAACGTGCTGTGC
ATCAACCTGGACTCCGTGAACGGCAACGGCTTCGTGTGGACCCTGCTGGGCGTGCA
CAAGAAGAAGCCCCTGATCGACCCCAGCAACTTCCCCACCAAGAGAGTGACCCAG
AGCTACGTGTCCCCCGACATCAGCGTGACCAACCCCGTGCCCATCCCCAAGAACAG
CAACACCAACAAGGATGACAGCATTAACAACAAGCAGGACGGCAGCCAGAACAAC
ACCACCACCAACCACTTCCCCAAGCCCCGCGAGCAGCTGGTGGGAGGCAGCAGCAT
GCTGATTAGCAAGATCAAGCCCCACAAGCCCGGCAAGTACTTCATCGTGTACAGCT
ACTACCGGCCCTTCGACCCCACCCGGGACACCAACACCCGGATCGTGGAACTGAAC
GTGCAGTGAGC

### 1 MATERIALS AND METHODS

1.1 Materials

1.1.1 Reagents

All commercially available antibodies used are provided in Table 1.1.

TABLE 1.1
Commercially available antibodies used.

| | | Catalogue |
|---|---|---|
| Antibody | Supplier | Number |
| Alexa Fluor ® 488 conjugated goat anti- | Life | A11008 |
| mouse IgG | Technologies | |
| Alexa Fluor ® 488 conjugated goat anti- | Life | A11013 |
| human IgG | Technologies | |
| Anti-human CD8-APC clone OKT8 | eBioscience | 17-0086-73 |
| Anti-human CD3-FITC clone OKT3 | eBioscience | 11-0037 |
| Anti-human CD4-PeCy5.5 clone SK3 | eBioscience | 35-0048-71 |
| Anti-human HLA-A2-FITC clone BB7.2 | Abcam | Ab27728 |
| Anti-human HLA-A2 Purified clone BB7.2 | AbD Serotec | MCA2090EL |
| Anti-human HLA-A3 Purified clone 4i85 | Abcam | Ab33640 |
| Anti-IFNγ blocking antibody AN18 | Mabtech | 3321-3-1000 |
| Anti-mouse CD107a-PE clone 1D4B | eBioscience | 12-1071 |
| Anti-mouse CD11b-Biotin clone M1/70 | BioLegend | 101204 |
| Anti-mouse CD11c-Biotin clone N418 | BioLegend | 117304 |
| Anti-mouse CD127-APCeFluor ® 780 | eBioscience | 47-1271-80 |
| clone A7R34 | | |
| Anti-mouse CD19-Biotin clone MB19-1 | BioLegend | 101504 |
| Anti-mouse CD 16-32 Purified (Fc block) | eBioscience | 14-0161-81 |
| clone 93 | | |
| Anti-mouse CD3ε-APC clone 145-2C11 | eBioscience | 17-0031 |
| Anti-mouse CD45R (B220)-Biotin clone | BioLegend | 103204 |
| RA3-6B2 | | |
| Anti-mouse CD49b-Biotin clone DX5 | BioLegend | 108904 |
| Anti-mouse CD4-Biotin clone GK1.5 | BioLegend | 100404 |
| Anti-mouse CD4-eFluor ® 450 clone RM4-5 | eBioscience | 48-0042-80 |
| Anti-mouse CD4-eFluor ® 650 clone | eBioscience | 95-0041-41 |
| GK1.5 | | |
| Anti-mouse CD4-FITC clone RM4-4 | eBioscience | 11-0043-81 |
| Anti-mouse CD62L-PeCy7 clone MEL-14 | eBioscience | 25-0621-81 |
| Anti-mouse CD8α-FITC clone 53-6.7 | eBioscience | 11-0081 |
| Anti-mouse CD8α-PerCPCy5.5 clone 53- | BD Biosciences | 551162 |
| 6.7 | | |
| Anti-mouse H-2K^{b} Biotin clone AF6-88.5 | BD Biosciences | 553568 |
| Anti-mouse IFNγ-APC clone XMG1.2 | eBioscience | 17-7311 |
| Anti-mouse IFNγ-eFluor ® 450 clone | eBioscience | 48-7311 |
| XMG1.2 | | |
| Anti-mouse IL-2-PeCy7 clone JES6-5H4 | BD Biosciences | 560538 |
| Anti-mouse MHC Class II (I-A/I-E)-Biotin | BioLegend | 107604 |
| clone M5/114.15.2 | | |
| Anti-mouse MHC Class II (I-A/I-E)-PE | eBioscience | 12-5321-82 |
| clone M5/114.15.2 | | |
| Anti-mouse TNFα-FITC clone MP6-XT22 | eBioscience | 11-7321 |
| Anti-TNFα blocking antibody clone | eBioscience | BMS177 |
| 1F3F3D4 | | |

1.1.2 Solutions and Buffers
ACK Lysis Buffer: 8.29 g NH₄Cl (0.15M), 1g KHCO₃ (1 mM), 37.2 mg Na₂EDTA in 800 ml dH₂O. pH adjusted to 7.2-7.4 with HCl (1M) before making a final solution up to 1L with dH₂O.
Buffer A: 50 mM Tris, 100 mM NaCl, 5mM MgCl₂ and 1% Triton X-100 in dH₂O.
Cell Separation Medium: 2% FCS and 1 mM EDTA in D-PBS.
Coating Buffer: 15 mM sodium carbonate and 35 mM sodium bicarbonate capsules were dissolved in dH₂O and autoclaved.
Complete α-MEM Medium: 500 ml MEM α-modification was supplemented with 5 ml L-glutamine (2 mM), 5 ml pen/strep (100 U penicillin, 100 μg streptomycin), 500 μl 2mercaptoethanol (50 μm) and 50 ml of heat inactivated FCS (10%).
Diethanolamine Buffer: A 5× stock was diluted with dH₂O before use.
Digestion Solution: 500 ml DMEM was supplemented with 5 ml L-glutamine (2 mM), 5 ml pen/strep (100 U penicillin, 100 μg streptomycin) and 1.7 g HEPES (15 mM). The solution was filtered prior to use. 1 ml of 250 mg/ml type IV collagenase was added just prior to use.
Ear Punch Buffer: 5 ml 1M Tris pH 8 (50 mM), 40 μl 5M NaCl (2 mM), 2 ml 0.5M EDTA (10 mM) and 10 ml 10% SDS (1%) were added to 82.96 ml dH₂O.
Ex-flagellation Medium: RPMI-1640 was supplemented with 25 mM HEPES, 20% FCS, 10 mM sodium bicarbonate and 50 μm xanthurenic acid. pH was adjusted to 7.6.
FACS Buffer: 1% FCS and 0.1% sodium azide in PBS.
Fructose/PABA Solution: 80 g fructose and 0.5 g PABA were added to 1L of dH₂O. The solution was autoclaved prior to use.
Giemsa: 5% Giemsa in dH₂O.
Hepa1-6 Medium: 500 ml DMEM was supplemented with 5 ml L-glutamine (2 mM), 5 ml pen/strep (100 U penicillin, 100 μg streptomycin), 500 μl mercaptoethanol (50 μm) and 50 ml of heat inactivated FCS (10%).
LB Agar/Broth: Tablets were dissolved in dH₂O (1 tablet per 50 ml). Antibiotics were added at the following working concentrations: Ampicillin 100 μg/ml, Kanamycin 25 μg/ml.
MACS Buffer: 2.5 g BSA (0.5%) and 2 ml 0.5M EDTA (2 mM) were added to 500 ml D-PBS. The buffer was sterile filtered prior to use.
Mowiol: 6 g glycerol and 2.4 g polyvinyl alcohol 4-88 were dissolved in 6 ml dH₂O for two hours at 50° C. with agitation. 12 ml Tris pH 8.5 (0.2M) was added and the solution was dissolved for a further three hours at 50° C. with agitation. The solution was centrifuged at 2500 rpm for five minutes to remove any undissolved solids. DAPI was then added at a final concentration of 0.1 μg/ml.
Perfusion Solution: 5 ml pen/strep (100 U penicillin, 100 μg streptomycin), 2.98 g HEPES (25 mM) and 200 μl0.5M EDTA were added to 500 ml HBSS. The solution was sterile filtered prior to use.
Perm/Wash: 10× Perm/Wash buffer was diluted in dH₂O prior to use.
PBS (0.1M): 0.138M NaCl, 0.0027M KCl, pH 7.4; made by dissolving tablets in dH₂O according to the manufactures instructions.
PBS/Tween (PBS/T) (0.1M): 0.138M NaCl, 0.0027M KCl Tween 0.05%, pH 7.4; made by dissolving sachets in dH₂O.
PBS/BSA: 2.5 g BSA (0.5%) and 250 μl sodium azide (0.05%) were added to 500 ml D-PBS.
Plasmodium berghei Freezing Medium: 11 ml FCS, 4.2 ml 5% NaHCO₃ and 5.5 mg neomycin were added to 96 ml RPMI-1640.
Primary Hepatocyte Culture Medium: 500 ml DMEM was supplemented with 5 ml L-glutamine (2 mM), 5 ml pen/strep (100 U penicillin, 100 μg streptomycin) and 50 ml of heat inactivated FCS (10%).
R0 Medium: 500 ml RPMI-1640 was supplemented with 5 ml L-glutamine (2 mM) and 5 ml pen/strep (100 U penicillin, 100 μg streptomycin).
R10 Medium: 500 ml RPMI-1640 was supplemented with 5 ml L-glutamine (2 mM), 5 ml pen/strep (100 U penicillin, 100 μg streptomycin) and 50 ml of heat inactivated FCS (10%).
TAE Buffer: Made from 50× concentrate diluted in dH₂O.

1.2 Molecular Biology and Cloning

1.2.1 Antigen Inserts

To create the constructs required for cloning into the viral vectors ChAd63 and MVA, the P. falciparum 3D7 sequence was obtained from PlasmoDB (http://plasmodb.org/plasmo/) and cross-referenced with NCBI GenBank (http://www.ncbi.nlm.nih.gov/genbank/). The sequences were analysed using the SignalP 3.0 [1] and TMHMM Servers from the Center for Biological Sequence Analysis (http://www.cbs.dtu.dk/services/) to generate a predicted structure. A number of modifications were made to the original sequences in order to aid production of the virally vectored vaccines, and to increase the insert expression and immunogenicity in mammalian cells. These modifications were the deletion of repetitive regions of sequence and the addition of the human tissue plasminogen activator (tPA) leader sequence (GenBank Accession K03021) [2] upstream. Genes were synthesized by GeneArt (Life Technologies, New York USA), with a number of further modifications requested. The antigen sequence, or tPA leader sequence, was preceded by the Kozak sequence to aid translation in mammalian cells [3] and the Kpn1 restriction enzyme site for cloning into the viral vectors. At the 3′ DNA appendix, a STOP codon and the Not1 restriction enzyme site were added. No sequences contained the Vaccinia virus early gene transcription termination signal 5′-TTTTTNT-3′ [4]. Finally, the sequences were codon optimized for expression in human cells.

P. berghei TRAP (PbTRAP)

The PbTRAP sequence (NCBI AAB63302.1) was synthesized by GeneArt and cloned into the ChAd63 and MVA vectors. The sequence had two modifications, the addition of the tPA leader sequence and removal of the transmembrane domain by addition of two stop codons.

P. falciparum CSP (PfCSP)

The CSP sequence (PlasmoDB PF3D7_0304600) was synthesized by GeneArt and cloned into ChAd63 and MVA vectors [5]. The sequence had two modifications, the addition of the tPA leader sequence and removal of 26 of the NANP repeats from the central region.

P. falciparum ME-TRAP (ME-TRAP)

The ME-TRAP construct has previously been described [6, 7]; the ME string contains known CD4 and CD8 epitopes from pre-erythrocytic P. falciparum antigens and the TRAP sequence is from P. falciparum T9/96 [8]. The ME string was codon optimized for expression in human cells, whilst TRAP was not. Fifteen amino acids were deleted from the T9/96 TRAP sequence (five repeats of PNP) and it contains its own signal peptide. The ME-TRAP construct was cloned into ChAd63 and MVA vectors.

P. falciparum TRAP (TRIP)

The TRIP construct is based on P. falciparum 3D7 TRAP (PlasmoDB PF3D7_1335900). It was codon optimized for expression in human cells, contains the Kozak sequence and also had the same fifteen amino acids deleted as for ME-TRAP. The predicted transmembrane helix and cytoplasmic domains were also deleted. The construct was cloned into ChAd63 and MVA vectors.

Luciferase

The Photinus luciferase gene (NCBI M15077) was sub-cloned from an existing plasmid into ChAd63 and MVA. The gene was confirmed to contain a Kozak sequence and absence of Vaccinia virus early gene transcription termination signals.

MVA-NP+M1

MVA expressing the nucleoprotein (NP) and matrix protein 1 (M1) from Influenza A was generated as previously described [9].

1.2.2 Polymerase Chain Reaction (PCR)

A standard PCR reaction based on KAPA2G Robust Polymerase was used for various applications throughout this study, unless otherwise stated. When PCR products were to be used for down-stream cloning, the Phusion® High-Fidelity DNA Polymerase was used.

1.2.3 Restriction Cloning into ChAd63

The recombinant ChAd63-[antigen] vaccines were constructed using a novel gateway system developed by Dr. Matthew Cottingham at the Jenner Institute, Oxford. This system uses the Gateway® technology to generate a recombinant adenovirus containing the gene of interest under the control of a promoter of choice. To generate such clones, a LR Clonase™ II mediated site-specific recombination occurs between attachment L (attL) sites within an entry vector (containing the gene of interest) and attachment R (attR) sites within the destination vector (the adenovirus genome) (FIG. 1).

The entry vector used was pENTR™ 4-Mono, which contains the human Cytomegalovirus (CMV) immediate-early promoter used to drive transcription and the bovine growth hormone (BGH) poly(A) transcription termination sequence. To avoid deletions during production, this entry vector contains a non-splicing CMV promoter without intron A. The antigen sequences provided by GeneArt, and pENTR™ 4-Mono, were digested with Acc65I and NotI and the resulting DNA fragments were separated on a 1% agarose gel. The DNA bands of correct size were extracted from the gel using Qiagen MinElute extraction kits and the antigen insert was then ligated into the entry vector backbone overnight. The pENTR™ 4-Mono-[antigen] entry vector was then transformed into E. coli bacteria and plasmid DNA prepared. Insert presence was confirmed by analytical restriction enzyme digest using Psi1.

The pENTR™ 4-Mono-[antigen] entry vector was subsequently directionally inserted into the E1 and E3-deleted adenoviral genome at the E1 locus by site-specific recombination using the LR Clonase™ II enzyme mix, as outlined in J160. Reactions were terminated with proteinase K, transformed into E. coli bacteria and plasmid DNA prepared. To confirm insert presence, both analytical restriction enzyme digest using KpnI and sequencing (Gene Service, Oxford) were performed. Following confirmation of the correct sequence the expression clone was linearized with Pme1, prior to transfection and purification.

1.2.4 Restriction Cloning Into MVA

To generate recombinant MVAs the antigen of interest was cloned into the markerless MVA plasmid MVA-GFP-TD (FIG. 1, above). The gene insertion site is at the thymidine kinase (TK) locus with the antigen under control of the p7.5 promoter. The antigen sequences were extracted from the plasmids provided by GeneArt by digestion with Acc65I and NotI. The MVA-GFP-TD plasmid was also digested with the same enzymes, after alkaline phosphatase treatment. The DNA fragments were separated on a 1% agarose gel and extracted using QIAgen MinElute gel extraction kits. The antigen insert was then ligated into the MVA-GFP-TD plasmid overnight. The MVA-GFP-TD-[antigen] vector was then transformed into E. coli bacteria and plasmid DNA prepared. To confirm insert presence, both analytical restriction enzyme digest using PvuI and sequencing (Gene Service, Oxford) were performed. The MVA-GFO-TD-[antigen] vectors were then transfected and purified as outlined in 1.3.2.

1.2.5 Generation of Protein Lysate

In order to detect the presence of antibodies in serum, protein lysate was generated for each of the antigens that were developed into virally vectored vaccines. This entailed In-Fusion® cloning to generate new constructs with the luciferase tag, transfection of HEK293 cells and harvest of the cellular lysate, as detailed below.

1.2.5.1 Generation of pMono2-[Antigen]-rLuc8 Constructs

In order to generate the lysate, a new construct containing the antigen upstream of the Renilla luciferase gene was generated by In-Fusion® cloning of the antigen into a destination plasmid pMono2-FliC-rLuc8. The destination plasmid contained the FliC gene upstream of the luciferase tag. This destination plasmid was digested with HindIII and BamHI to remove the FliC sequence; the DNA fragments were run on a 1% agarose gel and purified using the QIAgen MinElute gel extraction kit.

To obtain insert DNA, PCR primers were designed to cut out the antigen sequence of interest (without tPA leader sequence and STOP codon) from the entry vectors previously generated. These primers also contained fifteen base-pair overhangs matching the entry site of the destination plasmid, containing the HindIII and BamHI restriction sites (Table 1.2). The PCR was performed with Phusion® DNA Polymerase. The PCR insert DNA was then entered into the digested destination vector using the 5× In-Fusion® HD Enzyme Premix according to the manufacturer's instructions, based on a 1:2 insert to vector ratio calculated using the In-Fusion® Molar Ratio Calculator. The resultant product, pMono2-[antigen]-rluc8, was transformed into E. coli bacteria and plasmid DNA prepared. The plasmids were sequenced to confirm correct antigen insert.

TABLE 1.2
Primers used to isolate the liver-stage malaria
antigen sequences from the entry vectors.

| Primer | Sequence |
|---|---|
| PfCe1TOS | GCCAACATGAAGCTTATGAACGCCCTGCGGCGGCTG |
| Forward | CCTGTG |
| PfCe1TOS | CCCGGGCCCGGATCCGTCGAAGAAATCGTCGCTCAG |
| Reverse | GCTTTCCTCGC |
| PFE1590w | GCCAACATGAAGCTTATGCGGTTCAGCAAGGTGTTC |
| Forward | AGC |
| PFE1590w | CCCGGGCCCGGATCCCTGCTCTTTCTTGGGTTCCTCG |
| Reverse | GTTTTC |
| PfExp1 | GCCAACATGAAGCTTATGAAGATCCTGTCCGTGTTCT |
| Forward | TTCTGGCCCTG |
| PfExp1 | CCCGGGCCCGGATCCGTGCTCGGTGCCGGACACCAG |
| Reverse | GTTGTTG |
| PFI0580c | GCCAACATGAAGCTTATGAACCTGCTGGTGTTCTTCT |
| Forward | GC |
| PFI0580c | CCCGGGCCCGGATCCCTGCACGTTCAGTTCCACGAT |
| Reverse | CCG |
| PfLSA1 | GCCAACATGAAGCTTATGAAGCACATCCTGTACATC |
| Forward | AGCTTCTACTTC |
| PfLSA1 | CCCGGGCCCGGATCCCAGCTTCATGAAGTACTTGGT |
| Reverse | GATGTCC |
| PfLSA3 | GCCAACATGAAGCTTATGACCAACAGCAACTACAAG |
| Forward | AGCAACAACAAG |
| PfLSA3 | CCCGGGCCCGGATCCTTTGCTTTTCTGTGTCCGGCTC |
| Reverse | TTTTTTGGC |
| PfLSAP1 | GCCAACATGAAGCTTATGAAGACCATCATCATCGTG |
| Forward | ACCC |
| PfLSAP1 | CCCGGGCCCGGATCCTTCCACCATGTAGAAGTCGGC |
| Reverse | GTCC |
| PfLSAP2 | GCCAACATGAAGCTTATGTGGCTGTGCAAGCGGGGC |
| Forward | CTG |
| PfLSAP2 | CCCGGGCCCGGATCCGTTGGTCACGTTCCGCATGTT |
| Reverse | GTTTTCC |
| PfUIS3 | GCCAACATGAAGCTTATGAAGGTGTCCAAGCTGGTG |
| Forward | CTGTTCG |
| PfUIS3 | CCCGGGCCCGGATCCGTTCTCTTCCTGGCTCAGGTAG |
| Reverse | TTGGCG |

The fifteen base-pair overhangs are highlighted in bold.

1.2.5.2 Transfection of HEK 293A Cells with pMono2-[Antigen]-rLuc8

The transfection reagent was first prepared; 10 μl lipofectamine was mixed with 250 μl Opti-MEM® per sample and incubated for five minutes at room temperature. Meanwhile, 3 μg pMono2-[antigen]-rLuc8 plasmid was mixed with 1 μg green fluorescent protein (GFP) expressing plasmid in 250 μl Opti-MEM®. The DNA and lipofectamine solutions were then mixed together and incubated for twenty minutes at room temperature. 300 μl Opti-MEM® was then added per sample to bring the total volume to 800 μl. The media was then removed from pre-prepared HEK 293A cells in a 6-well plate and the 800 μl mix was added slowly to avoid disturbing the cells. The transfected cells were incubated overnight at 37° C. 5% CO₂ in a humidified incubator. The transfection was then confirmed by the expression of GFP in the cells.

1.2.5.3 Harvest of Cellular Lysate

Lysis buffer provided with the Renilla luciferase assay system was prepared by adding protease inhibitor (100×) immediately prior to harvesting the cellular lysate. The transfected cells were placed on ice and the medium was carefully removed and discarded. 1.4 ml of lysis buffer was added per well and cells were mobilized through the use of a cell scraper. The lysate was transferred into pre-cooled microcentrifuge tubes and sonicated for fifteen seconds. The lysate was then clarified by centrifugation at 12 500 rpm for four minutes. The luciferase activity (light units, LU) of the lysate was quantified on a luminometer (Thermo Scientific Varioskan® Flash) by the addition of 1/100 Renilla luciferase assay substrate.

1.2.6 Genotyping of HHD Mice

To determine the genotype of the HLA-A2 transgenic mice bred in-house, known as HHDs [10], ear punches were collected in sterile microcentrifuge tubes. To extract DNA, 20 μl of ear punch buffer containing 1 mg/ml proteinase K was added to each ear punch and incubated for twenty minutes at 55° C. The sample was then vortexed to help break up the tissue, followed by a further twenty minutes of incubation. 180 μl dH₂O was then added to each tube and samples were heated to 99° C. for five minutes to deactivate the proteinase K. After cooling samples were stored at −20° C. until further use. PCR was then performed.

Primers were designed for HLA-A2, H-2D, human and mouse beta-2 microglobulin (β2m) (Table 1.3). Control DNA was collected from the HepG2 cell line (HLA-A2) and C57BL/6 mice (H-2D^{b}). HHD mice should contain human β32m, human HLA-A2 (α1 and α2 domains) and mouse H-2D^{b} (α3, transmembrane and cytoplasmic domains). However, the genotyping results indicated that whilst they do contain HLA-A2, they actually contain mouse β2m and not human β2m. Flow cytometry staining confirmed lack of expression of H-2^{b} compared to C57BL/6 mice, and a low level expression of HLA-A2 using the antibodies to H-2K^{b} (AF6.88.5.5.3) and HLA-A2 (BB7.2). This also confirmed the finding that HHD mice contain mouse rather than human β2m, as β2m is essential for cell surface expression of MHC molecules. Nevertheless, these mice were able to generate HLA-A2 specific responses with an Influenza A HLA-A2-restricted epitope.

TABLE 1.3
Primers used to genotype HHD mice.

| | | Product |
|---|---|---|
| Primer Name | Sequence | Size |
| H-2D^{b} Forward | GCGGAGAATCCGAGATATGA | 157 bp |
| H-2D^{b} Reverse | CCGCGCTCTGGTTGTAGTAG | |
| HLA-A2 Forward | ACCGTCCAGAGGATGTATGG | 202 bp |
| HLA-A2 Reverse | CCAGGTAGGCTCTCAACTGC | |
| Human β2m Forward | TGGCACCTGCTGAGATACTG | 713 bp |
| Human β2m Reverse | CAGTTCCTTTGCCCTCTCTG | |
| Mouse β2m Forward | CTTGGACCCTTGGTACCTCA | 249 bp |
| Mouse β2m Reverse | AAGTCCAGTGTTGGGTCAGG | |

1.3 Virology

1.3.1 Adenovirus Transfection and Purification

85 μl linearised recombinant adenoviral plasmid was mixed with 215 μl Opti-MEM®. 300 μl of 1:10 lipofectamine in Opti-MEM® was then prepared and mixed with the 300 μl of linearised plasmid, followed by incubation of the resulting mixture for at least twenty minutes at room temperature. The mixture was then added to flasks of pre-prepared T-REx™ 293 cells. 293 cells are immortalized lines of primary human embryonic kidney cells transformed by sheared human adenovirus 5 DNA. They therefore provide the E1 gene product, in trans, for the replication-incompetent adenovirus. Cells were incubated at 37° C. 5% CO₂ in a humidified incubator and monitored daily for cytopathic effect (CPE, morphological changes caused by virus infection). Cells were harvested once optimal CPE was evident. Recombinant adenovirus was purified by density centrifugation over a caesium chloride gradient. Virus yield (infectious units) was determined by plaque immunostaining.

1.3.2 MVA Transfection and Purification

Antigens were cloned into the markerless MVA plasmid (MVA-TD-GFP) where the GFP gene is present outside the TK locus. Chick Embryo Fibroblasts (CEFs) (obtained from the Pirbright Institute, Compton, UK) were maintained and infected with MVA expressing red fluorescent protein (RFP). These cells were then transfected with the MVA-TD-GFP-[antigen] plasmid 90 minutes later, which enables homologous recombination to occur between the MVA virus and the plasmid. As the plasmid is circular, a single crossover event occurs resulting in a large unstable intermediate product containing the entire plasmid and MVA parental genome. This unstable product then resolves into either the recombinant markerless MVA or the parental MVA containing RFP. After incubation of the plasmid with the virus in CEFs, cells were sorted using a MoFlo cell sorter. The unstable intermediate products expressing both GFP and RFP were collected and the lysate used to infect CEFs again. Successful recombinant MVAs containing the antigen were selected by repeated rounds of plaque picking, initially selecting GFP and RFP double positive cells followed later by the selection of colourless plaques. The virus was then bulked up and purified, followed by PCR analysis and titration (pfu).

1.4 Animals and Immunisations

1.4.1 Mice

All procedures were carried out according to the UK Animals (Scientific Procedures) Act 1986 and approved by the University of Oxford Animal Care and Ethical Review Committee for use under Project License PPL 30/2414 or 30/2889. All mice were housed under Specific Pathogen Free (SPF) conditions, in the Wellcome Trust Centre for Human Genetics Animal Facility, or temporarily in the Radiobiology Research Institute when used in imaging studies.

Five to six week old female C57BL/6J (H-2^{b}), Balb/c (H-2^{d}), TO (outbred) or CD-1 (outbred) mice were obtained from Harlan (UK). HHD (HLA-A2 transgenic) mice [10] were kindly provided by Professor Vincenzo Cerundolo (University of Oxford) and bred in the FGF by the facility's staff.

1.4.2 Immunisations and Injections

All immunisations were carried out under inhalation anaesthesia, using 3.5% isoflurane carried by oxygen (2 L/min). Immunisations were administered intramuscular (i.m.) in a volume of 50 μl into the musculus tibialis using 26-gauge needles.

Intravenous (i.v.) injections were administered in a volume of 100 μl into the lateral tail vein using a 28-gauge needle. Prior to injection, mice were warmed for approximately ten minutes at 38° C. to encourage vasodilation.

Intraperitoneal (i.p.) injections were administered in a volume of 100-300 μl using a 28-gauge needle.

Subcutaneous (s.c.) injections were administered into the scruff of the neck in a volume of 50 μl using 26-gauge needles.

1.4.3 Vaccines

All vaccines were formulated in endotoxin free D-PBS to a total volume of 50 μl per mouse and administered i.m. Adenoviral vectored vaccines were given at a dose of 1×10⁶ or 1×10⁸ infectious units (ifu), whilst MVA vectored vaccines were given at either 1×10⁶ or 1×10⁷ plaque forming units (pfu) as stated in the relevant text and figure legends.

1.4.4 Isolation of Splenocytes

Mice were sacrificed by cervical dislocation and spleens were dissected and removed into sterile D-PBS. Individual spleens were subsequently crushed in 5 ml PBS using the flat end of a 5 ml syringe in a 6-well plate. Single cell suspensions were prepared by passaging splenocytes through a 70 μm cell strainer into a 50 ml tube prior to centrifugation at 1350 rpm for five minutes. To remove erythrocytes, supernatants were discarded and cell pellets resuspended in 5 ml ACK lysis buffer for four minutes before addition of 25 ml PBS to stop the reaction. Splenocytes were immediately centrifuged again and the resulting cell pellets resuspended in 5 ml complete α-MEM. Splenocytes were counted using a CASY counter (Scharfe Systems, Germany) and diluted to the required concentration in complete α-MEM.

1.4.5 Isolation of Peripheral Blood Mononuclear Cells (PBMCs)

Five to six drops of blood were collected from the lateral tail vein into 200 μl 10 mM EDTA in PBS. Prior to bleeding mice were warmed for approximately ten minutes at 38° C. to encourage vasodilation. Approximately 1 ml of ACK lysis buffer was added to the blood, followed immediately by thorough vortexing and centrifugation at 4000 rpm for four minutes. The cell pellet was resuspended in 1 ml ACK lysis buffer and again centrifuged prior to resuspending the pellet in 320 μl complete α-MEM.

1.4.6 Isolation of Liver Mononuclear Cells

Mice were sacrificed by cervical dislocation and the liver was exposed. A 25-gauge butterfly needle attached to a 50 ml syringe was used to flush the circulating blood from the liver with sterile D-PBS, by insertion into the hepatic portal vein. The liver was subsequently dissected and mashed through a 70 μm cell strainer into a petri dish, with the flat end of a 2 ml syringe. The cell strainer and petri dish were flushed with PBS and all cells were collected into a 15 ml tube. The cells were centrifuged for seven minutes at 1500 rpm, the supernatant discarded and the cell pellet resuspended in 10 ml of 33% isotonic percoll solution. The cells were then centrifuged at 693×g for twelve minutes with the brakes off. The resulting upper layers were carefully removed with a transfer pipette and the cell pellet was resuspended in 1 ml of ACK lysis buffer. The cells were incubated in the lysis buffer for four minutes at room temperature then 10 ml complete α-MEM was added and the cells were spun for five minutes at 1500 rpm. The final pellet was resuspended in 500 μl complete α-MEM.

1.4.7 Isolation and Culture of Primary Murine Hepatocytes

The isolation and culture of primary murine hepatocytes was based on a procedure described by Prof. David Tosh at the University of Bath [14], with various modifications to suit the technical set-up available at the University of Oxford. To comply with the project license, mice were sacrificed by cervical dislocation prior to the procedure commencing. Mice were quickly dissected to expose the liver, moving all other organs to the side. An 18-gauge catheter was inserted into the vena cava, the needle removed and tubing connected to the solutions attached. The hepatic portal vein was cut; instantaneous blanching of the liver indicated successful insertion of the cannula. The liver was then perfused for ten minutes with the perfusion solution kept at 37° C. in a water bath and delivered at a constant rate (approximately 5 ml/minute) through the use of a mechanical pump. Following adequate perfusion, the liver was digested at ten minutes with a constant rate of digestion solution at 37° C.

Subsequently, the liver was carefully dissected from the mouse and removed into a petri dish containing digest solution. The liver was gently teased apart with a pair of forceps, releasing the cells into the dish. The cell suspension was passed through a 70 μm strainer into a 50 ml tube. The cell suspension was then spun three times at 50×g for two minutes, with resuspension in primary hepatocyte culture medium. Cells were diluted in trypan blue to determine the number and viability of cells using a haemocytometer. Cells were resuspended at 5×10⁶ cells/ml and 100 μl were added per well of a 96-well collagen coated plate.

1.4.8 Collection of Mouse Sera

Mouse sera was obtained from either five to six drops of blood from the lateral tail vein collected in a microvette tube, or via cardiac puncture. Cardiac puncture was performed under anaesthetic (3.5% isoflurane, 2 L/minute oxygen), using a 26-gauge needle to withdraw blood from the heart. Collected blood was stored at 4° C. overnight to allow clotting. The following day blood was spun at 13 500 rpm for four minutes to separate the sera from the RBCs. Sera was removed into a clean microcentrifuge tube and stored at −20° C. until required.

1.5 Immunological Assays

1.5.1 Peptides

Peptides used in the cellular assays were commercially synthesized by Neo Group Inc., USA, Mimotopes, UK or Thermo Fisher Scientific, USA. Crude 20mer peptides overlapping by ten amino acids were synthesized for the entire sequence used in the vaccine constructs for: P. falciparum 3D7 CSP, Expl, LSA1, LSA3, LSAP1, LSAP2, PFE1590w, PFI0580c, TRAP and UIS3, P. falciparum T9/96 TRAP and P. berghei TRAP. Crude 15mer peptides overlapping by ten amino acids were synthesized for the entire sequence of P. falciparum 3D7 AMA1, Ce1TOS, MSP1, MSP2, Pfs16 and STARP. Crude 20mer peptides overlapping by ten amino acids for the Influenza A NP and M1 antigens, including the HLA-A2-restricted epitope in M1, were kindly provided by Dr. Teresa Lambe (University of Oxford). Peptides were reconstituted in DMSO at a concentration of 50 to 100 mg/ml depending on the solubility. Peptides were subsequently combined into sub-pools of up to twenty peptides, before the sub-pools were combined into a total mega-pool for use in the cellular assays. These peptides were used for both murine and human cellular assays.

Intracellular Cytokine Staining (ICS)

Cellular immune responses were assayed in splenocytes, PBMCs and liver mononuclear cells via ICS. Isolated cells were plated at 150 μl cells with 50 μl stimulated (+peptide) or unstimulated (−peptide) mixes in a 96-well U bottom plate for six hours at 37° C. 5% CO₂ in a humidified incubator. Mixes contained 1/1000 Brefeldin A (golgi plug) per well, 1/400 anti-mouse CD107a-PE+/−5 μg/ml peptide (final concentrations) in complete α-MEM. Plates were then stored at 4° C. overnight or stained that day.

Plates were centrifuged at 1800 rpm for three minutes to pellet cells, which were then washed in 100 μl PBS/BSA and centrifuged again. For standard ICS, cells were then surface stained with 50 μl per well of 1/50 anti-mouse CD16/32 (Fc block), 1/100 anti-mouse CD4-eFluor® 450 and 1/200 anti-mouse CD8α-PerCPCy5.5 diluted in PBS/BSA for 30 minutes at 4° C. Cells were subsequently washed once and fixed by incubation for five minutes at 4° C. with 4% paraformaldehyde (10% neutral buffered formalin). Cells were washed once in Perm/Wash followed by intracellular staining with 50 μl per well of 1/100 anti-mouse TNFα-FITC, 1/100 anti-mouse IL-2-PeCy7 and 1/200 anti-mouse IFNγ-APC diluted in Perm/Wash for 30 minutes at 4° C. Finally, cells were washed three times in Perm/Wash and once in PBS/BSA with final resuspension in 80 μl PBS/BSA.

To stain for memory cell markers, the first layer compromised 1/50 anti-mouse CD16/32 (Fc block), 1/200 anti-mouse CD8α-PerCPCy5.5, 1/50 anti-mouse CD4-eFluor® 650, 1/50 anti-mouse CD621-PeCy7, 1/50 anti-mouse CD127-APCeFluor® 780 and 1/200 Live/Dead Aqua diluted in PBS/BSA. The second layer compromised 1/100 anti-mouse TNFα-FITC and 1/100 anti-mouse IFNγ-eFluor® 450 diluted in PBS/BSA. All other steps were identical as for the standard ICS detailed above.

Samples were acquired on a LSRII (BD Biosciences) flow cytometer and analysis was performed using FlowJo (Tree Star Inc., USA). Splenocytes, liver mononuclear cells or PBMCs were first gated by size, followed by singlet cells. The cells were then separated into CD4 or CD8 positive subsets, and then cytokines gated from within those subsets. Gates show the percentage of the parent. Background responses in unstimulated wells were subtracted from the stimulated responses. In some experiments polyfunctionality of T cells was analysed using the Boolean gate platform in FlowJo followed by subsequent preparation of data in Pestle (Mario Roederer, National Institutes of Health) for final analysis and graphical representation in SPICE (simplified presentation of incredibly complex evaluations, Mario Roederer [17]).

1.5.2 Mouse Ex-Vivo Spleen IFNγ Enzyme-Linked Immunosorbent Spot (ELISpot) Assay

All ELISpot reagents were supplied in a mouse IFNγ ELISpot kit from Mabtech. ELISpot plates were coated with 50 μl per well of 5 μg/ml anti-IFNγ purified monoclonal antibody AN18 in carbonate-bicarbonate buffer and incubated at 4° C. overnight. Plates were then blocked for at least one hour at room temperature with 100 μl complete γ-MEM. Mouse splenocytes were prepared and diluted to an optimal starting concentration (most commonly 10×10⁶ cells/ml, dependent on expected/observed response). 50 μl splenocytes were added per well in duplicate and serially diluted two-fold down the blocked plates. Peptides were diluted to 2 μg/ml and 50 μl was added per test well (final concentration of 1 μg/ml); complete α-MEM alone was added to control wells. Plates were incubated for eighteen to twenty hours at 37° C. 5% CO₂ in a humidified incubator.

Following incubation, plates were washed six times with PBS using an automated plate washer (Dynex Technologies, USA) then incubated with 50 μl per well of 1 μg/ml biotinylated rat anti-mouse IFNγ diluted in PBS for two hours at room temperature. Plates were subsequently washed again and incubated with 50 μl per well of 1 82 g/ml streptavidin alkaline phosphatase polymer diluted in PBS for one hour at room temperature. Plates were washed again and finally incubated with 50 μl per well of BioRad AP conjugate development buffer for approximately five to ten minutes at room temperature until spots developed. Washing the plates with tap water stopped the reaction and once plates were dry spots were enumerated using an AID ELISpot plate counter (Strassberg, Germany). Responses were expressed as spot forming units (SFU) per million splenocytes. Background responses in media-only wells were subtracted from those measured in peptide-stimulated wells.

1.5.3 Isolation and Adoptive Transfer of CD4⁺ and CD8⁺ T Cells

Splenocytes were prepared and counted followed by sequential isolation of CD4⁺ then CD8⁺ T cells, using the MACs CD4 (L3T4) MicroBeads (positive selection) and CD8⁺ T Cell Isolation Kit (negative selection) as per the manufacturer's instructions. All centrifugation steps were performed at 4° C., all incubation steps at 2-8° C. and all solutions used were pre-cooled.

1.5.3.1 Positive Selection of CD4⁺ T Cells

Briefly, splenocytes were centrifuged at 300×g for ten minutes then resuspended in 90 μl MACS buffer and 3.5 μl CD4 (L3T4) MicroBeads per 10⁷ cells. Samples were mixed well then incubated for fifteen minutes followed by washing in 1-2ml MACS buffer per 10⁷ cells and centrifugation at 1500 rpm for eight minutes. Cells were resuspended in 500 μl MACS buffer for up to 10⁸ cells and separated using a MACS Separator and LS Column. The column was prepared by placing within the magnet and rinsing with 3 ml MACS buffer. The cell suspension was then applied to the column and washed through three times with 3 ml MACS buffer; the collected effluent was the unlabelled fraction. The column was removed from the Separator and placed on a 15 ml tube; 5 ml MACS buffer was added and the labelled cells were flushed out by firmly applying the provided plunger. The positive fraction (CD4⁺ T cells) was set-aside on ice and the unlabelled fraction was used to isolate CD8⁺ T cells.

1.5.3.2 Negative Selection of CD8⁺ T cells

Briefly, the unlabelled fraction from the CD4⁺ selection was centrifuged at 300×g for ten minutes then resuspended in 40 μl MACS buffer and 2.8 μl Biotin-Antibody cocktail per 10⁷ cells. The Biotin-Antibody cocktail contained monoclonal antibodies (mAbs) against CD4, CD11b, CD11c, CD19, CD45R (B220), CD49b (DX5), CD105, MHC Class II and Ter-119. Samples were mixed well then incubated for ten minutes, followed by addition of 30 μl MACS buffer and 5.7 μl Anti-Biotin MicroBeads per 10⁷ cells and further incubation for fifteen minutes. Cells were then washed in 1-2 ml MACS buffer per 10⁷ cells and centrifuged at 1500 rpm for eight minutes. Cells were resuspended in 500 μl MACS buffer for up to 10⁸ cells and separated using a MACS Separator and LS Column. The column was prepared by placing within the magnet and rinsing with 3 ml MACS buffer. The cell suspension was then applied to the column and washed through three times with 3 ml MACS buffer; the collected effluent was the unlabelled fraction containing the CD8⁺ T cells.

The CD4⁺ and CD8⁺ T cell fractions were centrifuged and cell numbers determined. For injection into mice, the cells were again centrifuged and resuspended in RPMI-1640 with 10% FCS at the required concentration. The cells were injected i.v. in 100 μl two days prior to challenge with Plasmodium parasites. The purity of the fractions was analysed by flow cytometry using anti-CD4-eFlour® 450, anti-CD8-PerCPCy5.5 and anti-CD3ε-APC.

1.5.4 Enrichment of CD8⁺ T cells

Splenocytes were prepared and counted, then enriched for CD8⁺ cells by negative depletion using an in-house biotin-antibody cocktail and MACS anti-Biotin MicroBeads. Briefly, splenocytes were centrifuged at 300×g for ten minutes then resuspended in 40 μl MACS buffer and 1 μl biotin-antibody cocktail per 10⁷ cells. The biotin-antibody cocktail contained mAbs against CD4, CD11b, CD11c, CD19, CD45R (B220), CD49b (DX5) and MHC Class II diluted 1/00 in MACS buffer and sterile filtered. Samples were mixed well then incubated for ten minutes, followed by addition of 30 μl MACS buffer and 20 μl Anti-Biotin MicroBeads per 10⁷ cells and further incubation for fifteen minutes. Cells were then washed in 1-2 ml MACS buffer per 10⁷ cells and centrifuged at 300×g for ten minutes. Cells were resuspended in 500 μl MACS buffer for up to 10⁸ cells and separated using a MACS Separator and LD Column. The column was prepared by placing within the magnet and rinsing with 2 ml MACS buffer. The cell suspension was then applied to the column and washed through twice with 1 ml MACS buffer; the collected effluent was the unlabelled fraction containing the CD8⁺ T cells. The column was removed from the Separator and placed on a 15 ml tube; 3ml MACS buffer was added and the labelled cells were flushed out by firmly applying the provided plunger. The purity of the fractions was analysed by flow cytometry by staining with 1/100 anti-CD8α-FITC.

1.5.5 In vivo CD4⁺ or CD8⁺ T Cell Depletion

To determine the contribution of T cells in protection from malaria, subsets of T cells were depleted using the monoclonal antibodies anti-CD4 GK1.5 (rat IgG2a) or anti-CD8 2.43 (rat IgG2a) purified using protein G affinity chromatography from hybridoma culture supernatants. IgG from normal rat serum was purchased and purified using the same method. The optimal dose of depleting mAbs was determined experimentally as 100 μg by dose titration.

Mice were injected i.p. with 100 μg of mAb diluted in PBS on days −2, −1 and 0 (with respect to challenge with Plasmodium parasites on day 0). Control mice were treated in the same way. The degree of in vivo CD4⁺ or CD8⁺ T cell depletion was assessed by flow cytometry using 1/100 anti-CD4-FITC clone RM4-4, 1/200 anti-CD8-PerCPCy5.5 clone 53-6.7 and 1/50 anti-CD3ε-APC on day +4 with respect to day of challenge.

1.5.6 Luminescence Immunoprecipitation Assay (LIPS)

The LIPS assay was used to detect antigen-specific antibody in sera from immunized subjects. Burbelo and colleagues developed this assay in 2005 [18, 19]; it is useful when purified recombinant proteins needed for ELISA are not available. The assay relies on the generation of plasmid constructs containing the antigen of interest fused to the Renilla luciferase sequence. These plasmids are subsequently transiently transfected into cells and the cellular lysate harvested.

50 μl of 1/100 sera diluted in Buffer A was mixed with 50 μl of 2×10⁸ LU/ml of [antigen]-rluc8 lysate in a 96-well V bottom plate for one hour at room temperature on a rotary shaker. A 30% suspension of protein A/G beads in PBS was prepared, 5 μl was added per well to a 96-well filter MultiScreen HTS plate and the 100 μl sera-lysate mix was transferred to this plate and incubated for a further hour at room temperature on a rotary shaker. Plates were developed using the Promega Renilla luciferase assay system. The plates were first washed eight times with 100 μl Buffer A, followed two times with PBS and finally left in 50 μl PBS to prevent the membrane from drying out. A 1/100 dilution of Renilla luciferase assay substrate was prepared in the provided buffer and 50 μl was added per well. Plates were read immediately on a luminometer (Thermo Scientific Varioskan® Flash) and each well was subsequently quenched with 2 M HC1 to prevent cross talk between wells. The background level of luminescence was calculated using six replicates of naïve sera: two times the standard deviation plus the average. Where available, positive control sera or monoclonal antibodies were also included. The LIPS was validated by correlation with ELISA readings for the antigens PfTRAP and PfCe1TOS (Spearman r=0.7115, p<0.001 and r=0.61, p=0.0043, respectively).

1.5.7 Enzyme-Linked Immunosorbent Assay (ELISA)

ELISA was performed to detect antibodies when recombinant purified protein was available, to provide a measure to test the accuracy of the LIPS assay. PfCe1TOS protein was obtained from Dr. Matt Higgins (Biochemistry, University of Oxford) to perform PfCe1TOS ELISAs on sera from vaccinated mice.

NUNC Maxisorp 96-well flat bottom plates were coated with 50 82 l per well of 2 μg/ml PfCe1TOS protein diluted in carbonate-bicarbonate buffer and incubated at 4° C. overnight. Plates were washed six times with PBS-0.05% Tween (PBS/T) then blocked with 200 μl 1% BSA in PBS/T per well for one hour at 37° C. Serum samples taken after a single shot of ChAd63-[antigen] were diluted 1/100 in PBS/T, samples taken after ChAd63-[antigen] with MVA-[antigen] boost were diluted 1/500. Samples were added to wells in duplicate and serially diluted three-fold down the plate. Plates were incubated for two hours at room temperature then washed six times with PBS/T.

Bound antibodies were detected by the addition of 50 μl per well of 1/5000 goat anti-mouse whole IgG alkaline phosphatase conjugate diluted in PBS/T and incubated for one hour at room temperature. Plates were washed six times in PBS/T then developed with 100 μl per well of1 mg/ml 4-Nitrophenyl phosphate disodium salt hexahydrate in diethanolamine buffer. Plates were read when the positive controls gave an optical density (OD)₄₀₅ of approximately one. The endpoint titres were taken as the dilution at which the OD of the sample reached the background plus three times the standard deviation calculated from naïve samples.

1.5.8 Whole IgG Passive Transfer

Serum was collected from anaesthetized mice as previously described in 1.4.8. Sera were pooled between groups and IgG purified using Pierce polypropylene columns pre-packed with 2 ml protein G resin as per the manufacturer's instructions. Approximately 1.5 mg of purified whole IgG was obtained, and 173 μg was injected i.v. in 100 μl into each naive mouse. Those mice were subsequently challenged with malaria sporozoites approximately six hours later.

1.6 Parasitology

1.6.1 Parasite Strains

Plasmodium parasite strains were provided by collaborators, as detailed below.

P. berghei ANKA GFP (Wild-type expressing GFP—referred to as P. berghei GFP herein) was provided by Prof. Robert Sinden at Imperial College, London [20].

P. berghei transgenic parasites containing an additional copy of the P. falciparum version of a particular gene inserted at the 230 p locus under control of the P. berghei UIS4 promoter were provided by Leiden University, the Netherlands. All of these parasites also expressed a GFP/luciferase fusion gene under the P. berghei EF 1α promoter. Generation was through the ‘gene insertion/marker out’ technology, as previously described [21]. Transgenic parasites were generated for the following P. falciparum antigens: Ce1TOS, LSA1, LSA3, LSAP1, LSAP2, UIS3, PFI0580c, PFE1590w, TRAP and CSP. In this study, they are referred to as PbPf[antigen], for example, PbPfCe1TOS.

P. falciparum 3D7 was provided by Walter Reed Army Institute of Research (WRAIR), USA, and P. falciparum NF54 by Radboud University Nijmegen, the Netherlands.

1.6.2 Preparation of Thin Blood Smears

To monitor parasitaemia of infected mice, thin blood smears were prepared by snipping the end of the mouse's tail and collecting a single drop of blood onto a glass slide. The smear was air-dried, fixed in 100% methanol for one minute then stained in 5-10% Giemsa diluted in dH₂O for one hour. The slide was viewed on a light microscope at 100× under oil immersion. The percentage of parasitized red blood cells (pRBCs) was counted at a monolayer region of the thin blood smear, where there were always approximately 500 RBCs per field of view. The number of fields of view counted depended on the parasitaemia. If the parasitaemia was above 1% five fields of view were counted, if it was between 0.1% and 1% ten fields of view were counted and if it was below 0.1% 40 fields of view were counted.

1.6.3 Sporozoite Production (P. berghei)

Frozen P. berghei pRBC were thawed and 100-30 μl was injected i.p. into a naïve TO donor mouse. Four days later the parasitaemia of the donor mouse was analysed. The donor mouse was then cardiac bled, however in this case the syringe was lined with 300 U/ml heparin to prevent the blood clotting. The blood was diluted to 1% parasitaemia and 100 μl was injected i.p. into two recipient mice. This equates to approximately 10⁷ pRBCs injected into each recipient mouse. Three days after the recipient mice had been inoculated they were anaesthetized with 50-100 μl i.m. of a mix of 2% Rompun solution (20 mg/ml xylazine), 100mg/ml Ketaset (ketamine) and PBS in a ratio of 1:2:3 and fed to a pot of starved 4-7 day old female Anopheles stephensi mosquitos for approximately ten minutes. During the feed a drop of blood was taken to determine parasitaemia, and another drop to determine exflagellation. Exflagellation was measured by adding one drop of room temperature exflaggelation medium to the blood, covering with a cover slip and viewing under a light microscope at 40×.

Mosquitoes infected with P. berghei were maintained at 19-21° C. in a humidified incubator on a twelve-hour day-night cycle and fed on Fructose/PABA solution. At ten to twelve days post-feed mosquito midguts can be dissected to determine the oocyst number. At 21 days post-feed mosquito salivary glands were dissected to obtain infectious sporozoites (21 days is the peak time-point for sporozoite viability, however infectious sporozoites can be obtained from 18 to 28 days post-feed).

1.6.4 Cryopreservation of pRBCs

To allow continued use of the same parasite strain, stocks of pRBCs were cryopreserved. Mosquitoes were fed on parasite-infected mice and the salivary glands were dissected 21 days post-feed using a dissecting microscope and two 1 ml insulin syringes. The salivary glands were gently dissociated using a tissue homogenizer with RPMI-1640 to release the sporozoites. The sporozoites were then counted using a haemocytometer and diluted to 10 000 sporozoites/ml in RPMI-1640. To infect mice, 1000 sporozoites (100 μl ) were injected i.v. into the lateral tail vein. Mice were monitored from six days after injection via thin blood films and once parasitaemia was between 5-10% mice were cardiac bled with 300 U/ml heparin to prevent clotting. The blood was then mixed with an equal volume of P. berghei freezing medium containing 20% DMSO, aliquoted into vials which were subsequently snap-frozen in liquid phase liquid nitrogen (LN2). Stocks were stored in vapour phase LN2.

1.6.5 Sporozoite Challenge

To test the efficacy of liver-stage vaccines, vaccinated and naïve control mice were infected with 1000 sporozoites i.v. into the lateral tail vein. Mice were monitored from four or five days post-injection, dependent on mouse and parasite strain, via thin blood films. Once parasite positive blood films had been confirmed on three consecutive days, mice were sacrificed via cervical dislocation. The parasitaemia levels from three blood smears also allowed the calculation of the time to 0.5 or 1% parasitaemia via linear regression, dependent on the spread of data collected. If thin blood films were negative fourteen days post-infection mice were classed as ‘protected’ and were sacrificed by cervical dislocation.

1.6.6 In vivo Imaging Using the IVIS System

In certain challenge experiments, in vivo imaging of mice was also performed using the IVIS 200 imaging system as previously described [22]. When the transgenic parasites contained the luciferase reporter gene, mice were imaged 44 hours post-infection to assay the level of liver-stage burden via bioluminescence of the parasites. Mice were firstly shaved over the area of the liver, then anaesthetised (3.5% isoflurane, 2 L/minute oxygen) and injected with 50 μl 50mg/ml D-luciferin substrate s.c. into the scruff of the neck. Eight minutes after the injection of luciferin, mice were imaged for two minutes with the following settings: binning medium, F/stop 1, excitation filter blocked and emission filter open. Quantification of the bioluminescence signal was performed using the Living Image 4.2 image analysis software program. A region of interest was created around the area of the liver and kept constant for all animals. The measurements were expressed as the total flux of photons emitted per second of exposure time.

1.6.7 Immunofluorescence Antibody Test (IFAT)

P. falciparum 3D7 sporozoites were isolated from the salivary glands of infected mosquitoes; dissection was performed in PBS containing azide to kill the sporozoites. Sporozoites were counted and diluted to 2×10⁵ sporozoites/ml with 100 μl added to each well in an 8-well microscope slide. Slides were then air dried, wrapped in foil and stored in a sealed bag with desiccant at −20° C. until further use. For the IFAT, all steps were performed in the dark at room temperature. Wells were initially blocked for two hours with 1% BSA in PBS/T, washed three times with PBS then serum samples were added at a dilution of 1/100 in PBS. Slides and sera were incubated together for one hour, washed three times followed by the addition of 1/200 Alexa Fluor® 488 conjugated goat anti-mouse IgG secondary antibody in 1% BSA PBS/T. Slides were incubated for 30 minutes, washed three times then mounted with Mowiol and a coverslip. Slides were dried at room temperature overnight in the dark.

1.6.8 Murine in vitro T Cell Killing Assay

1.6.8.1 Preparation of Hepatoma Cells

On Day −1 of the assay, the liver cell line Hepal-6 was plated at 5×10⁴ cells per well in a 96-well flat bottom plate. Prior to plating, the liver cells were labelled with the membrane dye Vybrant® DiD by incubating a suspension of cells (concentration 5×10⁶ cells/ml in Hepal-6 medium) with 10 μl DiD per ml of cells for ten minutes at 37° C. Cells were subsequently washed twice in 15 ml medium by centrifugation at 600×g for three minutes. Cells were counted using a haemocytometer, diluted to 5×10⁵ cells/ml in Hepal-6 medium and 100 μl added per well of the 96-well flat bottom plate. The liver cells were left to form a monolayer overnight at 37° C. 5% CO₂ in a humidified incubator.

1.6.8.2 Infection of Hepatoma Cells with Murine Parasites

On Day 0, P. berghei GFP sporozoites were dissected from the salivary glands of infected female A. stephensi mosquitoes. Sporozoites were counted using a haemocytometer and diluted to 4×10⁵ sporozoites/ml in Hepal-6 medium. Medium was removed from the Hepal-6 liver cells previously prepared, and 40 000 sporozoites were added in 100 μl Hepal-6 medium per well. Plates were then spun at 1600 rpm for five minutes and subsequently incubated at 37° C. 5% CO₂ in a humidified incubator for a minimum of three hours to allow the sporozoites to invade the hepatocytes. To confirm only live sporozoites expressed GFP, sporozoites were heat-killed for twenty minutes at 95° C. prior to addition in the assay.

1.6.8.3 Addition of Cytokines, Drugs or Splenocytes to the Infected Hepatoma Cells

Following the three-hour incubation, the medium was changed to reduce the chance of infection or experimental wells were initiated with the addition of cytokines, drugs or splenocytes. Experimental wells were performed in duplicate, or triplicate where possible. When splenocytes were added, mice were sacrificed and spleens harvested. Enrichment of CD8⁺ cells was performed. To inhibit the action of perforin-mediated cytotoxicity, enriched CD8⁺ splenocytes were pre-incubated with 10 nM concanamycin A for twenty minutes at 37° C. To inhibit the action of cytokines such as IFNγ or TNFα, enriched CD8⁺ splenocytes were resuspended in medium containing blocking antibodies at various concentrations. The percentage of antigen-specific cells was calculated by setting up ICS in parallel to the killing assay.

1.6.8.4 Assessment of Infectivity by Flow Cytometry 24 hours after the addition of splenocytes, cytokines, drugs or fresh medium, cells were removed from plates by incubation for four minutes with 100 μl trypsin. Cells were collected in 400 μl 10% FCS in PBS in cluster tubes, then centrifuged at 2000 rpm for three minutes. Cells were resuspended in 80 μl 2% FCS in PBS. Immediately prior to running the cells on the flow cytometer (LSRII) 5 μl of 1/1000 DAPI was added to stain dead cells. Infectivity was determined by the calculation of GFP⁺ liver cells.

Percentage inhibition was calculated using the following formula:

% Inhibition=1−(test well/average of control wells)×100

### REFERENCES

1. Bendtsen, J. D., et al., Improved prediction of signal peptides: SignalP 3.0. J Mol Biol, 2004. 340(4): p. 783-95.
2. Ny, T., F. Elgh, and B. Lund, The structure of the human tissue-type plasminogen activator gene: correlation of intron and exon structures to functional and structural domains. Proc Natl Acad Sci USA, 1984. 81(17): p. 5355-9.
3. Kozak, M., At least six nucleotides preceding the AUG initiator codon enhance translation in mammalian cells. J Mol Biol, 1987. 196(4): p. 947-50.
4. Yuen, L. and B. Moss, Oligonucleotide sequence signaling transcriptional termination of vaccinia virus early genes. Proc Natl Acad Sci USA, 1987. 84(18): p. 6417-21.
5. Cottingham, M. G., et al., Preventing spontaneous genetic rearrangements in the transgene cassettes of adenovirus vectors. Biotechnol Bioeng, 2012. 109(3): p. 719-28.
6. Gilbert, S. C., et al., A protein particle vaccine containing multiple malaria epitopes. Nat Biotechnol, 1997. 15(12): p. 1280-4.
7. McConkey, S. J., et al., Enhanced T-cell immunogenicity of plasmid DNA vaccines boosted by recombinant modified vaccinia virus Ankara in humans. Nat Med, 2003. 9(6): p. 729-35.
8. Robson, K. J., et al., A highly conserved amino-acid sequence in thrombospondin, properdin and in proteins from sporozoites and blood stages of a human malaria parasite. Nature, 1988. 335(6185): p. 79-82.
9. Berthoud, T. K., et al., Potent CD8+ T-cell immunogenicity in humans of a novel heterosubtypic influenza A vaccine, MVA-NP+Ml. Clin Infect Dis, 2011. 52(1): p. 1-7.

10. Pascolo, S., et al., HLA-A2.1-restricted education and cytolytic activity of CD8(+) T lymphocytes from beta2 microglobulin (beta2m) HLA-A2.1 monochain transgenic H-2Db beta2m double knockout mice. J Exp Med, 1997. 185(12): p. 2043-51.
11. Rozen, S. and H. Skaletsky, Primer3 on the WWW for general users and for biologist programmers. Methods Mol Biol, 2000. 132: p. 365-86.
12. Bednarek, M. A., et al., The minimum peptide epitope from the influenza virus matrix protein. Extra and intracellular loading of HLA-A2. J Immunol, 1991. 147(12): p. 4047-53.
13. Gotch, F., et al., Cytotoxic T lymphocytes recognize a fragment of influenza virus matrix protein in association with HLA-A2. Nature, 1987. 326(6116): p. 881-2.
14. Li, W. C., K. L. Ralphs, and D. Tosh, Isolation and culture of adult mouse hepatocytes. Methods Mol Biol, 2010. 633: p. 185-96.
15. Kim, Y., et al., Immune epitope database analysis resource. Nucleic Acids Res, 2012. 40(Web Server issue): p. W525-30.
16. Rammensee, H., et al., SYFPEITHI: database for MHC ligands and peptide motifs. Immunogenetics, 1999. 50(3-4): p. 213-9.
17. Roederer, M., J. L. Nozzi, and M. C. Nason, SPICE: exploration and analysis of post-icytometric complex multivariate datasets. Cytometry A, 2011. 79(2): p. 167-74.
18. Burbelo, P. D., R. Goldman, and T. L. Mattson, A simplified immunoprecipitation method for quantitatively measuring antibody responses in clinical sera samples by using mammalian produced Renilla luciferase-antigen fusion proteins. BMC Biotechnol, 2005. 5: p. 22.
19. Burbelo, P. D., et al., Antibody profiling by Luciferase Immunoprecipitation Systems (LIPS). J Vis Exp, 2009(32).

20. Franke-Fayard, B., et al., A Plasmodium berghei reference line that constitutively expresses GFP at a high level throughout the complete life cycle. Mol Biochem Parasitol, 2004. 137(1): p. 23-33.

21. Lin, J. W., et al., A novel ‘gene insertion/marker out’ (GIMO) method for transgene expression and gene complementation in rodent malaria parasites. PLoS One, 2011. 6(12): p. e29289.
22. Ploemen, I. H., et al., Visualisation and quantitative analysis of the rodent malaria liver stage by real time imaging. PLoS One, 2009. 4(11): p. e7881.
23. Guguen-Guillouzo, C., et al., High yield preparation of isolated human adult hepatocytes by enzymatic perfusion of the liver. Cell Biol Int Rep, 1982. 6(6): p. 625-8.
24. Silvie, O., et al., A role for apical membrane antigen 1 during invasion of hepatocytes by Plasmodium falciparum sporozoites. J Biol Chem, 2004. 279(10): p. 9490-6.

25. Renia, L., et al., A malaria heat-shock-like determinant expressed on the infected hepatocyte surface is the target of antibody-dependent cell-mediated cytotoxic mechanisms by nonparenchymal liver cells. Eur J Immunol, 1990. 20(7): p. 1445-9.

### 2 ASSESSING IMMUNOGENICITY AND EFFICACY OF EIGHT CANDIDATE P. FALCIPARUM VACCINES IN MICE

2.1 Introduction

Eight new pre-erythrocytic malaria vaccines were developed based on eight candidate liver-stage antigens. The aim was to comparatively screen both immunogenicity and efficacy of these vaccines in mice.

The liver-stage of malaria is known to be the target of CD8⁺ T cells, possibly through IFNγ. For this reason, the ex vivo IFNγ ELISpot has been used as the assay of choice to measure cellular immunogenicity in vaccine trials. However, most vaccine trials have failed to identify consistent correlates of protection [1, 4, 12, 29-31]. A multitude of factors could be involved and analysed, including alternative cytokine responses other than IFNγ, memory responses, chemokines and chemokine receptors as well as T cell trafficking to various organs. Assessing all such factors at once in vitro would require an immense amount of reagents and time, but multi-parameter flow cytometry offers the opportunity to look at multiple cytokines and markers from both CD8⁺ and CD4⁺ T cells. For the pre-erythrocytic antigen screen undertaken in this section, in addition to IFNγ, the cytokines TNFα and IL-2 were also assessed as well as the degranulation marker CD107a; this constitutes the standard panel of markers recommended for assessment [32]. CD107a-expressing CD8⁻ T cells represent cells capable of cytotoxic killing in an antigen-specific manner [37], which may have a role in protection against liver-stage malaria. As the majority of the candidate antigens are also expressed at either the sporozoite or blood-stage (apart from PfLSA1 and PfLSAP1), and these stages are known targets of antibody-mediated immunity [30, 38], relative antibody levels were also assessed.

Whilst it is important to determine that vaccines induce an immune response, the measures listed above do not necessarily indicate functional immunogenicity. That is, whether the T cells (or antibodies) that are induced by vaccination have the capability of inhibiting liver-stage malaria parasites (or other forms). It has historically been extremely difficult to assess functionality of immune responses directed at the P. falciparum liver-stage, for a number of reasons. First, P. falciparum cannot infect commonly used small animal models such as mice, and whilst non-human primates including Aotus monkeys can be infected with human malaria parasites they are not widely available and cost is a limiting factor. Species of malaria that infect rodents are commonly used to study the liver-stage of infection, such as P. berghei and P. yoelii, yet it is not clear how well studies using these models reflect P. falciparum infections in humans. Furthermore, many of the newly identified P. falciparum antigens do not have known rodent malaria homologs, making such studies currently impossible for those antigens. Second, unlike blood-stage vaccines where functional antibody responses can be tested in vitro using the growth inhibition assay, no such assay currently exists for the liver-stage of malaria.

A new model that has been increasingly used is the generation of transgenic P. berghei parasites that express a particular P. falciparum (or P. vivax) gene. Two methods have commonly been used, either replacement of the endogenous P. berghei gene with the P. falciparum homolog under control of the relevant P. berghei promoter, or addition of the P. falciparum copy of the gene inserted at a different and dispensable point in the genome. Such transgenic parasites allow assessment of efficacy of P. falciparum or P. vivax sub-unit vaccines in mice, using P. berghei expressing the appropriate human malaria antigen as the challenge agent. This strategy was used to develop ten transgenic P. berghei parasites expressing each of the eight candidate antigens studied in this study, together with CSP and TRAP as controls. The addition strategy was employed; the P. falciparum antigens were placed under control of the P. berghei UIS4 promoter, given not all the candidates have P. berghei homologs, and inserted at the P. berghei 230p locus. P. berghei UIS4 is expressed at both the sporozoite and liver-stage, and hence antigens placed under control of this promoter will also be expressed at these stages regardless of their native expression profile. This allows the immune response to each antigen to be comparatively screened, given all the targets will have the same expression level and profile. For antigens with known P. berghei homologs, PfCe1TOS, PFI0580c and PfUIS3, efficacy was initially assessed with a P. berghei wild-type challenge.

2.2 Results

2.2.1 All Vaccines Elicit a Cellular Immune Response as Measured by ex vivo IFNγ ELISpot

The viral vectored vaccines were assessed for their relative levels of cellular immunogenicity by ex vivo spleen IFNγ ELISpot. The vaccines were delivered in an eight-week interval ChAd63 prime MVA boost regimen, and the cellular immune response was measured at two weeks post-boost and compared to data collected at two weeks post-prime section 2 (representing peak time-points post immunization [47, 48]). Immunogenicity was measured in two different strains of mice with different immune profiles: Balb/c, which preferentially produce Th2 cytokines, and C57BL/6, which preferentially produce Th1 cytokines [49-52]. Each vaccine induced a measurable immune response in Balb/c mice (FIG. 2). The MVA boost was able to return the IFNγ response to at least the level seen after the priming vaccination. For both PfUIS3 and PfLSA1, the boost vaccination significantly increased the IFNγ response above that observed two weeks after the prime, p<0.0001. The antigens are listed on the x-axis in increasing size order, and as can be seen, there was no clear trend between antigen size and the magnitude of the IFNγ response.

In C57BL/6 mice, no detectable response was observed after vaccination with either PfLSAP1 or PfLSA1. MVA-PFE1590w was unable to boost the IFNγ response to the level observed two weeks after the prime. For the remaining antigens, the MVA vaccination was able to boost the response to at least the level observed two weeks after the prime, with PfCe1TOS, PfUIS3 and PfLSA3 showing an increase in the median response post-boost compared to post-prime. The overall magnitude of the IFNγ responses was greater in C57BL6 as compared to Balb/c (FIG. 2A compared to B), as was the variation between mice. As all vaccines induced a cellular response in Balb/c mice, this strain was chosen to compare the efficacy of the eight vaccines against malaria challenge.

2.2.2 The Cellular Response to the Eight P. falciparum Vaccines is Predominantly CD8⁺ T Cell-Mediated

ICS was performed to determine whether the response was mediated predominantly by CD8⁺ or CD4⁺ T cells and whether other cytokines were also secreted in response to ex vivo antigen stimulation. Two time points were assessed in the blood, corresponding to the peak of the response after adenovirus (two weeks post-prime) or MVA vaccination (one week post-boost), in addition to two weeks post-boost in the spleen. In addition to IFNγ, cells were stained for production of TNF_60 and IL-2, and the cell surface localisation of the degranulation marker CD107a. The responses two-weeks post-prime were just above the limit of detection and as such the data is not shown. For all vaccines in both the blood and the spleen post-boost, in both strains of mice, it was found that the response was predominantly CD8⁺ T cells producing IFNγ or TNFα or expressing CD107a, with minimal levels of IL-2-secretion, as detailed below.

In Balb/c mice, the cytokine profiles were quite different in the blood compared to the spleen. In the blood the greatest CD8⁺ IFNγ⁺ responses were to antigens PfLSA1, PfLSA3 and PfUIS3 (medians of 4.2%, 4.8% and 6.3% respectively) (FIG. 3). PfLSA1 and PfLSA3 also showed the highest CD8⁺ TNFα⁺ responses (medians of 7.3% and 6.4% respectively) and CD8⁺ CD107a⁺ responses (medians of 17.1% and 12.1% respectively). The CD4⁺ responses were much lower than the CD8⁺ responses for all antigens, at less than 1% for each cytokine. In summary, in Balb/c mice in the blood one week post-boost, the response was predominantly CD8⁺ T cells producing IFNγ, TNFα or expressing CD107a with the highest magnitude for PfLSA1 and PfLSA3.

In the spleen, the highest cytokine responses were observed for the antigens PfUIS3 and PfLSA1 (FIG. 4). This was the case for CD8⁺ IFNγ⁺ (14% and 5.8% respectively), CD8⁺ TNFα⁺ (11.4% and 5.3% respectively) and CD8⁺ CD107a⁺ (13.9% and 6.6%). The CD4⁺ response was slightly higher than that seen one-week earlier in the blood, with the majority of responses less than 2%. The highest CD4⁻ response was CD4⁺ IFγ⁺ cells with a median of 1.4% for PfLSA3. There was also some detectable CD4⁺ IL-2⁺, approximately 0.5% for most antigens, with the response trending towards being dependent on antigen size (antigens are listed in order of increasing size on the x-axis). In summary, in Balb/c mice in the spleen two weeks post-boost, the response was predominantly CD8⁺ T cells producing IFNγ, TNFα or expressing CD107a with the highest magnitude for PfUIS3 and PfLSA1 in each case. Considering both the responses in the blood and the spleen, the antigens PfUIS3, PfLSA1 and PfLSA3 were the most immunogenic in Balb/c mice.

In C57BL/6 mice, the cytokine profile was similar between the blood and spleen post-boost, and hence results are shown for the spleen only. The cytokine staining confirmed the results obtained by ELISpot that there were no T cell epitopes for the antigens PfLSAP1 and PfLSA1 in C57BL/6 mice (FIG. 5). Compared to Balb/c mice, the median responses were generally higher in C57BL/6 mice and a greater number of antigens had strong responses, consistent with data obtained by IFN-γ ELISpot. PfUIS3, PfLSA3, PfCe1TOS and PFI0580c demonstrated the highest CD8⁺ response measured by IFNγ⁺, TNα⁺ or CD107a⁺. The IL-2⁺ responses for both CD8⁺ and CD4⁺ were low, as were the CD4⁺ responses in general (less than 2%), but in each case the pattern of antigens responding with the highest magnitude was essentially the same. In summary, for C57BL/6 mice in both the blood and the spleen, the response was predominantly CD8⁺ T cells producing IFNγ, TNFα or expressing CD107a with the highest magnitude for the antigens PfUIS3, PfLSA1, PfLSA3 and PFI0580c.

2.2.3 Vaccination with the Pre-Erythrocytic Candidate Antigens Can Also Induce an Antibody Response

Given most antigens are expressed at either the sporozoite or the blood stage, it is plausible that vaccination with these antigens could provide some degree of protective efficacy through an antibody-mediated effect. Therefore antibody levels in serum samples were measured using the LIPS assay. In brief, this system allows the measurement of antibody when protein samples are not available to perform standard ELISAs. Instead, genetic constructs are designed that fuse the antigen of interest to the Renilla luciferase reporter gene. The expressed construct can then be used to measure antibody in the LIPS assay, with luminescence (light units) as the read-out. Constructs were designed, cloned and transfected for each of the eight candidate antigens.

Antibody levels were assessed at both five to six weights post-prime (D35-42) and two weeks post-boost (D70). In Balb/c mice, only vaccination with the antigens PfUIS3, PfLSAP2 and PfI0580c generated a detectable antibody response after the ChAd63 prime vaccination (FIG. 6). No antibody responses above background levels were detected against PfLSAP1 at any time-point measured. All other vaccines generated a detectable antibody response after the MVA boost; this represented a small increase from the antibody level at D35-42 for PfLSA3 (p=0.028) and a significant increase for all other antigens (p=0.01-0.001).

The pattern of antibody responses observed in C57BL/6 was different to that observed in Balb/c mice. No antibody responses were detected at any time-point for PfLSAP1, PFE1590w, PfLSAP2 and PfLSA1 (FIG. 7). After the prime vaccination, antibody responses were only detected to PFI0580c. However after the boost vaccination, antibody responses were detectable against PfCe1TOS, PfUIS3 and PfLSA3, in addition to PFI0580c. This represented a significant increase from the antibody levels at D42 for both PfUIS3 (p=0.0112) and PfLSA3 (p=0.0286). Whilst there was a trend towards an increase at D70 for PfCe1TOS and PFI0580c, this was not significant. Interestingly, PFI0580c generated one of the highest relative antibody levels in C57BL/6 mice; this level was reached by D42 and did not increase significantly after the MVA boost.

As each antigen measured in the LIPS assay generated a different background response, analysis was then performed to measure the fold change from the naïve (background) antibody level to the level reached after the boost vaccination (D70). This enabled the antigens to be compared side-by-side (FIG. 8). In Balb/c mice, vaccination with PFI0580c and PfLSAP2 generated the highest levels of antibodies (fold change of 1.6 and 1.5 respectively). The remaining antigens were all comparable, with a fold change from background of 1.3 (excluding PfLSAP1). In C57BL/6 mice, vaccination with PFI0580c resulted in the highest level of antibody production, with a fold change from background of 1.5. The next highest levels were seen for PfLSA3 (1.3), PfCe1TOS and PfUIS3 (both 1.2). In summary, vaccination with PFI0580c generated the highest antibody response in both strains of mice, with the relative antibody levels comparable between C57BL/6 and Balb/c mice.

2.2.4 Vaccination with PfIUIS3 Results in a Delay in Time to 1% Parasitaemia Upon Heterologous Challenge with P. berghei Wild-Type Sporozoites

After measuring the relative immunogenicity elicited by each candidate antigen in a prime-boost vaccination regimen, the ability of these vaccines to protect against malaria was then assessed. Since P. falciparum does not infect mice, other challenge models were investigated. According to PlasmoDB, P. berghei homologs only exist for the P. falciparum candidate antigens PfCe1TOS, PFI0580c and PfUIS3. A relatively high sequence similarity exists between the

P. falciparum and P. berghei protein sequences; 65% similarity for Ce1TOS, 54% for UIS3 and 52% for PFI0580c, calculated using the European Bioinformatics Institute EMBOSS needle pair-wise protein sequence alignment tool (http://www.ebi.ac.uk/Tools/psa/emboss_needle/). For this reason, it was assumed that if any epitopes fell in the regions of similarity it may be possible to see cross species protection after vaccination with the P. falciparum antigen and challenge with P. berghei sporozoites.

Protective efficacy of ChAd63-MVA PfCe1TOS vaccination was assessed in both Balb/c and C57BL/6 mice, given previous studies had demonstrated cross-species protection with PfCe1TOS protein vaccination [55, 56]. Mice were vaccinated with the standard eight-week prime boost regimen, with blood collected six days after MVA boost to assess cellular immunogenicity via ICS prior to challenge two days later (eight days post-boost) with 1000 P. berghei wild-type sporozoites injected intravenously. Despite a good cellular immune response observed in C57BL/6 mice (median 8.8% CD8⁺ cells secreting IFNγ), vaccination with ChAd63-MVA PfCe1TOS failed to protect against challenge with P. berghei sporozoites. Antibodies were not measured in this experiment, but previous data indicated that post-boost PfCe1TOS vaccination results in a comparatively strong antigen-specific antibody response in C57BL/6 mice (FIG. 7). Given PfCe1TOS vaccination in Balb/c mice resulted in low level cellular immunogenicity in the blood as measured by ICS (negligible cytokine secretion, median 5.9% CD8⁺ cells expressing CD107a), it was not surprising that such vaccination did not result in any protective efficacy against P. berghei challenge. Antibodies were not assessed in this experiment but previous data indicated there was little to no antigen-specific antibody production after PfCe1TOS vaccination in Balb/c mice. This experiment was repeated (in Balb/c mice only) and the same result was found.

Protective efficacy against heterologous P. berghei challenge was assessed for PFI0580c vaccination in Balb/c mice, again following the standard eight-week interval prime-boost regimen. Cellular immunogenicity was assessed in the blood six days post MVA boost by ICS, and was very low (less than 0.5% for each cytokine from CD8⁺ T cells). As for PfCe1TOS, data is only shown for CD8⁺ T cells producing IFNγ or TNFα, or expressing the degranulation marker CD107a. Antibodies were not measured in this experiment but previous data indicated that PFI0580c vaccination in Balb/c mice resulted in high levels of antigen-specific antibodies, greater than vaccination with any other antigen. However, no protection was seen upon challenge with 1000 P. berghei sporozoites eight days post MVA boost.

Protective efficacy against heterologous P. berghei challenge was assessed for PfUIS3 vaccination in Balb/c mice. ICS analysis demonstrated a moderate immune response, with medians of 1.9% IFNγ⁺, 3% TNFα⁺ and 4.8% CD107a⁺ from CD8⁺ T cells (FIG. 9A). This was comparable to the results previously found in the blood post MVA boost for PfUIS3 vaccination in Balb/c mice (FIG. 3), although the median CD8⁺ IFNγ⁺ response was slightly lower. Data is not shown for CD4⁺ T cells or IL-2⁺ due to low-level responses, and again antibodies were not measured in this experiment but previous data showed positive antigen-specific antibody responses to PfUIS3 in Balb/c mice, with relatively high levels compared to those induced by the other antigens (FIG. 8). Upon i.v. challenge with 1000 P. berghei sporozoites eight days post MVA boost, there was a significant delay in the time taken for parasitaemia in the blood to reach 1% in vaccinated mice compared to controls, p=0.0048 Log-rank (Mantel-Cox) Test (FIG. 9 B). The delay in time to 1% parasitaemia was calculated by linear regression of the parasitaemia collected over three consecutive days. No mice were sterilely protected.

In summary, protective efficacy against heterologous P. berghei wild-type challenge was assessed after vaccination with the P. falciparum antigens for which there are P. berghei homologs, PfCe1TOS, PFI0580c and PfUIS3. These P. falciparum antigens have relatively high protein sequence similarity with their P. berghei homologs, of over 50%. Protective efficacy was assessed for each antigen in Balb/c mice, and additionally in C57BL/6 mice for PfCe1TOS. No protection was seen after ChAd63-MVA vaccination with PfCe1TOS or PFI0580c. There was a significant delay in time to 1% parasitaemia after vaccination with ChAd63-MVA PfUIS3 and challenge with P. berghei sporozoites.

2.2.5 Assessment of Protective Efficacy of the Eight P. falciparum Candidate Vaccines Using Transgenic P. berghei Sporozoites Expressing the Cognate P. falciparum Antigen

Since P. falciparum does not infect mice, an alternative challenge model was required to assess the efficacy of the new viral vectored vaccines. As only three antigens contained P. berghei homologs, transgenic P. berghei parasites were developed that expressed the relevant P. falciparum antigen (in addition to the P. berghei copy of that antigen, if it existed). Each P. falciparum antigen was expressed under control of the P. berghei UIS4 promoter using the additional strategy (insertion at the 230p locus). To allow in vivo assessment of liver-stage infection, each transgenic parasite also contained the luciferase gene.

As fitness assessments of these transgenic parasites had not been undertaken, a standard challenge dose of 1000 sporozoites per mouse injected i.v. was used with all experiments. Experiments were performed in Balb/c mice to allow comparison between antigens (as not all vaccines were immunogenic in C57BL/6 mice). Furthermore, C57BL/6 mice succumb more quickly to P. berghei infection than Balb/c mice [57, 58]; using Balb/c mice therefore allowed greater discrimination of small differences of protectiveness between candidate antigens. Mice were vaccinated in the standard eight-week interval prime-boost regimen, with blood collected six days post MVA boost to check immunogenicity before proceeding with the challenge. This data is not shown but was comparable to that seen in the immunogenicity studies. For each transgenic parasite line, vaccinated mice were challenged eight days post MVA boost together with eight unvaccinated controls. The prime-boost regimen was varied slightly for PFI0580c, PFE1590w and PfLSAP2; due to failed sporozoite production mice were given a second MVA boost four weeks after the original boost, and challenged eight days after the second boost. Each transgenic parasite line resulted in different blood parasitaemia kinetics, and hence each vaccination-challenge experiment is presented on a separate survival graph (FIG. 10). No protection was conferred by vaccination with PfLSAP1, PFE1590w, PfCe1TOS and PfLSA3, with PfCe1TOS vaccination resulting in a significantly shorter time to 1% parasitaemia than seen in the control mice (p=0.0291, Log-rank (Mantel-Cox) Test), suggesting a negative effect of the vaccination. The transgenic parasite P. berghei PfLSA3 did not efficiently infect all eight naïve controls; three mice showed no signs of parasites in the blood at fourteen days post challenge.

Four vaccination regimens resulted in a significant level of protection when comparing vaccinated mice with controls by the Log-rank (Mantel-Cox) Test: PFI0580c (p=0.0072), PfUIS3 (p=0.0001), PfLSAP2 and PfLSA1 (both p<0.0001), as a result of sterile protection or a delay in time to 1% parasitaemia (FIG. 10). Mice were classified as sterilely protected when there was no evidence of parasites in the blood up to and including the experiment end-point, fourteen days post-challenge. PfLSA1 and PfLSAP2 conferred sterile protection in seven out of eight vaccinated mice (87.5%) (Table 2.1), whilst only one PfUIS3 vaccinated mouse was sterilely protected (12.5%). The identical vaccination-challenge experiments were also performed for the antigens CSP and TRAP (survival curves not shown), with CSP resulting in 25% sterile protection and TRAP resulting in no sterile protection (Table 2.1).

TABLE 2.1
Sterile protection from transgenic P. berghei sporozoite
challenge after vaccination with the eight P. falciparum
candidate antigens: comparison with PfCSP or PfTRAP
vaccination. Analysis of sterile protection in mice
challenged in Figure. Mice remaining slide negative
until fourteen days post-challenge were considered
sterilely protected. The eight new candidate
antigens are listed in increasing size order.

| Vaccine | Sterile Protection (%) |
|---|---|
| PfCSP | 25* |
| PfTRAP | 0 |
| PfLSAP1 | 0 |
| PFE1590w | 0 |
| PfCelTOS | 0 |
| PfUIS3 | 12.5 |
| PfLSAP2 | 87.5 |
| PFI0580c | 0 |
| PfLSA1 | 87.5 |
| PfLSA3 | 25** |

*Challenge of naïve mice with transgenic P. berghei expressing P. falciparum CSP resulted in only seven out of eight mice becoming infected with malaria.
**Challenge of naïve mice with transgenic P. berghei expressing P. falciparum LSA3 resulted in only five out of eight mice becoming infected with malaria.

In order to compare the delay in time to 1% parasitaemia (tt1%) across vaccines and transgenic parasite strains, the median delay was calculated by the following formula: (tt1% of vaccinee)−(average tt1% of controls). This then accounts for the various fitness levels of the transgenic parasites (the differing blood parasitaemia kinetics). Mice that were sterilely protected, or not infected, were not included in this analysis. Vaccination with PfCSP or PfUIS3 resulted in a significant delay in time to 1% parasitaemia (p=0.004), as did PFI0580c (p=0.0072), whilst no delay was observed after vaccination with PfTRAP, PfLSAP1, PFE1590w, PfCe1TOS and PfLSA3 (FIG. 11). Vaccines are listed in increasing size order on the x-axis, after the control vaccines CSP and TRAP. Only one PfLSA1 or PfLSAP2 vaccinated mouse became parasitaemic, so whilst statistical analysis cannot be performed, FIG. 11 indicates that those mice did have a greater median delay than the naïve controls or mice vaccinated with antigens that resulted in no protection.

A summary of immunogenicity and efficacy for each candidate antigen is provided in Table 2.2. The level of cellular (ELISpot and ICS) or humoral (LIPS) immunogenicity of the candidate antigens did not necessarily predict a delay in parasitaemia or sterile protection. PfLSAP1 resulted in low levels of cellular immunogenicity (+) and no humoral immunogenicity (−), and therefore the absence of any protective efficacy was not surprising. However, PFE1590w and PfCe1TOS both resulted in moderate levels (++) of cellular immunogenicity, yet no protection was seen. PfLSA3 resulted in high levels of cellular immunogenicity (+++) and reasonable levels of humoral immunogenicity (++) and still no protection was observed. Of the antigens that did provide protection, PfUIS3 and PFI0580c exhibited reasonable to high levels of both cellular and humoral immunogenicity and resulted in a delay in time to 1% parasitaemia, yet the immune responses to these antigens were comparable to the levels of both PfLSA1 and PfLSAP2 vaccination, which resulted in sterile protection. In each transgenic challenge experiment mice were assessed for liver-stage parasite burden at 44 hours post challenge by in vivo imaging (as the parasites expressed luciferase). For all vaccines that provided protection as determined by blood parasitaemia, protection was also evident by in vivo imaging of luciferase.

TABLE 2.2
Summary of the cellular and humoral immunogenicity and protective
efficacy of the eight candidate P. falciparum antigens in Balb/c mice.
Cellular immunogenicity is based on both ex vivo IFNγ ELISpot
responses and ICS and antigens are ranked against each other. Humoral
immunogenicity is based on antibody measurements via the LIPS assay.
Protective efficacy is given after challenge with transgenic P. berghei
sporozoites expressing the cognate P. falciparum antigen. Delay refers
to a significant delay in the time to 1% parasitaemia compared to naïve
control mice. PfLSA3 was classed as having 0% sterile protection given
more naïve mice than vaccinated mice were not infected with malaria.

| | Cellular | Humoral | |
|---|---|---|---|
| Vaccine | Immunogenicity | Immunogenicity | Protection |
| PfLSAP1 | + | − | 0 |
| PFE1590w | ++ | ++ | 0 |
| PfCelTOS | ++ | + | 0 |
| PfUIS3 | +++ | ++ | 12.5%, Delay |
| PfLSAP2 | ++ | +++ | 87.5% |
| PFI0580c | ++ | +++ | Delay |
| PfLSA1 | +++ | ++ | 87.5% |
| PfLSA3 | +++ | ++ | 0 |

2.3 Discussion

This confirmed that all the viral vectored vaccines were expressing their target antigens, and induced high levels of IFNγ in a prime-boost regimen as measured by spleen ELISpot. The responses induced were of greater magnitude than immunization with the target antigens in different vaccine platforms, confirming that viral vectors are excellent inducers of cellular immunogenicity. Approximately twice the response was observed after ChAd63-MVA vaccination than previously observed by vaccination with either PfCe1TOS protein [55], PfLSA1 protein [59] or PfLSA3 DNA [60]. The only regimen with comparable immunogenicity was a recent paper using PfLSA1 and PfCe1TOS DNA vaccination (3×30 μg) with in vivo electroporation [61]. Surprisingly, the boosting effect of the MVA only returned the IFNγ level measured by ELISpot to that seen after the prime, for most antigens. However, a clear difference was seen by ICS, as no detectable responses were measured post-prime but were measurable post-boost. As this study encompassed the greatest number of pre-erythrocytic antigens so far tested in the ChAd63-MVA prime-boost regimen, the ELISpot results may reflect variability or different kinetics leading to the peak time-point in the spleen being missed. Overall, PfUIS3, PfLSA1, PfCe1TOS and PfLSA3 vaccination, dependent on mouse strain, induced the greatest IFNγ responses.

The IFNγ response measured was induced predominantly through CD8⁺ T cells, with minimal CD4⁺ responses, confirming that viral vectors are excellent at inducing CD8⁺ T cells. Most cells also produced TNFaα and expressed CD107a, suggesting the cells were capable of cytotoxic activity. As the spleen is a secondary lymphoid organ and immune cells travel through the blood to perform their effector functions, initial vaccine induced responses were assessed in these organs. For vaccines targeting liver-stage malaria, the effector immune cells must home to the liver in order to kill the intrahepatic parasites, therefore it is of interest to determine whether T cells induced by these viral vectored vaccines home to the liver. Since CD8⁺ T cells induced by the candidate vaccines produced multiple cytokines, it will also be important to determine whether polyfunctional cells contribute to a protective immune response. For the vaccines that induced the highest levels of protective immunity, PfUIS3, PfLSA1 and PfLSAP2, both the polyfunctionality and the ability to home to the liver was assessed in experiments described below.

All vaccines (except PfLSAP1) induced detectable antibody responses post-boost. The highest relative responses were to PFI0580c and PfLSAP2, both antigens that are either expressed at the sporozoite or blood-stage in addition to the liver-stage. Such a finding confirms previous results that the viral vectored platform can induce high antibody titres in addition to cellular responses.

Overall, cellular immune responses were greater in C57BL/6 mice than Balb/c mice, whilst humoral responses were comparable in both strains. This may be due to the greater innate capacity of the C57BL/6 strain to produce IFNγ compared to Balb/c [49-51]. Interestingly, despite the overall cellular immunity observed in C57BL/6 mice, not all antigens were capable of inducing a response (PfLSAP1 and PfLSA1), demonstrating the limited MHC repertoire of outbred mice. To overcome this limitation, multiple strains of mice can be used, or outbred mice. Outbred mice will exhibit greater variability, so are not ideal for initial screening studies, but are more representative of an outbred human population.

Surprisingly, ChAd63-MVA PfCe1TOS did not induce heterologous protection against P. berghei wild-type challenge in Balb/c or C57BL/6 mice, nor homologous protection against transgenic PbPfCe1TOS sporozoites. This was unexpected given adjuvanted PfCe1TOS protein has previously induced cross-species protection (60% sterile) in both Balb/c and outbred mice [55]. The protection observed was likely dependent on antibodies given the cellular response measured by IFNγ ELISpot was only a median of 100 SFC per million splenocytes, much lower than observed in this current study. However, a recent study by the same group utilizing bacteria as a vector demonstrated 60% homologous protection with PbCe1TOS and 55% heterologous protection with PfCe1TOS against P. berghei challenge [69]. This seems surprising given there was a negligible production of antibodies and less than 60 SFC per million splenocytes measured by IFNγ ELISpot. Furthermore, results from our laboratory identified no protection from ChAd63-MVA PbCe1TOS vaccination against homologous P. berghei challenge (Karolis Bauza, DPhil Thesis). One critical difference between the studies of Bergmann-Leitner and our laboratory was the route of sporozoite injection, with Bergmann-Leitner challenging by sub-cutaneous injection versus intravenous injection in this study. Intravenous injection is a more stringent challenge model [70], so perhaps efficacy would be observed in the ChAd63-MVA regimen if sub-cutaneous challenge was used. If the results from the planned PfCe1TOS clinical trial [5] prove successful and efficacy is associated with induced antibodies, a protein boost could be combined with the viral vectored approach to increase the antigen-specific antibody titre generated in this regimen [71, 72].

ChAd63-MVA PfUIS3 vaccination was able to induce protection against both homologous and heterologous challenge, seen as a delay in time to blood-stage parasitaemia. Whilst cross-species protection has been demonstrated in irradiated and genetically attenuated sporozoite models [73-75], this is only the second report utilizing a pre-erythrocytic sub-unit vaccine (the other being PfCe1TOS). The most significant finding was that both PfLSA1 and PfLSAP2 induced 87.5% sterile protection (7/8 mice). This was greater than the protective efficacy induced by ChAd63-MVA TRAP or CSP, and provides an excellent proof-of-concept that better target antigens do exist. PfLSAP2 was only recently identified as a liver-stage antigen [76], and these results mark the first studies of PfLSAP2 as a vaccine candidate. PfLSA1 was identified as a promising target in 1992 when an association was found with PfLSA1, HLA-B53 and resistance to severe malaria in Africa [77]. As there are no murine malaria homologs pre-clinical studies have been limited, yet PfLSA1 has consistently been associated with protection in studies of natural immunity and irradiated sporozoite immunization [78-81], and therefore clinical studies with this candidate should be considered.

Interestingly, vaccination with CSP provided a higher level of protection than with TRAP; this was not necessarily a surprising result, given a head to head comparison found PbCSP was more protective than PbTRAP [82], and in humans protective efficacy following ME-TRAP vaccination requires extremely high levels of T cells [4]. Nevertheless, at least a low level of protection might have been expected with TRAP and this finding highlights how little we truly understand about translating results from murine studies to the clinic. Of the other vaccines tested, PFI0580c also provided a delay in the time to blood-stage parasitaemia, albeit reduced compared to the delay induced by PfUIS3 or CSP. A combination vaccine of the P.yoelii version of PFI0580c and PyUIS3 has shown efficacy in outbred mice and hence the current finding suggests that both antigens should be further investigated. Neither PfLSAP1, PFE1590w nor PfLSA3 provided protection against transgenic challenge. This is the first time PfLSAP1 and PFE590w have been assessed as targets for a malaria vaccine, and hence there were no preconceived ideas on how these antigens may or may not perform. PfLSA3, however, has previously induced protection in mice [60], chimpanzees [83] and monkeys [84], yet the absence of reported data from a clinical trial due for completion in 2008 (ClinicalTrials.gov identified NCT00509158) suggests a lack of efficacy in humans.

Excitingly, both PfLSAP2 and PfLSA1 induced moderate cellular immune responses compared to the levels of CSP required to provide protection in murine models (Pb9 epitope, 20-30% antigen specific CD8⁺ cells) [85, 86], suggesting that such immunogenicity and efficacy could be attainable in a clinical setting. Interestingly, the magnitude of the immune response to the various candidate antigens did not predict which vaccines would be protective, as PfLSA3 vaccination induced strong immunogenicity and yet no efficacy was seen. This supports the notion that not only the magnitude, but also the quality of the immune response is important in eliciting protection. These findings also indicate that the antigenic target is of high importance, and suggests that both PfLSA1 and PfLSAP2 are potentially better targets than CSP or TRAP for a pre-erythrocytic vaccine. The efficacy seen by these vaccines needs to be confirmed and assessed in other strains of mice to ensure it is not H-2^{d} restricted; further assessment of these candidate vaccines was performed and the results are described below.

In summary, these results demonstrated the immunogenicity and protective efficacy of the eight candidate antigens. All antigens were immunogenic when administered in the standard eight-week interval prime-boost regimen, producing predominantly CD8⁺ cells secreting IFNγ and TNFα and expressing CD107a, with most vaccines also inducing detectable levels of antibodies. PfUIS3, PfLSA1, PfLSA3 and PfCe1TOS induced the highest cellular responses, whilst PfLSAP2 and PFI0580c induced the highest antibody responses. PfLSA1, PfLSAP2 and PfUIS3 were capable of inducing greater protective efficacy than demonstrated for PfCSP or TRAP, providing excellent proof-of-concept that better target antigens do exist, as has recently been shown for blood-stage vaccines [87].

### REFERENCES

1. Agnandji, S. T., et al., A phase 3 trial of RTS,S/AS01 malaria vaccine in African infants. N Engl J Med, 2012. 367(24): p. 2284-95.
2. Asante, K. P., et al., Safety and efficacy of the RTS,S/AS01E candidate malaria vaccine given with expanded-programme-on-immunisation vaccines: 19 month follow-up of a randomised, open-label, phase 2 trial. Lancet Infect Dis, 2011. 11(10): p. 741-9.
3. Olotu, A., et al., Four-year efficacy of RTS,S/AS01JE and its interaction with malaria exposure. N Engl J Med, 2013. 368(12): p. 1111-20.
4. Ewer, K. J., Protective CD8+ T cell Immunity to Human Malaria Induced by Chimpanzee Adenovirus-MVA Immunisation, 2013.
5. Schwartz, L., et al., A review of malaria vaccine clinical projects based on the WHO rainbow table. Malar J, 2012. 11: p. 11.
6. Clyde, D. F., Immunization of man against falciparum and vivax malaria by use of attenuated sporozoites. Am J Trop Med Hyg, 1975. 24(3): p. 397-401.
7. Clyde, D. F., et al., Immunization of man against sporozite-induced falciparum malaria. Am J Med Sci, 1973. 266(3): p. 169-77.
8. Hoffman, S. L., et al., Protection of humans against malaria by immunization with radiation-attenuated Plasmodium falciparum sporozoites. J Infect Dis, 2002. 185(8): p. 1155-64.
9. Nussenzweig, R. S., et al., Protective immunity produced by the injection of x-irradiated sporozoites of Plasmodium berghei. Nature, 1967. 216(5111): p. 160-2.
10. Rieckmann, K. H., et al., Use of attenuated sporozoites in the immunization of human volunteers against falciparum malaria. Bull World Health Organ, 1979. 57 Suppl 1: p. 261-5.
11. Rieckmann, K. H., et al., Letter: Sporozoite induced immunity in man against an Ethiopian strain of Plasmodium falciparum. Trans R Soc Trop Med Hyg, 1974. 68(3): p. 258-9.
12. Seder, R. A., et al., Protection Against Malaria by Intravenous Immunization with a Nonreplicating Sporozoite Vaccine. Science, 2013.
13. Doolan, D. L. and S. L. Hoffman, IL-12 and NK cells are required for antigen-specific adaptive immunity against malaria initiated by CD8+ T cells in the Plasmodium yoelii model. J Immunol, 1999. 163(2): p. 884-92.
14. Hoffman, S. L., et al., Sporozoite vaccine induces genetically restricted T cell elimination of malaria from hepatocytes. Science, 1989. 244(4908): p. 1078-81.
15. Khusmith, S., M. Sedegah, and S. L. Hoffman, Complete protection against Plasmodium yoelii by adoptive transfer of a CD8− cytotoxic T-cell clone recognizing sporozoite surface protein 2. Infect Immun, 1994. 62(7): p. 2979-83.
16. Rodrigues, M. M., et al., CD8+ cytolytic T cell clones derived against the Plasmodium yoelii circumsporozoite protein protect against malaria. Int Immunol, 1991. 3(6): p. 579-85.
17. Romero, P., et al., Cloned cytotoxic T cells recognize an epitope in the circumsporozoite protein and protect against malaria. Nature, 1989. 341(6240): p. 323-6.
18. Schofield, L., et al., Gamma interferon, CD8+ T cells and antibodies required for immunity to malaria sporozoites. Nature, 1987. 330(6149): p. 664-6.
19. Seguin, M. C., et al., Induction of nitric oxide synthase protects against malaria in mice exposed to irradiated Plasmodium berghei infected mosquitoes: involvement of interferon gamma and CD8+ T cells. J Exp Med, 1994. 180(1): p. 353-8.
20. Tarun, A. S., et al., Protracted sterile protection with Plasmodium yoelii pre-erythrocytic genetically attenuated parasite malaria vaccines is independent of significant liver-stage persistence and is mediated by CD8+ T cells. J Infect Dis, 2007. 196(4): p. 608-16.
21. Trimnell, A., et al., Genetically attenuated parasite vaccines induce contact-dependent CD8+ T cell killing of Plasmodium yoelii liver stage-infected hepatocytes. J Immunol, 2009. 183(9): p. 5870-8.
22. Weiss, W. R., et al., A T cell clone directed at the circumsporozoite protein which protects mice against both Plasmodium yoelii and Plasmodium berghei. J Immunol, 1992. 149(6): p. 2103-9.
23. Weiss, W. R. and C. G. Jiang, Protective CD8+ T lymphocytes in primates immunized with malaria sporozoites. PLoS One, 2012. 7(2): p. e31247.
24. Weiss, W. R., et al., Cytotoxic T cells recognize a peptide from the circumsporozoite protein on malaria-infected hepatocytes. J Exp Med, 1990. 171(3): p. 763-73.
25. Weiss, W. R., et al., CD8+ T cells (cytotoxic/suppressors) are required for protection in mice immunized with malaria sporozoites. Proc Natl Acad Sci USA, 1988. 85(2): p. 573-6.
26. Ferreira, A., et al., Inhibition of development of exoerythrocytic forms of malaria parasites by gamma-interferon. Science, 1986. 232(4752): p. 881-4.
27. Schofield, L., et al., Interferon-gamma inhibits the intrahepatocytic development of malaria parasites in vitro. J Immunol, 1987. 139(6): p. 2020-5.
28. Riley, E. M. and V. A. Stewart, Immune mechanisms in malaria: new insights in vaccine development. Nat Med, 2013. 19(2): p. 168-78.
29. Chuang, I., et al., DNA prime/Adenovirus boost malaria vaccine encoding P. falciparum CSP and AMA1 induces sterile protection associated with cell-mediated immunity. PLoS One, 2013. 8(2): p. e55571.
30. Kester, K. E., et al., Randomized, double-blind, phase 2a trial of falciparum malaria vaccines RTS,S/AS01B and RTS,S/AS02A in malaria-naïve adults: safety, efficacy, and immunologic associates of protection. J Infect Dis, 2009. 200(3): p. 337-46.
31. Webster, D. P., et al., Enhanced T cell-mediated protection against malaria in human challenges by using the recombinant poxviruses FP9 and modified vaccinia virus Ankara. Proc Natl Acad Sci USA, 2005. 102(13): p. 4836-41.
32. Seder, R. A., P. A. Darrah, and M. Roederer, T-cell quality in memory and protection: implications for vaccine design. Nat Rev Immunol, 2008. 8(4): p. 247-58.
33. Depinay, N., et al., Inhibitory effect of TNF-alpha on malaria pre-erythrocytic stage development: influence of host hepatocyte/parasite combinations. PLoS One, 2011. 6(3): p. e17464.
34. Horowitz, A., et al., Antigen-specific IL-2 secretion correlates with NK cell responses after immunization of Tanzanian children with the RTS,S/AS01 malaria vaccine. J Immunol, 2012. 188(10): p. 5054-62.
35. Lumsden, J. M., et al., Protective immunity induced with the RTS,S/AS vaccine is associated with IL-2 and TNF-alpha producing effector and central memory CD4 T cells. PLoS One, 2011. 6(7): p. e20775.
36. Roestenberg, M., et al., Long-term protection against malaria after experimental sporozoite inoculation: an open-label follow-up study. Lancet, 2011. 377(9779): p. 1770-6.
37. Betts, M. R., et al., Sensitive and viable identification of antige-specific CD8+ T cells by a flow cytometric assay for degranulation. J Immunol Methods, 2003. 281(1-2): p. 65-78.
38. Cohen, S., G. I. Mc, and S. Carrington, Gamma-globulin and acquired immunity to human malaria. Nature, 1961. 192: p. 733-7.
39. Mlambo, G., J. Maciel, and N. Kumar, Murine model for assessment of Plasmodium falciparum transmission-blocking vaccine using transgenic Plasmodium berghei parasites expressing the target antigen Pfs25. Infect Immun, 2008. 76(5): p. 2018-24.
40. Ramjanee, S., et al., The use of transgenic Plasmodium berghei expressing the Plasmodium vivax antigen P25 to determine the transmission-blocking activity of sera from malaria vaccine trials. Vaccine, 2007. 25(5): p. 886-94.
41. Cao, Y., D. Zhang, and W. Pan, Construction of transgenic Plasmodium berghei as a model for evaluation of blood-stage vaccine candidate of Plasmodium falciparum chimeric protein 2.9. PLoS One, 2009. 4(9): p. e6894.
42. Espinosa, D. A., et al., Development of a Chimeric Plasmodium berghei Strain Expressing the Repeat Region of the P. vivax Circumsporozoite Protein for In Vivo Evaluation of Vaccine Efficacy. Infect Immun, 2013. 81(8): p. 2882-7.
43. Persson, C., et al., Cutting edge: a new tool to evaluate human pre-erythrocytic malaria vaccines: rodent parasites bearing a hybrid Plasmodium falciparum circumsporozoite protein. J Immunol, 2002. 169(12): p. 6681-5.
44. Porter, M. D., et al., Transgenic parasites stably expressing full-length Plasmodium falciparum circumsporozoite protein as a model for vaccine down-selection in mice using sterile protection as an endpoint. Clin Vaccine Immunol, 2013. 20(6): p. 803-10.
45. Tewari, R., et al., Function of region I and II adhesive motifs of Plasmodium falciparum circumsporozoite protein in sporozoite motility and infectivity. J Biol Chem, 2002. 277(49): p. 47613-8.
46. Mueller, A. K., et al., Plasmodium liver stage developmental arrest by depletion of a protein at the parasite-host interface. Proc Natl Acad Sci USA, 2005. 102(8): p. 3022-7.
47. Reyes-Sandoval, A., et al., Prime-boost immunization with adenoviral and modified vaccinia virus Ankara vectors enhances the durability and polyfunctionality of protective malaria CD8+ T-cell responses. Infect Immun, 2010. 78(1): p. 145-53.
48. Bruna-Romero, O., et al., Complete, long-lasting protection against malaria of mice primed and boosted with two distinct viral vectors expressing the same plasmodial antigen. Proc Natl Acad Sci USA, 2001. 98(20): p. 11491-6.
49. Gessner, A., H. Blum, and M. Rollinghoff, Differential regulation of IL-9-expression after infection with Leishmania major in susceptible and resistant mice. Immunobiology, 1993. 189(5): p. 419-35.
50. Mills, C. D., et al., M-1/M-2 macrophages and the Th1/Th2 paradigm. J Immunol, 2000. 164(12): p. 6166-73.
51. Schulte, S., G. K. Sukhova, and P. Libby, Genetically programmed biases in Th1 and Th2 immune responses modulate atherogenesis. Am J Pathol, 2008. 172(6): p. 1500-8.
52. Watanabe, H., et al., Innate immune response in Th1-and Th2-dominant mouse strains. Shock, 2004. 22(5): p. 460-6.
53. Biswas, S., et al., Recombinant viral-vectored vaccines expressing Plasmodium chabaudi AS apical membrane antigen 1: mechanisms of vaccine-induced blood-stage protection. J Immunol, 2012. 188(10): p. 5041-53.
54. Draper, S. J., et al., Effective induction of high-titer antibodies by viral vector vaccines. Nat Med, 2008. 14(8): p. 819-21.
55. Bergmann-Leitner, E. S., et al., Immunization with pre-erythrocytic antigen Ce1TOS from Plasmodium falciparum elicits cross-species protection against heterologous challenge with Plasmodium berghei. PLoS One, 2010. 5(8): p. e12294.
56. Bergmann-Leitner, E. S., et al., Cellular and humoral immune effector mechanisms required for sterile protection against sporozoite challenge induced with the novel malaria vaccine candidate Ce1TOS. Vaccine, 2011. 29(35): p. 5940-9.
57. Jaffe, R. I., G. H. Lowell, and D. M. Gordon, Differences in susceptibility among mouse strains to infection with Plasmodium berghei (ANKA clone) sporozoites and its relationship to protection by gamma-irradiated sporozoites. Am J Trop Med Hyg, 1990. 42(4): p. 309-13.
58. Scheller, L. F., R. A. Wirtz, and A. F. Azad, Susceptibility of different strains of mice to hepatic infection with Plasmodium berghei. Infect Immun, 1994. 62(11): p. 4844-7.
59. Brando, C., et al., Murine immune responses to liver-stage antigen 1 protein FMP011, a malaria vaccine candidate, delivered with adjuvant AS01B or AS02A. Infect Immun, 2007. 75(2): p. 838-45.
60. Sauzet, J. P., et al., DNA immunization by Plasmodium falciparum liver-stage antigen 3 induces protection against Plasmodium yoelii sporozoite challenge. Infect Immun, 2001. 69(2): p. 1202-6.
61. Ferraro, B., et al., Inducing humoral and cellular responses to multiple sporozoite and liver stage malaria antigens using pDNA. Infect Immun, 2013.
62. O'Hara, G. A., et al., Clinical assessment of a recombinant simian adenovirus ChAd63: a potent new vaccine vector. J Infect Dis, 2012. 205(5): p. 772-81.
63. Reyes-Sandoval, A., et al., CD8+ T effector memory cells protect against liver-stage malaria. J Immunol, 2011. 187(3): p. 1347-57.
64. Roestenberg, M., et al., Protection against a malaria challenge by sporozoite inoculation. N Engl J Med, 2009. 361(5): p. 468-77.
65. Draper, S. J., M. G. Cottingham, and S. C. Gilbert, Utilizing poxviral vectored vaccines for antibody induction-Progress and prospects. Vaccine, 2013. 31(39): p. 4223-30.
66. Burbelo, P. D., et al., Antibody profiling by Luciferase Immunoprecipitation Systems (LIPS). J Vis Exp, 2009(32).
67. Burbelo, P. D., R. Goldman, and T.L. Mattson, A simplified immunoprecipitation method for quantitatively measuring antibody responses in clinical sera samples by using mammalian-produced Renilla luciferase-antigen fusion proteins. BMC Biotechnol, 2005. 5: p. 22.
68. Limbach, K., et al., Identification of two new protective pre-erythrocytic malaria vaccine antigen candidates. Malar J, 2011. 10: p. 65.
69. Bergmann-Leitner, E. S., et al., Self-adjuvanting bacterial vectors expressing pre-erythrocytic antigens induce sterile protection against malaria. Front Immunol, 2013. 4: p. 176.
70. Leitner, W. W., E. S. Bergmann-Leitner, and E. Angov, Comparison of Plasmodium berghei challenge models for the evaluation of pre-erythrocytic malaria vaccines and their effect on perceived vaccine efficacy. Malar J, 2010. 9: p. 145.
71. Douglas, A. D., et al., Tailoring subunit vaccine immunogenicity: maximizing antibody and T cell responses by using combinations of adenovirus, poxvirus and protein-adjuvant vaccines against Plasmodium falciparum MSP1. Vaccine, 2010. 28(44): p. 7167-78.
72. Draper, S. J., et al., Enhancing blood-stage malaria subunit vaccine immunogenicity in rhesus macaques by combining adenovirus, poxvirus, and protein-in-adjuvant vaccines. J Immunol, 2010. 185(12): p. 7583-95.
73. Butler, N. S., et al., Superior antimalarial immunity after vaccination with late liver stage-arresting genetically attenuated parasites. Cell Host Microbe, 2011. 9(6): p. 451-62.
74. Douradinha, B., et al., Genetically attenuated P36p-deficient Plasmodium berghei sporozoites confer long-lasting and partial cross-species protection. Int J Parasitol, 2007. 37(13): p. 1511-9.
75. Mauduit, M., et al., A role for immune responses against non-CS components in the cross-species protection induced by immunization with irradiated malaria sporozoites. PLoS One, 2009. 4(11): p. e7717.
76. Siau, A., et al., Temperature shift and host cell contact up-regulate sporozoite expression of Plasmodium falciparum genes involved in hepatocyte infection. PLoS Pathog, 2008. 4(8): p. e1000121.
77. Hill, A. V., et al., Molecular analysis of the association of HLA-B53 and resistance to severe malaria. Nature, 1992. 360(6403): p. 434-9.
78. John, C. C., et al., Cytokine responses to Plasmodium falciparum liver-stage antigen 1 vary in rainy and dry seasons in highland Kenya. Infect Immun, 2000. 68(9): p. 5198-204.
79. Krzych, U., et al., T lymphocytes from volunteers immunized with irradiated Plasmodium falciparum sporozoites recognize liver and blood stage malaria antigens. J Immunol, 1995. 155(8): p. 4072-7.
80. Kurtis, J. D., et al., Interleukin-10 responses to liver-stage antigen 1 predict human resistance to Plasmodium falciparum. Infect Immun, 1999. 67(7): p. 3424-9.
81. Luty, A. J., et al., Parasite antigen-specific interleukin-10 and antibody reponses predict accelerated parasite clearance in Plasmodium falciparum malaria. Eur Cytokine Netw, 1998. 9(4): p. 639-46.
82. Schneider, J., et al., Enhanced immunogenicity for CD8+ T cell induction and complete protective efficacy of malaria DNA vaccination by boosting with modified vaccinia virus Ankara. Nat Med, 1998. 4(4): p. 397-402.
83. Daubersies, P., et al., Protection against Plasmodium falciparum malaria in chimpanzees by immunization with the conserved pre-erythrocytic liver-stage antigen 3. Nat Med, 2000. 6(11): p. 1258-63.
84. Perlaza, B. L., et al., Protection against Plasmodium falciparum challenge induced in Aotus monkeys by liver-stage antigen-3-derived long synthetic peptides. Eur J Immunol, 2008. 38(9): p. 2610-5.
85. Reyes-Sandoval, A., et al., Mixed vector immunization with recombinant adenovirus and MVA can improve vaccine efficacy while decreasing antivector immunity. Mol Ther, 2012. 20(8): p. 1633-47.
86. Reyes-Sandoval, A., et al., Single-dose immunogenicity and protective efficacy of simian adenoviral vectors against Plasmodium berghei. Eur J Immunol, 2008. 38(3): p. 732-41.
87. Douglas, A. D., et al., The blood-stage malaria antigen PfRH5 is susceptible to vaccine-inducible cross-strain neutralizing antibody. Nat Commun, 2011. 2: p. 601.

### 3 ASSESSMENT OF PfUIS3, PfLSA1 AND PfLSAP2 AS CANDIDATES FOR A LIVER-STAGE MALARIA VACCINE

3.1 Introduction

The results presented in section 2 described the comparative assessment of the candidates, through immunogenicity studies in multiple strains of mice and efficacy against transgenic sporozoites in Balb/c mice. The vaccines encoding PfUIS3, PfLSA1 and PfLSAP2 induced the greatest level of protection, equal to or greater than protection seen with the antigens PfTRAP and PfCSP, the two most advanced clinical vaccine antigens.

In the previous section, protection against sporozoite challenge was only assessed in Balb/c mice. The drawback of using inbred mice is that protection may be MHC restricted, and hence may not translate into efficacy in humans. Ideally, vaccines identified as protective in inbred strains should also be tested in outbred mice, where there is high genetic diversity and broad MHC expression, similar to human populations. Furthermore, immunogenicity of these candidate vaccines was only assessed in the blood and spleen, yet primed immune cells are most likely required to home to the liver to exert their effect, and the liver is also an immunological organ capable of presenting antigen to naïve T cells [22].

The work described in this section aimed to further assess the protective efficacy induced by ChAd63-MVA PfUIS3, PfLSA1 and PfLSAP2 vaccination. The first aim was to confirm protection in Balb/c mice and elucidate the mechanism of protection. The second aim was to assess efficacy in two further strains of mice, C57BL/6 (H-2^{b}) and CD-1 outbred mice, to determine whether the protection was MHC restricted. The third aim was to further assess the immune response induced by these vaccines, by identifying the immunodominant epitopes in Balb/c and C57BL/6 mice and determining whether an antigen-specific response was detectable in the liver prior to challenge. A model was also available to assess the presence of HLA-A2 restricted epitopes within these antigens: transgenic mice expressing HLA-A2 [28]. HLA-A2 is a common MHC type in the general human population [29], and hence finding an HLA-A2 restricted epitope would suggest there is potential for the efficacy of these vaccines in mice to translate into humans. A probable clinical vaccination regimen using these antigens would be a multi-component malaria vaccine; therefore, the final aim of this section was the assessment of antigen interference or competition if these vaccines were used in combination with each other, or with the leading viral vectored vaccine ME-TRAP.

3.2 Results

3.2.1 Further Assessment of ChAd63-MVA PfUIS3 as a Candidate Vaccine

3.2.1.1 Confirmation of Protection in Balb/c Mice

To confirm PfUIS3 vaccination results in protection in Balb/c mice, two further independent challenges were performed using P. berghei transgenic parasites expressing P. falciparum UIS3 (PbPfUIS3). In both repeat challenges, a significant difference was confirmed between vaccinated and naïve control mice (p=0.0001 and p<0.0001, respectively, Log-rank (Mantel-Cox) Test) (FIG. 12 A and B). The protection largely presented as a delay in time to 1% parasitaemia, with a median of 7.3 days in vaccinated mice compared to 5.5 days in control mice in the first experiment (p=0.0011), and 6.8 compared to 5.1 days in the second (p=0.0001). Two out of seven mice (26%) were also sterilely protected in the first experiment, and one out of eight (12.5%) in the second. Mice were considered sterilely protected if they were slide-negative at fourteen days post-challenge. As there was no significant difference between experiments in the survival of naïve control mice, the results from the three experiments were combined (FIG. 12C). Overall, four out of 22 mice (18%) were sterilely protected with the rest exhibiting a delay in the time to 1% parasitaemia (p<0.0001). Analysis after removing the sterilely protected mice indicated a median time to 1% parasitaemia of 7.064 days in vaccinated mice compared to 5.315 days in naïve control mice (p<0.0001).

3.2.1.2 Protection in Balb/c Mice is Dependent Upon CD8⁺ T Cells

To assess the mechanism of protection, two methods were employed: in vivo depletion of either CD4⁺ or CD8⁺ T cells in mice vaccinated with ChAd63-MVA PfUIS3, or the adoptive transfer of CD4⁻ or CD8⁺ enriched splenocytes from ChAd63-MVA PfUIS3 vaccinated mice into naïve mice, followed by PbPfUIS3 sporozoite challenge. CD4⁺ or CD8⁺ T cells were depleted by injection of monoclonal antibodies (mAb) into vaccinated mice; 100 μl g injected intraperitoneal on three consecutive days depleted 100% of either cell population (assessed in the blood four days post-challenge). No differences were found in the survival of control vaccinated mice and mice injected with an IgG control mAb (FIG. 13). In the absence of CD8⁺ T cells no significant difference in survival compared to naïve controls was observed, while a significant difference to control vaccinated mice was seen (p=0.0001, Log-rank (Mantel-Cox) Test). The median time to 1% parasitaemia was reduced from 7.284 days in control-vaccinated mice to 5.57 days in CD8⁺ depleted mice (p=0.0008). CD4⁺ depletion also significantly reduced efficacy compared to control vaccinated mice (p=0.0007), however this regimen still provided some degree of protection compared to naïve mice (median of 6.45 days compared to 5.47 days in naïve controls, p<0.0001).

3.2.1.3 ChAd63-MVA PfUIS3 Vaccination Also Provides Protection Against Sporozoite Challenge in C57BL/6 Mice

To determine whether protection against sporozoite challenge was specific to Balb/c mice (i.e. restricted by H-2^{d}), efficacy was also assessed in C57BL/6 mice (H-2^{b}) and CD-1 mice (an outbred strain). Two challenge experiments were performed in C57BL/6 mice with PbPfUIS3, both inducing a significant difference in survival compared to naïve control mice (both experiments p<0.0001, Log-rank (Mantel-Cox) Test). As the survival of the naïve controls differed significantly between the two experiments they could not be combined.

Representative results from the second experiment are shown (FIG. 14B). In the first experiment two mice were sterilely protected (25%) and there was a significant delay in the time to 1% parasitaemia for the remaining mice (7.24 versus 5.45 days in naïve controls, p=0.0003); in the second, three were sterilely protected (37.5%) and again there was a significant delay in the time to 1% parasitaemia for the remaining mice (7.58 versus 4.69 days in naïve controls, p=0.0008). A high level of antigen-specific CD8⁺ T cells were measured prior to challenge, with a median of 4.8% CD8⁺ IFNγ⁺ (FIG. 14A); correlations were performed for all immunogenicity measures (including polyfunctional CD8⁺ T cells and antibody levels) and a significant negative correlation was identified between the time to 1% parasitaemia and CD8⁺ IL-2 secreting cells (Spearman r=−0.756, p=0.0368) (FIG. 14C). This correlation was not identified in the first C57BL/6 challenge experiment.

One challenge experiment was performed in the outbred laboratory strain CD-1. Whilst an immune response was induced (median CD8⁺ IFNγ⁺ of 0.9%, FIG. 15A), no significant difference in efficacy was observed between vaccinated and naïve control mice (median time to 1% parasitaemia of 6.77 days in vaccinated mice compared to 5.67 days in control mice, FIG. 15B), despite an initial trend. As increased variability is expected in outbred mice, future experiments should include greater sample sizes. Despite the absence of any protective efficacy, a significant positive correlation was identified between the time to 1% parasitaemia of vaccinated mice and the percentage of both CD8⁺ cells producing IFNγ (Spearman r=0.7306, p=0.0368, FIG. 15C) or TNFα (Spearman r=0.7857, p=0.0279).

3.2.2 Further Assessment of ChAd63-MVA PfLSA1 as a Candidate Vaccine

3.2.2.1 Confirmation of Sterile Protection in Balb/c Mice

To confirm PfLSA1 vaccination results in sterile protection in Balb/c mice, an independent repeat challenge was performed using transgenic P. berghei parasites expressing P. falciparum LSA1 (PbPfLSA1). A significant difference was confirmed between vaccinated and naïve control mice (p<0.0001, Log-rank (Mantel-Cox) Test) (FIG. 17A), with six out of eight mice sterilely protected (75%). As there was no significant difference between the repeat and original experiment in the survival of naïve control mice, the results from the two experiments were combined (FIG. 17B), resulting in thirteen out of sixteen mice (81.25%) sterilely protected from malaria after PfLSA1 vaccination (p<0.0001).

3.2.2.2 Protection in Balb/c Mice is Dependent Upon CD8⁺ T cells

As thirteen out of sixteen mice were sterilely protected, and hence given the arbitrary value of ‘14’ in the time to 1% parasitaemia analysis, correlations with immune subsets are statistically challenging. Stratifying the mice into ‘delayed’ and ‘sterile protection’ also provided statistical difficulty, given only three mice were delayed. Performing such analysis identified no significant differences between mice with a delay in the time to 1% parasitaemia or those sterilely protected when any immune subsets were assessed. PfLSA1 vaccination also induced polyfunctional antigen-specific CD8⁺ T cells, with approximately 50-75% producing both IFNγ and TNFα post-boost in the blood and spleen. Assessing all permutations of polyfunctionality found no immune subsets that differed significantly between delayed and protected mice.

To overcome this statistical limitation, the effect of CD8⁺ or CD4⁺ T cells was assessed by in vivo depletions of each of these subsets prior to transgenic sporozoite challenge. CD4⁻ or CD8⁺ T cells were depleted by injection of monoclonal antibodies into vaccinated mice; 100 μl g injected intraperitoneal on three consecutive days depleted 100% of either cell population (assessed in the blood four days post-challenge). No differences were found in the survival of PfLSA1 control vaccinated mice and mice depleted with an IgG control mAb (FIG. 18). CD8⁻ depletion reduced the protection induced by PfLSA1 vaccination, as shown by no significant difference in survival compared to naïve mice and a significant difference compared to PfLSA1 vaccinated control mice (p=0.0027, Log-rank (Mantel-Cox) Test). However, one mouse was sterilely protected. CD4⁺ depletion reduced the protection induced by PfLSA1 vaccination, as shown by a significant difference in survival compared to PfLSA1 control vaccinated mice (p=0.026), however these mice could still induce a significant level of protection compared to naïve mice (p=0.0003).

3.2.2.3 ChAd63-MVA PfLSA I Vaccination Also Provides Protection Against Sporozoite Challenge in CD-1 Outbred Mice

To determine whether protection against transgenic sporozoite challenge was specific to Balb/c mice (i.e. restricted by H-2^{d}), efficacy was also assessed in C57BL/6 mice (H-2^{b}) and CD-1 outbred mice. Since no cellular immune response was observed after ChAd63-MVA PfLSA1 vaccination of C57BL/6 mice, it was not surprising that PbPfLSA1 sporozoite challenge resulted in no protection in this strain (FIG. 19). PfLSA1 vaccination was able to induce an immune response in CD-1 outbred mice (FIG. 20A), with a median CD8⁺ IFNγ⁺ response of 1.13%, TNFα⁺ of 1.2% and CD107a⁺ of 5.5%. Upon challenge with transgenic PbPfLSA1 sporozoites, seven out of eight mice were sterilely protected (87.5%, p<0.0001, Log-rank (Mantel-Cox) Test) (FIG. 20B). As for PfLSA1 efficacy in Balb/c mice, it was difficult to assess correlates of protection given the majority of mice did not develop malaria. In this case, as only one mouse was not sterilely protected, it was not possible to perform analysis of significant differences between delayed and sterile protection.

3.2.2.4 Further Assessment of Immunogenicity Induced by ChAd63-MVA PfLSA1 Vaccination

As significant protective efficacy was identified in Balb/c mice, it was of interest to know which epitopes were associated with protective responses and whether it was possible to detect an HLA-A2-restricted immune response. Epitope mapping was conducted in Balb/c and HHD (HLA-A2 transgenic) mice by spleen IFNγ ELISpot to individual peptides covering the entire PfLSA1 sequence. Immunodominant responses in Balb/c mice were identified to peptides 20 (aa918 to 937) and 40 (aa1118 to 1137), with three further subdominant responses. No HLA-A2 restricted epitopes were identified in HHD mice.

Immunogenicity was assessed in liver mononuclear cells of PfLSA1 vaccinated mice as for PfUIS3 vaccinated mice. A low, but detectable, level of PfLSA1-specific cells were observed (FIG. 21), with medians of 0.35% CD8⁻ IFNγ⁺, 0.38% TNFα⁺ and 0.94% CD107a⁺. This was significantly lower than levels seen in spleens from the same mice (p<0.0001, two-way ANOVA). The values were considered too low to reliably assess the expression of memory cell markers.

3.2.3 Further Assessment of ChAd63-MVA PfLSAP2 as a Candidate Vaccine 3.2.3.1 Confirmation of Sterile Protection in Balb/c Mice

To confirm PfLSAP2 vaccination results in sterile protection in Balb/c mice, an independent repeat challenge was performed with transgenic P. berghei parasites expressing P. falciparum LSAP2 (PbPfLSAP2). A significant difference was confirmed between vaccinated and naïve control mice (p=0.0002, Log-rank (Mantel-Cox) Test) (FIG. 22A), with five out of eight mice sterilely protected (62.5%). As there was no significant difference in the survival of naïve control mice between the repeat and original experiment, the results from the two experiments were combined (FIG. 22B). Overall, twelve out of sixteen mice (75%) were sterilely protected (p<0.0001).

PfLSAP2 vaccination in Balb/c mice resulted in both a moderate cellular immune response (median 446 SFC per million splenocytes post-boost) and a detectable antibody response (median log luminescence of 6). No correlates of protection could be identified for cellular or humoral immunogenicity, nor was a significant difference seen when grouping vaccinated mice into ‘delayed’ or ‘sterile protection’. As for PfLSA1, statistical analysis was difficult, given the low numbers of mice who were not protected. Polyfunctionality was also assessed, and unlike PfUIS3 or PfLSA1 vaccination, most CD8⁺ T cells were single cytokine producers (IFNγ or TNFα). All permutations of polyfunctionality were analyzed for differences between mice sterilely protected and those delayed, but no differences were found. As PfLSAP2 vaccination induced a cellular immune response in C57BL/6 mice (median 892 SFC per million splenocytes) but no antibody response, protective efficacy was then assessed in this strain to determine whether protection was likely mediated through cellular or humoral immunity.

3.2.3.2 ChAd63-MVA PfLSAP2 Vaccination Does Not Induce Protection Against Sporozoite Challenge in C57BL/6 Mice

C57BL/6 mice were vaccinated with PfLSAP2 in the standard prime-boost regimen and efficacy tested by transgenic PbPfLSAP2 sporozoite challenge. Despite a moderate cellular immune response (median 3.4% of CD8⁺ T cells producing IFNγ, 3.6% TNFα and 3.3% CD107a) (FIG. 23A), vaccinated mice were not protected from sporozoite challenge (FIG. 23B). The cellular immune response was comparable to PfLSAP2 vaccination in Balb/c mice, except that a greater proportion of antigen-specific CD8⁺ T cells were double cytokine producers (approximately 75% post-boost in the spleen). No antibodies were detected in these mice seven days post-MVA boost (pre-challenge).

3.2.4 Comparison of the Protective Efficacy Induced by PfUIS3, PfLSA1 and PfLSAP2 Vaccination and Assessment of Competition When Combining Vaccines

PfUIS3, PfLSA1 and PfLSAP2 were all identified as promising candidate antigens for a pre-erythrocytic malaria vaccine due to the efficacy provided in Balb/c mice. As indicated in Table 3.1, PfUIS3 and PfLSA1 subsequently provided protection in another strain of mice, either C57BL/6 or CD-1, but not both. PfLSAP2 vaccination did not provide protection in C57BL/6 mice, but efficacy is still to be assessed in CD-1 outbred mice. PfLSA1 was identified as a promising candidate due to protection in outbred mice, given these mice are more representative of an outbred human population. These candidate antigens could be used as part of a multi-component malaria vaccine, either in combination with the current leading viral vectored vaccine ME-TRAP, or in combination with each other.

TABLE 3.1
Comparison of protective efficacy induced by PfUIS3, PfLSA1
and PfLSAP2 in three different strains of mice.

| Antigen | Balb/c | C57BL/6 | CD-1 | Mechanism |
|---|---|---|---|---|
| PfUIS3 | 18% sterile | 37.5% sterile | No | CD8⁺ T |
| | protection, | protection, | protection | cells |
| | significant | significant | | |
| | delay | delay | | |
| PfLSA1 | 81.25% sterile | No protection | 87.5% sterile | CD8⁺ T |
| | protection | | protection | cells |
| PfLSAP2 | 75% sterile | No protection | Not assessed | Unknown |
| | protection | | | |

To assess whether combining each of the vaccines with ME-TRAP would affect the immunogenicity of each individual vaccine, C57BL/6 mice were vaccinated with ME-TRAP in combination with either PfUIS3 or PfLSAP2. The effect of PfUIS3 and PfLSAP2 combination vaccination was also assessed. C57BL/6 mice were chosen as the ME string contains the strong P. berghei Pb9 H-2d-restricted epitope from CSP [34], and hence immunogenicity measured in Balb/c mice would reflect the effect of competition on P. berghei CSP rather than P. falciparum TRAP. Vaccinating with two vaccines did not significantly reduce or increase the immunogenicity of either vaccine, compared to administration of either vaccine alone (FIG. 25).

As PfLSA1 does not induce an immune response in C57BL/6 mice, it could not be assessed in the experiment outlined above. Instead, to circumvent the Pb9 epitope, the vaccine TRIP was used in Balb/c mice. TRIP is codon optimized P. falciparum 3D7 TRAP, without the ME string (TRAP sequence is derived from the P. falciparum T9/96 strain). Vaccinating with both TRIP and PfLSA1 together did not significantly reduce or increase the immunogenicity of either vaccine compared to administration of either vaccine alone (FIG. 26).

### 4. PROTECTIVE EFFICACY OF THE CANDIDATES VACCINES IN CD-1 OUTBRED MICE

The efficacy of P. falciparum vaccine candidates in CD-1 outbred mice following the standard prime-boost, eight-week interval ChAd63-MVA vaccination regime was assessed. PfLSA1 vaccination protected 7/8 (87.5%) CD-1 mice from chimeric sporozoite challenge, resulting in a significant level of survival compared to naïve controls (p<0.0001, Log-Rank (Mantel-Cox) Test). PfLSAP2 protected 7/10 (70%) CD-1 mice challenged with chimeric sporozoites, a significant level of protection compared to naïve controls (p=0.0009, Log-Rank (Mantel-Cox) Test. PfUIS3 vaccination was unable to significantly protect CD-1 mice against challenge, despite an initial trend (median of 6.77 days compared to 5.67 days in naïve controls).

As PfCSP and PfTRAP acted as our compactor vaccines, we also assessed their efficacy in CD-1 mice, in order to bypass the MHC restriction and immunodominance observed in inbred strains of mice. Following the standard ChAd63-MVA regimen, PfCSP was able to protect 3/9 (33.3%) CD-1 mice and induced a delay in time to 1% parasitaemia by a median 0.48 days (overall p=0.001, Log-Rank (Mantel-Cox) Test) (Table 4), similar to the induced efficacy in BALB/c mice. PfTRAP was able to protect 3/10 (30%) CD-1 mice but did not cause a delay in the time to 1% parasitaemia in those for which sterile protection was not induced (p=0.02, Log-Rank (Mantel-Cox) Test) (FIG. 27). PfTRAP provided protection against chimeric sporozoite challenge in CD-1 mice, this was despite any sterile protection observed in BALB/c mice. Therefore we subsequently assessed efficacy of the remaining antigens (those modestly protective, or non-protective in BALB/c) in CD-1 mice to ensure no potential candidates were missed. Both PfFalstatin and PfLSA3, which both had provided a small degree of protection in BALB/c mice, a degree of protection was maintained in CD-1 mice, with 1/10 (10%) sterilily protected and the rest exhibiting a significant delay in the time to 1% parasitaemia (median delay of 0.97 days, p<0.0001, Log-Rank (Mantel-Cox) Test). For PfLSA3, this effect was not maintained as no protection was observed in CD-1 mice. Of those vaccines that did not induce protection in BALB/c mice, PfCe1TOS, PfLSAP1 and PfETRAMP5, none subsequently induced a statistically significant level of protection in CD-1 mice (Table 4).

The rank/order of the new P. falciparum antigens using the P. falciparum expressing P. berghei transgenic parasite challenge model is presented in FIG. 28 where both PfLSA1 and PfLSAP2 antigens have shown high level of efficacy in mice which is greater than efficacy achieved with the leading vaccine candidates PfTRAP and PfCSP in both inbred Balb/c and outbred CD-1 mice.

TABLE 4
Sterile protection and median delay induced by ChAd63-
MVA P. falciparum vaccines in CD-1 mice.

| | Protection | |
|---|---|---|
| Vaccine | (%)¹ | Median delay² |
| PfLSA1 | 87.5**** | 2 |
| PfLSA3³ | 0 | 0.22 |
| PfCelTOS | 0 | 0.28 |
| PfUIS3 | 0 | 1.1 |
| PfLSAP1 | 30 | 0 |
| PfLSAP2 | 70*** | 0.29 |
| PfETRAMP5 | 10 | 0 |
| PfFalstatin | 10**** | 0.97**** |
| PfCSP | 33.3** | 0.48* |
| PfTRAP | 30* | 0.03 |

¹Percentage of mice that received sterile protection from vaccination after challenge with 1000 chimeric sporozoites i.v., n = 8-10.
²The median delay (days) in time to 1% parasitaemia, calculated by: (time to 1% of vaccinee) − (average time to 1% of naïve controls). The difference in survival was generated using Kaplan-Meier survival curves with statistical significance assessed using the Log-Rank (Mantel-Cox) Test,
*p < 0.05-0.01
**p < 0.01-0.001
***p < 0.001
****p < 0.0001.
For the median delay, statistical significance was assessed after the removal of uninfected mice (sterile protection).
³For PfLSA3 challenge, the chimeric sporozoite dose was increased to 2000 sporozoites per mouse in order to infect all naïve controls.

### 5. SUMMARY

In summary, the results support PfLSA1, PfUIS3, PfLSAP2 and PfI0580c expressed in viral vectors, especially simian adenovirus and MVA, as candidate vaccines. PfUIS3 vaccination was able to induce similar levels of efficacy in two inbred strains of mice, most likely through the action of CD8⁺ T cells on liver-stage parasites. There was a trend towards protection in outbred mice, which may be achievable if the percentage of antigen-specific cells is increased. PfUIS3 is located in the PVM, providing support that this protein could be exported into the hepatocyte cytoplasm and presented on the cell surface. These are the first results showing the promise of PfUIS3 alone, not just in combination. Whilst PfLSAP2 induced a high degree of sterile protection in Balb/c mice, this is likely either H-2^{d}-restricted or antibody-mediated. These results represent the first assessment of PfLSAP2 as a vaccine candidate, and warrant further investigation. PfLSA1 was identified as the strongest candidate, with almost complete sterile protection in outbred mice. PfLSA1 is indispensible for liver-stage infection, has consistently been associated with protection in natural immunity and these results strongly suggest it is presented on the hepatocyte cell-surface as a target of CD8⁺ T cells. These results also highlight the value of transgenic parasites, as both PfLSA1 and PfLSAP2 contain no murine homologs and hence efficacy has not previously been possible to assess in mice.

### REFERENCES

1. Kaiser, K., et al., Differential transcriptome profiling identifies Plasmodium genes encoding pre-erythrocytic stage-specific proteins. Mol Microbiol, 2004. 51(5): p. 1221-32.
2. Matuschewski, K., et al., Infectivity-associated changes in the transcriptional repertoire of the malaria parasite sporozoite stage. J Biol Chem, 2002. 277(44): p. 41948-53.
3. Mikolajczak, S. A., et al., L-FABP is a critical host factor for successful malaria liver stage development. Int J Parasitol, 2007. 37(5): p. 483-9.
4. Mueller, A. K., et al., Genetically modified Plasmodium parasites as a protective experimental malaria vaccine. Nature, 2005. 433(7022): p. 164-7.
5. Sharma, A., et al., Crystal structure of soluble domain of malaria sporozoite protein UIS3 in complex with lipid. J Biol Chem, 2008. 283(35): p. 24077-88.
6. Favretto, F., et al., Evidence from NMR interaction studies challenges the hypothesis of direct lipid transfer from L-FABP to malaria sporozoite protein UIS3. Protein Sci, 2013. 22(2): p. 133-8.
7. Tarun, A. S., et al., Protracted sterile protection with Plasmodium yoelii pre-erythrocytic genetically attenuated parasite malaria vaccines is independent of significant liver-stage persistence and is mediated by CD8+ T cells. J Infect Dis, 2007. 196(4): p. 608-16.
8. Hoffman, B. U. and R. Chattopadhyay, Plasmodium falciparum: effect of radiation on levels of gene transcripts in sporozoites. Exp Parasitol, 2008. 118(2): p. 247-52.
9. Limbach, K., et al., Identification of two new protective pre-erythrocytic malaria vaccine antigen candidates. Malar J, 2011. 10: p. 65.
10. Siau, A., et al., Temperature shift and host cell contact up-regulate sporozoite expression of Plasmodium falciparum genes involved in hepatocyte infection. PLoS Pathog, 2008. 4(8): p. e1000121.
11. Guerin-Marchand, C., et al., A liver-stage-specific antigen of Plasmodium falciparum characterized by gene cloning. Nature, 1987. 329(6135): p. 164-7.
12. Fidock, D. A., et al., Plasmodium falciparum liver stage antigen-1 is well conserved and contains potent B and T cell determinants. J Immunol, 1994. 153(1): p. 190-204.
13. Mikolajczak, S. A., et al., Disruption of the Plasmodium falciparum liver-stage antigen-1 locus causes a differentiation defect in late liver-stage parasites. Cell Microbiol, 2011. 13(8): p. 1250-60.
14. John, C. C., et al., Cytokine responses to Plasmodium falciparum liver-stage antigen 1 vary in rainy and dry seasons in highland Kenya. Infect Immun, 2000. 68(9): p. 5198-204.
15. Krzych, U., et al., T lymphocytes from volunteers immunized with irradiated Plasmodium falciparum sporozoites recognize liver and blood stage malaria antigens. J Immunol, 1995. 155(8): p. 4072-7.
16. Kurtis, J. D., et al., Interleukin-10 responses to liver-stage antigen 1 predict human resistance to Plasmodium falciparum. Infect Immun, 1999. 67(7): p. 3424-9.
17. Luty, A. J., et al., Parasite antigen-specific interleukin-10 and antibody reponses predict accelerated parasite clearance in Plasmodium falciparum malaria. Eur Cytokine Netw, 1998. 9(4): p. 639-46.
18. Migot-Nabias, F., et al., Immune response to Plasmodium falciparum liver stage antigen-1: geographical variations within Central Africa and their relationship with protection from clinical malaria. Trans R Soc Trop Med Hyg, 2000. 94(5): p. 557-62.
19. Hill, A. V., et al., Molecular analysis of the association of HLA-B53 and resistance to severe malaria. Nature, 1992. 360(6403): p. 434-9.
20. Cummings, J. F., et al., Recombinant Liver Stage Antigen-1 (LSA-1) formulated with AS01 or AS02 is safe, elicits high titer antibody and induces IFN-gamma/IL-2 CD4+ T cells but does not protect against experimental Plasmodium falciparum infection. Vaccine, 2010. 28(31): p. 5135-44.
21. Porter, D. W., et al., A human Phase I/IIa malaria challenge trial of a polyprotein malaria vaccine. Vaccine, 2011. 29(43): p. 7514-22.
22. Jenne, C. N. and P. Kubes, Immune surveillance by the liver. Nat Immunol, 2013. 14(10): p. 996-1006.
23. Berenzon, D., et al., Protracted protection to Plasmodium berghei malaria is linked to functionally and phenotypically heterogeneous liver memory CD8+ T cells. J Immunol, 2003. 171(4): p. 2024-34.
24. Krzych, U., et al., The role of intrahepatic lymphocytes in mediating protective immunity induced by attenuated Plasmodium berghei sporozoites. Immunol Rev, 2000. 174: p. 123-34.
25. Nganou-Makamdop, K., et al., Long term protection after immunization with P. berghei sporozoites correlates with sustained IFNgamma responses of hepatic CD8+ memory T cells. PLoS One, 2012. 7(5): p. e36508.
26. Reyes-Sandoval, A., et al., Prime-boost immunization with adenoviral and modified vaccinia virus Ankara vectors enhances the durability and polyfunctionality of protective malaria CD8+ T-cell responses. Infect Immun, 2010. 78(1): p. 145-53.
27. Reyes-Sandoval, A., et al., CD8+ T effector memory cells protect against liver-stage malaria. J Immunol, 2011. 187(3): p. 1347-57.
28. Pascolo, S., et al., HLA-A2.1-restricted education and cytolytic activity of CD8(+) T lymphocytes from beta2 microglobulin (beta2m) HLA-A2.1 monochain transgenic H-2Db beta2m double knockout mice. J Exp Med, 1997. 185(12): p. 2043-51.
29. Sidney, J., et al., Practical, biochemical and evolutionary implications of the discovery of HLA class I supermotifs. Immunol Today, 1996. 17(6): p. 261-6.
30. Miller, J. L., et al., Quantitative bioluminescent imaging of pre-erythrocytic malaria parasite infection using luciferase-expressing Plasmodium yoelii. PLoS One, 2013. 8(4): p. e60820.
31. Ploemen, I. H., et al., Visualisation and quantitative analysis of the rodent malaria liver stage by real time imaging. PLoS One, 2009. 4(11): p. e7881.
32. Krzych, U., et al., Memory CD8 T cells specific for plasmodia liver-stage antigens maintain protracted protection against malaria. Front Immunol, 2012. 3: p. 370.
33. Teirlinck, A. C., et al., Longevity and composition of cellular immune responses following experimental Plasmodium falciparum malaria infection in humans. PLoS Pathog, 2011. 7(12): p. e1002389.
34. Romero, P., et al., Cloned cytotoxic T cells recognize an epitope in the circumsporozoite protein and protect against malaria. Nature, 1989. 341(6240): p. 323-6.
35. Bergmann-Leitner, E. S., et al., Immunization with pre-erythrocytic antigen Ce1TOS from Plasmodium falciparum elicits cross-species protection against heterologous challenge with Plasmodium berghei. PLoS One, 2010. 5(8): p. e12294.
36. Charoenvit, Y., et al., CD4(|) T-cell- and gamma interferon-dependent protection against murine malaria by immunization with linear synthetic peptides from a Plasmodium yoelii 17-kilodalton hepatocyte erythrocyte protein. Infect Immun, 1999. 67(11): p. 5604-14.
37. Brando, C., et al., Murine immune responses to liver-stage antigen 1 protein FMP011, a malaria vaccine candidate, delivered with adjuvant AS01B or AS02A. Infect Immun, 2007. 75(2): p. 838-45.
38. Prieur, E., et al., A Plasmodium falciparum candidate vaccine based on a six-antigen polyprotein encoded by recombinant poxviruses. Proc Natl Acad Sci USA, 2004. 101(1): p. 290-5.
39. Roestenberg, M., et al., Protection against a malaria challenge by sporozoite inoculation. N Engl J Med, 2009. 361(5): p. 468-77.
40. Roestenberg, M., et al., Long-term protection against malaria after experimental sporozoite inoculation: an open-label follow-up study. Lancet, 2011. 377(9779): p. 1770-6.
41. Ewer, K. J., Protective CD8+ T cell Immunity to Human Malaria Induced by Chimpanzee Adenovirus-MVA Immunisation, 2013.
42. Seder, R. A., et al., Protection Against Malaria by Intravenous Immunization with a Nonreplicating Sporozoite Vaccine. Science, 2013.
43. Guebre-Xabier, M., R. Schwenk, and U. Krzych, Memory phenotype CD8(+) T cells persist in livers of mice protected against malaria by immunization with attenuated Plasmodium berghei sporozoites. Eur J Immunol, 1999. 29(12): p. 3978-86.
44. Morrot, A., et al., IL-4 receptor expression on CD8+ T cells is required for the development of protective memory responses against liver stages of malaria parasites. J Exp Med, 2005. 202(4): p. 551-60.
45. Klonowski, K. D., et al., Dynamics of blood-borne CD8 memory T cell migration in vivo. Immunity, 2004. 20(5): p. 551-62.
46. Wherry, E. J., et al., Lineage relationship and protective immunity of memory CD8 T cell subsets. Nat Immunol, 2003. 4(3): p. 225-34.
47. Chuang, I., et al., DNA prime/Adenovirus boost malaria vaccine encoding P. falciparum CSP and AMA1 induces sterile protection associated with cell-mediated immunity. PLoS One, 2013. 8(2): p. e55571.
48. Schluns, K. S. and L. Lefrancois, Cytokine control of memory T-cell development and survival. Nat Rev Immunol, 2003. 3(4): p. 269-79.
49. Horowitz, A., et al., Antigen-specific IL-2 secretion correlates with NK cell responses after immunization of Tanzanian children with the RTS,S/AS01 malaria vaccine. J Immunol, 2012. 188(10): p. 5054-62.
50. Lumsden, J. M., et al., Protective immunity induced with the RTS,S/AS vaccine is associated with IL-2 and TNF-alpha producing effector and central memory CD4 T cells. PLoS One, 2011. 6(7): p. e20775.
51. Good, M. F. and D. L. Doolan, Malaria vaccine design: immunological considerations. Immunity, 2010. 33(4): p. 555-66.
52. Riley, E. M. and V. A. Stewart, Immune mechanisms in malaria: new insights in vaccine development. Nat Med, 2013. 19(2): p. 168-78.
53. Ferraro, B., et al., Inducing humoral and cellular responses to multiple sporozoite and liver stage malaria antigens using pDNA. Infect Immun, 2013.
54. Forbes, E. K., et al., Combining liver- and blood-stage malaria viral-vectored vaccines: investigating mechanisms of CD8+ T cell interference. J Immunol, 2011. 187(7): p. 3738-50.
55. Pichyangkul, S., et al., Evaluation of the safety and immunogenicity of Plasmodium falciparum apical membrane antigen 1, merozoite surface protein 1 or RTS,S vaccines with adjuvant system AS02A administered alone or concurrently in rhesus monkeys. Vaccine, 2009. 28(2): p. 452-62.
56. Sedegah, M., et al., Effect on antibody and T-cell responses of mixing five GMP-produced DNA plasmids and administration with plasmid expressing GM-CSF. Genes Immun, 2004. 5(7): p. 553-61.
57. Sedegah, M., et al., Reduced immunogenicity of DNA vaccine plasmids in mixtures. Gene Ther, 2004. 11(5): p. 448-56.
58. Grifantini, R., et al., Multi-plasmid DNA vaccination avoids antigenic competition and enhances immunogenicity of a poorly immunogenic plasmid. Eur J Immunol, 1998. 28(4): p. 1225-32.
59. Jiang, G., et al., Induction of multi-antigen multi-stage immune responses against Plasmodium falciparum in rhesus monkeys, in the absence of antigen interference, with heterologous DNA prime/poxvirus boost immunization. Malar J, 2007. 6: p. 135.
60. Jones, T. R., et al., Absence of antigenic competition in Aotus monkeys immunized with Plasmodium falciparum DNA vaccines delivered as a mixture. Vaccine, 2002. 20(11-12): p. 1675-80.
61. Pichyangkul, S., et al., Preclinical evaluation of the safety and immunogenicity of a vaccine consisting of Plasmodium falciparum liver-stage antigen 1 with adjuvant AS01B administered alone or concurrently with the RTS,S/AS01B vaccine in rhesus primates. Infect Immun, 2008. 76(1): p. 229-38.
62. Wang, R., et al., Simultaneous induction of multiple antigen-specific cytotoxic T lymphocytes in nonhuman primates by immunization with a mixture of four Plasmodium falciparum DNA plasmids. Infect Immun, 1998. 66(9): p. 4193-202.
63. Kocken, C. H., et al., Precise timing of expression of a Plasmodium falciparum-derived transgene in Plasmodium berghei is a critical determinant of subsequent subcellular localization. J Biol Chem, 1998. 273(24): p. 15119-24.

### 5. SPECT-1

5.1 PfSPECT-1 Protein as a Vaccine Candidate

Sporozoite surface proteins such as CS, TRAP, and SPECT-1 are highly involved in sporozoite movement and interaction with host cell receptors, and could induce a protective immune response [1, 7, 8, 20]. The sporozoite microneme protein essential for cell traversal, SPECT-1, is considered a potential pre-erythrocytic immune target due to the key role it plays in crossing of the malaria parasite across the dermis and the liver sinusoidal wall, prior to invasion of hepatocytes [16, 21] but they have not previously been shown to provide any protective efficacy as vaccine candidates. Several sporozoite proteins have been implicated in crossing the dermal cell barrier and subsequent migration to liver sinusoid [22],[23],[24], [25].

### 6. RESULTS

6.1 Design and Generation of PfSPECT-1 -ChAd63 and -MVA Viral Vector Vaccines

Vectored vaccines were developed using the available 3D7 P. falciparum coding sequence with the tissue plasminogen activator (tPA) leader sequence [23] added upstream, as in the clinical ME-TRAP vectors, to aid in secretion, expression and thereby immunogenicity [24-26]. Vaccine sequence was modified for mammalian codon optimization prior to cloning into the ChAd63 and MVA vectors. The size and the sequence details of PfSPECT-1 antigen are listed below. Integration and ID PCR were done and confirmed the correct insertion and integration of PfSPECT-1 antigen into the correct locus in the viral vector vaccines.

6.2 Design and Generation of PfSPECT-1 Expressing P. berghei Chimeric Parasites

Chimeric parasite expressing PfSPECT-1 protein was generated by introduction of the coding sequence of the PfSPECT-1 antigen into the silent 230p locus of the reference line P. berghei ANKA following the methodology of ‘gene insertion/marker out’ (GIMO) transfections [27]. The P. falciparum gene coding sequence was placed under control of the regulatory regions (the promoter and transcriptional terminator sequences) of the P. berghei UIS4 gene. The UIS4 gene is specifically expressed at the Plasmodium sporozoite and liver-stages [28, 29]. Genotype analyses of the cloned PfSPECT-1_{Pbuis4} (2414 cl1) chimeric line generated confirmed correct integration of the PfSPECT-1 coding sequence into the P. berghei genome. Phenotype analysis of the chimeric parasites, using an immunofluorescence assay, confirmed the expression of the P. falciparum candidates in the chimeric sporozoites (FIG. 31A). Chimeric parasite fitness and liver loads in naïve mice were assessed by their challenged with transgenic chimeric sporozoites were quantified by measuring luminescence levels of the Luciferase activity at 44 hours after infection using the IVIS 200 system (FIG. 31B).

6.3 Immunisation and Protective Efficacy Assessment of PfSPECT-1 Vaccine in Balb/c Inbred and CD-1 Outbred Mice in vivo.

Standard heterologous ChAd63-MVA prime-boost vaccination strategy was followed in this challenge experiment. Mice were vaccinated i.m. with 1×10⁸ ifu ChAd63-PfLSPECT-1 followed eight weeks later by 1×10⁷ pfu MVA-PfLSPECT-1. Mice were challenged i.v. with 1000 transgenic PfLSPECT-1_{Pbuis4} (2414 cl1) sporozoites ten days post-MVA boost, along with naive control mice. Mice were monitored daily to enable calculation of the time to 1% parasitaemia. Mice that were slide-negative at fourteen days post-challenge were considered sterilely protected. The Log-rank (Mantel-Cox) test was used to assess differences between the survival curves. PfSPECT-1 vaccination resulted in a good sterile protection level and significant delay to 1% parasitaemia. In Balb/c inbred mice (vaccinated n=8, naive n=8); PfSPECT-1 induced 37.5% sterile protection with a significant delay to 1% parasitaemia p=0.0008. While, in CD-1 outbred mice (vaccinated n=10, naive n=10), PfSPECT-1 induced 70% sterile protectTion with a significant delay to 1% parasitaemia p=0.0023 (FIG. 32). PfSPECT-1 induced higher protection level in this challenge model in comparison to our standard current leading P. falciparum malaria vaccine PfCSP which showed 31.25% and 33.3% sterile protection in Balb/c and CD-1 mice, respectively (FIG. 33).

6.4 In vitro Assessment of Blocking Activity of Serum From Mice Vaccinated with PfSPECT-1 Viral Vaccines

The inclusion of the GFP-luciferace expression cassette in PfSPECT-1_{Pbuis4} chimeric with its ability to express the GFP fluorescent protein allowed the assessment of the blocking activity of serum from mice vaccinated with PfSPECT-1 viral vaccine in vitro based on measuring the decline in the emitted GFP signal from the infected hepatocytes with the chimeric parasite in a cell culture plate in case of adding serum from vaccinated mice to it in comparison to the use of naïve mice sera. Specifically, 30,000 Huh-7 hepatocytes were seeded in 96 cell culture plate. After 12 hours; 15,000 PfSPECT-1 chimeric sporozoites were added per hepatocyte wells either mixed with sera from mice vaccinated against PfSPECT-1 or nave mice controls and incubated for 28-30 hours at 37° C. in 5% CO₂ incubator. In this experiment; two different serum concentrations were used 10% and 2%. After the incubation, the hepatocytes from each well were trypsinized and the emitted GFP signal from each well was measured by using the LSRII machine. Serum from mice vaccinated with PfSPECT-1 showed high level of hepatocyte infection blocking; 95% and 93% invasion blocking using 10% serum from Balb/c and CD-1 mice, respectively, and 87% and 74% invasion blocking using 2% serum from Balb/c and CD-1 mice, respectively. Using serum from Balb/c mice vaccinated against PfCSP in the same showed 99% and 81% hepatocyte invasion blocking with 10% and 2% serum, respectively (FIG. 34).

7.1 Summary and Overview

These data provide compelling evidence that SPECT-1 is a very promising and surprising vaccine candidate for the prevention of P. falciparum malaria. The results are especially surprising given the prior evidence that CS protein is the most abundant protein on the sporozoite surface and a very well studied protective antigen. Here we show that SPECT-1 can produce a protective immune responses that in outbred CD-1 mice exceeds substantially the efficacy achieved by equivalent CS-based vaccines. Efficacy on outbred mice is considered a particularly good indicator of likely efficacy in humans because of the genetic diversity of outbred mice. These findings provide the exciting opportunity of a SPECT-1 based vaccine that could outperform CS-based candidate vaccines or could be used to enhance the immunogenicity of existing CS-based malaria vaccines. The sporozoite invasion inhibition data suggests strongly that, like with CS-based vaccines, the mechanism of efficacy involves antibodies that prevent sporozoites invading hepatocytes. In contrast evaluation of 10 other antigens in this work failed to find evidence that these antigens could induce antibodies that protected against malaria. Vaccines based on the finding here of high level efficacy using the SPECT-1 antigen could comprise viral vectored vaccines, as used here, protein- or virus-like particle-based vaccines, DNA-based vaccines or a variety of other vaccine types well known in the art.

PfSPECT-1 sequences
A- PfSPECT-1 protein sequence with tPA leader
underlined
(SEQ ID NO: 12)
MKRGLCCVLLLCGAVFVSPSQEIHARFRRGMKMKIPICFLIILVLLKCVL
SYNLNNDLSKNNNFSLNTYVRKDDVEDDSKNEIVDNIQKMVDDFSDDIGF
VKTSMREVLLDTEASLEEVSDHVVQNISKYSLTIEEKLNLFDGLLEEFIE
NNKGLISNLSKRQQKLKGDKIKKVCDLILKKLKKLENVNKLIKYKIILKY
GNKDNKKEMIQTLKNEEGLSDDFKNNLSNYETEQNNDDIKEIELVNFIST
NYDKFVVNLEDLNKELLKDLNMALS
B- PfSPECT-1 protein sequence without leader
(SEQ ID NO: 13)
MKMKIPICFLIILVLLKCVLSYNLNNDLSKNNNFSLNTYVRKDDVEDDSK
NEIVDNIQKMVDDFSDDIGFVKTSMREVLLDTEASLEEVSDHVVQNISKY
SLTIEEKLNLFDGLLEEFIENNKGLISNLSKRQQKLKGDKIKKVCDLILK
KLKKLENVNKLIKYKIILKYGNKDNKKEMIQTLKNEEGLSDDFKNNLSNY
ETEQNNDDIKEIELVNFISTNYDKFVVNLEDLNKELLKDLNMALS
C- PfSPECT-1 nucleic acid sequence (Human
Optimized Sequence for the vaccine).
(SEQ ID NO: 14)
ATGAAGATGAAGATCCCTATCTGCTTCCTGATCATCCTGGTGCTGCTGAA
GTGCGTGCTGAGCTACAACCTGAACAACGACCTGAGCAAGAACAACAACT
TCAGCCTGAACACCTACGTGCGGAAGGACGACGTGGAAGATGACAGCAAG
AACGAGATCGTGGACAACATCCAGAAAATGGTGGACGACTTCAGCGACGA
CATCGGCTTCGTGAAAACCAGCATGAGAGAGGTGCTGCTGGACACCGAGG
CCAGCCTGGAAGAGGTGTCCGACCACGTGGTGCAGAACATCAGCAAGTAC
AGCCTGACCATCGAGGAAAAGCTGAACCTGTTCGACGGCCTGCTGGAAGA
GTTCATCGAGAACAACAAGGGCCTGATCAGCAACCTGTCCAAGCGGCAGC
AGAAGCTGAAGGGCGACAAGATCAAGAAAGTGTGCGACCTGATCCTGAAG
AAGCTGAAAAAGCTGGAAAACGTGAACAAGCTGATCAAGTACAAGATCAT
CCTGAAGTACGGCAACAAGGACAACAAGAAAGAGATGATCCAGACCCTGA
AGAACGAGGAAGGCCTGAGCGACGACTTCAAGAACAACCTGAGCAACTAC
GAGACAGAGCAGAACAACGACGACATCAAAGAAATCGAGCTGGTGAACTT
CATCTCCACCAACTACGACAAGTTCGTGGTGAACCTGGAAGATCTGAACA
AAGAGCTGCTGAAGGACCTGAACATGGCCCTGAGC
D- PfSPECT-1 wild-type gene nucleic coding
sequence (accession number: PF3D7_1342500)
(SEQ ID NO: 15)
ATGAAAATGAAAATCCCGATTTGTTTTCTCATTATTTTAGTCTTGTTAAA
ATGTGTGCTATCTTACAATCTAAATAACGACTTATCAAAAAATAATAATT
TTTCCTTAAATACATATGTCAGAAAAGATGATGTGGAAGATGATTCAAAA
AACGAGATTGTTGATAATATACAAAAAATGGTTGATGATTTTAGTGATGA
TATAGGTTTTGTAAAAACATCGATGCGTGAAGTTTTACTAGATACCGAAG
CGTCCCTTGAAGAAGTATCAGATCATGTTGTACAAAACATATCAAAATAT
AGTTTAACCATTGAAGAGAAACTTAATCTTTTTGATGGGCTTCTTGAAGA
ATTTATTGAAAATAATAAGGGCCTGATATCCAACTTATCAAAAAGACAAC
AAAAACTTAAGGGGGATAAAATTAAAAAGGTTTGTGATTTGATCTTAAAA
AAATTAAAAAAGTTAGAAAATGTCAACAAACTTATTAAATATAAGATAAT
ATTAAAATATGGAAATAAAGATAATAAAAAAGAAATGATACAAACATTGA
AAAATGAGGAGGGTTTATCTGATGACTTCAAAAATAATTTATCAAATTAT
GAAACAGAACAAAATAACGATGATATAAAAGAAATAGAATTAGTTAATTT
TATTTCAACAAATTATGATAAGTTTGTTGTTAATCTAGAAGACCTTAATA
AGGAGTTGCTAAAGGATTTAAACATGGCCTTATCATAA

### REFERENCES

1. Garcia, J. E., A. Puentes, and M. E. Patarroyo, Developmental biology of sporozoite-host interactions in Plasmodium falciparum malaria: implications for vaccine design. Clin Microbiol Rev, 2006. 19(4): p. 686-707.
2. Yuda, M. and T. Ishino, Liver invasion by malarial parasites—how do malarial parasites break through the host barrier? Cellular Microbiology, 2004. 6(12): p. 1119-1125.
3. Garcia, J. E., A. Puentes, and M. E. Patarroyo, Developmental biology of sporozoite-host interactions in Plasmodium falciparum malaria: Implications for vaccine design. Clinical Microbiology Reviews, 2006. 19(4): p. 686-+.
4. Kappe, S. H. I., C. A. Buscaglia, and V. Nussenzweig, Plasmodium sporozoite molecular cell biology. Annual Review of Cell and Developmental Biology, 2004. 20: p. 29-59.
5. Patarroyo, M. A., D. Calderon, and D. A. Moreno-Perez, Vaccines against Plasmodium vivax: a research challenge. Expert Rev Vaccines, 2012. 11(10): p. 1249-60.
6. Akhouri, R. R., et al., Role of Plasmodium falciparum thrombospondin-related anonymous protein in host-cell interactions. Malaria Journal, 2008. 7.
7. Ishino, T., Y. Chinzei, and M. Yuda, A Plasmodium sporozoite protein with a membrane attack complex domain is required for breaching the liver sinusoidal cell layer prior to hepatocyte infection. Cellular Microbiology, 2005. 7(2): p. 199-208.
8. Ishino, T., et al., Cell-passage activity is required for the malarial parasite to cross the liver sinusoidal cell layer. Plos Biology, 2004. 2(1): p. 77-84.
9. Ishino, T., Y. Chinzei, and M. Yuda, A Plasmodium sporozoite protein with a membrane attack complex domain is required for breaching the liver sinusoidal cell layer prior to hepatocyte infection. Cell Microbiol, 2005. 7(2): p. 199-208.
10. Yuda, M. and T. Ishino, Liver invasion by malarial parasites—how do malarial parasites break through the host barrier? Cell Microbiol, 2004. 6(12): p. 1119-25.
11. Lindner, S. E., et al., Total and putative surface proteomics of malaria parasite salivary gland sporozoites. Mol Cell Proteomics, 2013. 12(5): p. 1127-43.
12. Santos, J. M., et al., Apicomplexan cytoskeleton and motors: key regulators in morphogenesis, cell division, transport and motility. Int J Parasitol, 2009. 39(2): p. 153-62.
13. Amino, R., et al., Host cell traversal is important for progression of the malaria parasite through the dermis to the liver. Cell Host & Microbe, 2008. 3(2): p. 88-96.
14. Ishino, T., et al., Cell-passage activity is required for the malarial parasite to cross the liver sinusoidal cell layer. PLoS Biol, 2004. 2(1): p. E4.
15. Mota, M. M., et al., Migration of Plasmodium sporozoites through cells before infection. Science, 2001. 291(5501): p. 141-144.
16. Patarroyo, M. E., M. P. Alba, and H. Curtidor, Biological and structural characteristics of the binding peptides from the sporozoite proteins essential for cell traversal (SPECT)-1 and -2. Peptides, 2011. 32(1): p. 154-60.
17. Kappe, S. H., C. A. Buscaglia, and V. Nussenzweig, Plasmodium sporozoite molecular cell biology. Annu Rev Cell Dev Biol, 2004. 20: p. 29-59.
18. Frevert, U., et al., Intravital observation of Plasmodium berghei sporozoite infection of the liver. PLoS Biol, 2005. 3(6): p. e192.
19. Herrera, S., et al., Antigenicity and immunogenicity of multiple antigen peptides (MAP) containing P. vivax CS epitopes in Aotus monkeys. Parasite Immunol, 1997. 19(4): p. 161-70.
20. Frevert, U., et al., Intravital observation of Plasmodium berghei sporozoite infection of the liver. Plos Biology, 2005. 3(6): p. 1034-1046.
21. Baum, J., et al., Host-cell invasion by malaria parasites: insights from Plasmodium and Toxoplasma. Trends Parasitol, 2008. 24(12): p. 557-63.
22. Ejigiri, I. and P. Sinnis, Plasmodium sporozoite-host interactions from the dermis to the hepatocyte. Curr Opin Microbiol, 2009. 12(4): p. 401-7.
23. Ny, T., F. Elgh, and B. Lund, The structure of the human tissue-type plasminogen activator gene: correlation of intron and exon structures to functional and structural domains. Proc Natl Acad Sci USA, 1984. 81(17): p. 5355-9.
24. Becker, S. I., et al., Protection of mice against Plasmodium yoelii sporozoite challenge with P. yoelii merozoite surface protein 1 DNA vaccines. Infect Immun, 1998. 66(7): p. 3457-61.
25. Li, Z., et al., Immunogenicity of DNA vaccines expressing tuberculosis proteins fused to tissue plasminogen activator signal sequences. Infect Immun, 1999. 67(9): p. 4780-6.
26. Luo, M., et al., Immunization with plasmid DNA encoding influenza A virus nucleoprotein fused to a tissue plasminogen activator signal sequence elicits strong immune responses and protection against H5N1 challenge in mice. J Virol Methods, 2008. 154(1-2): p. 121-7.
27. Forbes, E. K., et al., Combining liver- and blood-stage malaria viral-vectored vaccines: investigating mechanisms of CD8+ T cell interference. J Immunol, 2011. 187(7): p. 3738-50.
28. Mueller, A. K., et al., Plasmodium liver stage developmental arrest by depletion of a protein at the parasite-host interface. Proc Natl Acad Sci USA, 2005. 102(8): p. 3022-7.
29. Silvie, O., et al., Post-transcriptional silencing of UIS4 in Plasmodium berghei sporozoites is important for host switch. Mol Microbiol, 2014. 91(6): p. 1200-13.

1. An antigenic composition or vaccine comprising a viral vector, the viral vector comprising nucleic acid encoding Plasmodium protein PfLSA1, or a part or variant of Plasmodium protein PfLSA1.

2. An antigenic composition or vaccine comprising a viral vector, the viral vector comprising nucleic acid encoding Plasmodium protein PfLSAP2, or a part or variant of Plasmodium protein PfLSAP2.

3. An antigenic composition or vaccine comprising a viral vector, the viral vector comprising nucleic acid encoding Plasmodium protein PfUIS3, or a part or variant of Plasmodium protein PfUIS3.

4. An antigenic composition or vaccine comprising a viral vector, the viral vector comprising nucleic acid encoding Plasmodium protein PfI0580c, or a part or variant of Plasmodium protein PfI0580c.

5. An antigenic composition or vaccine comprising a viral vector, the viral vector comprising nucleic acid encoding Plasmodium protein PfSPECT-1, or a part or variant of Plasmodium protein PfSPECT-1.

6. The antigenic composition or vaccine according to any preceding claim,
wherein the antigenic composition or vaccine is capable of eliciting a protective immune response against malaria in a subject.

7. The antigenic composition or vaccine according to claim 6, wherein a protective immune response comprises
at least 0.2% of CD8 cells being antigen-specific, and/or
at least 500 spot forming cells (SFU) per million peripheral blood mononuclear cells (PBMC) as determined by an ELISpot assay.

8. The antigenic composition or vaccine according to any of claims 1, 6 or 7,
wherein PfLSA1 comprises or consists of the sequence of SEQ ID NO: 1 or 2.

9. The antigenic composition or vaccine according to any of claims 2, 6 or 7,
wherein PfLSAP2 comprises or consists of the sequence of SEQ ID NO: 4 or 5.

10. The antigenic composition or vaccine according to any of claims 3, 6 or 7,
wherein PfUIS3 comprises or consists of the sequence of SEQ ID NO: 7.

11. The antigenic composition or vaccine according to any of claims 4, 6 or 7,
wherein PfI0580c comprises or consists of the sequence of SEQ ID NO: 9 or SEQ ID NO: 10.

12. The antigenic composition or vaccine according to any of claims 5-7,
wherein PfSPECT-1 comprises or consists of the sequence of SEQ ID NO: 12 or SEQ ID NO: 13.

13. The antigenic composition or vaccine according to any of claim 1, or 6-8,
wherein the nucleic acid encoding PfLSA1 comprises or consists of the sequence of SEQ ID NO: 3.

14. The antigenic composition or vaccine according to any of claims 2, 6, 7, or 9, wherein the nucleic acid encoding PfLSAP2 comprises or consists of the sequence of SEQ ID NO: 6.

15. The antigenic composition or vaccine according to any of claims 3, 6, 7, or 10, wherein the nucleic acid encoding PfUIS3 comprises or consists of the sequence of SEQ ID NO: 8.

16. The antigenic composition or vaccine according to any of claims 4, 6, 7, or 11, wherein the nucleic acid encoding PfI0580c comprises or consists of the sequence of SEQ ID NO: 11.

17. The antigenic composition or vaccine according to any of claims 5-7, or 12 wherein the nucleic acid encoding PfSPECT-1 comprises or consists of the sequence of SEQ ID NO: 14 or SEQ ID NO: 15.

18. The antigenic composition or vaccine according to any preceding claim,
wherein the variant Plasmodium protein comprises at least 50% amino acid sequence identity to SEQ ID NO: 1, 2, 4, 5, 7, 9, 10, 12 or 13.

19. The antigenic composition or vaccine according to any preceding claim,
wherein the viral vector comprises anadenovirus or poxvirus.

20. The antigenic composition or vaccine according to any preceding claim,
wherein the viral vector comprises a simian adenovirus such as ChAd63, ChAdOx1 or Modified Vaccinia Ankara (MVA) virus.

21. The antigenic composition or vaccine according to any preceding claim,
wherein the nucleic acid further encodes at least one other Plasmodium protein.

22. The antigenic composition or vaccine according to claim 21, wherein the at least one other Plasmodium protein selected from the group comprising PfLSA1, PfLSAP2, PfUIS3, PfI0580c, PfTRAP, PfCSP, PfSPECT-1, and a Plasmodium antigen capable of eliciting an immunogenic response in a subject, or combinations thereof.

23. The antigenic composition or vaccine according to any preceding claim,
wherein the Plasmodium comprises P. falciparum or P. vivax.

24. The antigenic composition or vaccine according to any preceding claim, further comprising an adjuvant.

25. The antigenic composition or vaccine according to any preceding claim,
wherein the malaria comprises liver-stage, or pre-liver stage, malaria.

26. A pharmaceutical composition comprising the immunogenic composition or vaccine according to any preceding claim and a pharmaceutically acceptable carrier.

27. The pharmaceutical composition according to claim 26, further comprising an adjuvant.

28. A nucleic acid encoding a viral protein and a Plasmodium protein, wherein the Plasmodium protein comprises PfLSA1, or a part or variant of PfLSA1, and
wherein the viral protein comprises an adenovirus protein or poxvirus protein.

29. A nucleic acid encoding a viral protein and a Plasmodium protein, wherein the Plasmodium protein comprises PfLSAP2, or a part or variant of PfLSAP2, and wherein the viral protein comprises an adenovirus protein or poxvirus protein.

30. A nucleic acid encoding a viral protein and a Plasmodium protein, wherein the Plasmodium protein comprises PfUIS3, or a part or variant of PfUIS3, and
wherein the viral protein comprises an adenovirus protein or poxvirus protein.

31. A nucleic acid encoding a viral protein and a Plasmodium protein, wherein the Plasmodium protein comprises PfI0580c, or a part or variant of PfI0580c, and wherein the viral protein comprises an adenovirus protein or poxvirus protein.

32. A nucleic acid encoding a viral protein and a Plasmodium protein, wherein the Plasmodium protein comprises PfSPECT-1, or a part or variant of PfSPECT-1, and wherein the viral protein comprises an adenovirus protein or poxvirus protein.

33. A nucleic acid encoding a viral protein and at least two Plasmodium proteins, wherein the at least two Plasmodium proteins are selected from any of the goup comprising PfLSA1 or a part or variant of PfLSA1; PfLSAP2 or a part or variant of PfLSAP2; PfUIS3 or a part or variant of PfUIS3; and PfI0580c or a part or variant of PfI0580c; PfSPECT-1 or a part or variant of PfSPECT-1; or combinations thereof, and wherein the viral protein comprises an adenovirus protein or poxvirus protein.

34. The nucleic acid according to any of claims 28-33, wherein the poxvirus protein comprises MVA protein.

35. A virus comprising the nucleic acid according to any of claims 28-34.

36. The virus according to claim 35, wherein the virus particle comprises a Plasmodium protein selected from the group comprising PfLSA1, or a part or variant of PfLSA1; PfLSAP2, or a part or variant of PfLSAP2; PfUIS3, or a part or variant of PfUIS3; and PfI0580c, or a part or variant of PfI0580c; PfSPECT-1 or a part or variant of PfSPECT-1; or combinations thereof.

37. The virus according to claim 35 or 36, wherein the virus is adenovirus or MVA.

38. The virus according to any of claims 35-37, wherein the virus is ChAd63, ChAdOx1 or MVA.

39. An in vitro host cell comprising the nucleic acid according to any of claims 38-34.

40. The host cell according to claim 35, wherein the host cell is infected with the virus of any of claims 35-38.

41. A method of eliciting a protective immune response to Plasmodium in a host, comprising administering the immunogenic composition or vaccine according to any of claims 1 to 25, or the pharmaceutical composition according to any of claims 26 or 27, to the host.

42. The method according to claim 41, wherein the protective immune response is a CD8+ T-cell response and/or a humoral response.

43. The method according to claim 42, wherein the protective immune response comprises:
at least 0.2% of CD8 cells being antigen-specific, and/or
at least 500 spot forming cells (SFU) per million peripheral blood mononuclear cells (PBMC) as determined by an ELISpot assay.

44. A method of prevention or treatment of malaria in a subject, comprising the administration of the immunogenic composition or vaccine according to any of claims 1 to 25, or the pharmaceutical composition according to any of claim 26 or 27.

45. The method according to any of claims 41-44, wherein the administration is part of a prime-boost vaccination regime in a subject, where a first/prime administration of the immunogenic composition or vaccine according to any of claims 1-25, or the pharmaceutical composition according to any of claim 26 or 27 is followed by a second/boost administration of the immunogenic composition or vaccine according to any of claims 1-25, or the pharmaceutical composition according to any of claims 26 or 27.

46. The method according to claim 45, wherein the viral vector of the first/prime administration comprises adenovirus.

47. The method according to claims 45 or 46, wherein the viral vector of the second/boost administration comprises poxvirus, such as MVA.

48. A method of prevention or treatment of malaria in a subject, comprising:
a first administration of the immunogenic composition or vaccine according to any of claims 1-25, or the pharmaceutical composition according to any of claims 26 or 27; and
a second administration of the immunogenic composition or vaccine according to any of claims 1-25, or the pharmaceutical composition according to any of claims 26 or 27.

49. The method according to claim 48, wherein the second administration is between about 10 days and about 30 days after the first administration.

50. The immunogenic composition or vaccine according to any of claims 1-25, or the pharmaceutical composition of claims 26 or 27, for use in prevention or treatment of malaria in a subject.

51. The immunogenic composition or vaccine for use according to claim 50,
wherein the use is in a prime-boost vaccination regime in the subject.

52. A kit for a vaccination regime against malaria in a subject, comprising:
a prime composition comprising a adenovirus comprising nucleic acid encoding Plasmodium protein PfLSA1, or a part or variant of Plasmodium protein PfLSA1; and/or
a boost composition comprising a MVA virus comprising nucleic acid encoding Plasmodium protein PfLSA1, or a part or variant of Plasmodium protein PfLSA1.

53. A kit for a vaccination regime against malaria in a subject, comprising:
a prime composition comprising a adenovirus comprising nucleic acid encoding Plasmodium protein PfLSAP2, or a part or variant of Plasmodium protein PfLSAP2; and/or
a boost composition comprising a MVA virus comprising nucleic acid encoding Plasmodium protein PfLSAP2, or a part or variant of Plasmodium protein PfLSAP2.

54. A kit for a vaccination regime against malaria in a subject, comprising:
a prime composition comprising a adenovirus comprising nucleic acid encoding Plasmodium protein PfUIS3, or a part or variant of Plasmodium protein PfUIS3; and/or
a boost composition comprising a MVA virus comprising nucleic acid encoding Plasmodium protein PfUIS3, or a part or variant of Plasmodium protein PfUIS3.

55. A kit for a vaccination regime against malaria in a subject, comprising:
a prime composition comprising a adenovirus comprising nucleic acid encoding Plasmodium protein PfI0580c, or a part or variant of Plasmodium protein PfI0580c; and/or
a boost composition comprising a MVA virus comprising nucleic acid encoding Plasmodium protein PfI0580c, or a part or variant of Plasmodium protein PfI0580c.

56. A kit for a vaccination regime against malaria in a subject, comprising:
a prime composition comprising a adenovirus comprising nucleic acid encoding Plasmodium protein PfSPECT-1, or a part or variant of Plasmodium protein PfSPECT-1; and/or
a boost composition comprising a MVA virus comprising nucleic acid encoding Plasmodium protein PfSPECT-1, or a part or variant of Plasmodium protein PfSPECT-1.

57. The kit according to any of claims 52-56, further comprising directions to administer the prime composition prior to the boost composition in a subject.

58. The kit according to any of claims 52-57, wherein the nucleic acid of the adenovirus and/or MVA virus further encodes a one or more other Plasmodium proteins.

59. The kit according to claim 58, wherein the one or more other Plasmodium proteins are Plasmodium antigens capable of eliciting an immune response in a subject.

60. The kit according to any of claims 52-59, wherein the prime and/or boost composition further comprises an adjuvant.

61. A method of manufacturing an immunogenic composition or vaccine according to claims 1-25, comprising:
culturing host cells capable of facilitating viral replication;
infecting the host cells with a virus according to claims 35-38, or transforming the cells with nucleic acid according to claims 28-34;
incubating the host cells to allow the production of viral progeny; and
harvesting the viral progeny to provide the immunogenic composition or vaccine.

62. The antigenic composition or vaccine according to any of claims 1-25, for use as a prime administration in a prime-boost vaccine regime; and/or for use as a boost administration in a prime-boost vaccine regime.


SEQUENCE LISTING (41 sequences)

SEQ ID NO: 1 - protein, 491 aa, Plasmodium falciparum
MKRGLCCVLLLCGAVFVSPSQEIHARFRRGMKHILYISFYFILVNLLIFHINGKIIKNSEKDEIIKSNLRSGSSNSRNRINEEKHEKKHVLSHNSYEKTKNNENNKFFDKDKELTMSNVKNVSQTNFKSLLRNLGVSENIFLKENKLNKEGKLIEHIINDDDDKKKYIKGQDENRQEDLEQERLAKEKLQEQQSDLERTKASTETLREQQSRKADTKKNLERKKEHGDVLAEDLYGRLEIPAIELPSENERGYYIPHQSSLPQDNRGNSRDSKEISIIENTNRESITTNVEGRRDIHKGHLEEKKDGSIKPEQKEDKSADIQNHTLETVNISDVNDFQISKYEDEISAEYDDSLIDEEEDDEDLDEFKPIVQYDNFQDEENIGIYKELEDLIEKNENLDDLDEGIEKSSEELSEEKIKKGKKYEKTKDNNFKPNDKSLYDEHIKKYKNDKQVNKEKEKFIKSLFHIFDGDNEILQIVDELSEDITKYFMKL

SEQ ID NO: 2 - protein, 460 aa, Plasmodium falciparum
KHILYISFYFILVNLLIFHINGKIIKNSEKDEIIKSNLRSGSSNSRNRINEEKHEKKHVLSHNSYEKTKNNENNKFFDKDKELTMSNVKNVSQTNFKSLLRNLGVSENIFLKENKLNKEGKLIEHIINDDDDKKKYIKGQDENRQEDLEQERLAKEKLQEQQSDLERTKASTETLREQQSRKADTKKNLERKKEHGDVLAEDLYGRLEIPAIELPSENERGYYIPHQSSLPQDNRGNSRDSKEISIIENTNRESITTNVEGRRDIHKGHLEEKKDGSIKPEQKEDKSADIQNHTLETVNISDVNDFQISKYEDEISAEYDDSLIDEEEDDEDLDEFKPIVQYDNFQDEENIGIYKELEDLIEKNENLDDLDEGIEKSSEELSEEKIKKGKKYEKTKDNNFKPNDKSLYDEHIKKYKNDKQVNKEKEKFIKSLFHIFDGDNEILQIVDELSEDITKYFMKL

SEQ ID NO: 3 - DNA, 1489 bp, Plasmodium falciparum
gtaccgccaccatgaagcggggcctgtgctgcgtgctgctgctgtgtggcgccgtgttcgtgtcccccagccaggaaatccacgcccggttcagacggggcatgaagcacatcctgtacatcagcttctacttcatcctggtgaacctgctgatcttccacatcaacggcaagatcatcaagaacagcgagaaggacgagatcattaagagcaacctgcggagcggcagcagcaacagccggaaccggatcaacgaggaaaagcacgagaagaaacacgtgctgagccacaacagctacgaaaagaccaagaacaatgagaacaacaagttcttcgacaaggacaaagaactgaccatgagcaacgtgaagaacgtgtcccagaccaacttcaagagcctgctgcggaacctgggcgtgtccgagaacatcttcctgaaagagaacaagctgaacaaagagggcaagctgatcgagcacatcatcaacgacgacgacgataagaagaagtacatcaagggccaggacgagaaccggcaggaagatctggaacaggaacggctggccaaagagaagctgcaggaacagcagagcgacctggaacggaccaaggccagcaccgagacactgagagagcagcagagcagaaaggccgacaccaagaagaacctggaacggaagaaagaacacggcgacgtgctggccgaggacctgtacggcagactggaaatccccgccatcgagctgcccagcgagaacgagcggggctactacatcccccaccagagcagcctgccccaggacaaccggggcaacagcagagacagcaaagagatcagcatcatcgagaacacaaaccgcgagagcatcaccaccaacgtggaaggcagacgggacatccacaagggccacctggaagagaagaaggacggcagcatcaagcccgagcagaaagaggacaagagcgccgacatccagaaccacaccctggaaaccgtgaacatcagcgacgtgaacgacttccagatctctaagtacgaggatgagatcagcgccgagtacgacgacagcctgatcgacgaggaagaggacgacgaggacctggacgagttcaagcccatcgtgcagtacgacaacttccaggacgaggaaaacatcggcatctacaaagagctggaagatctgatcgagaagaacgagaacctggatgatctggacgagggcatcgagaagtccagcgaggaactgagcgaggaaaagatcaagaagggcaagaagtacgagaaaactaaggacaacaacttcaagcccaacgacaagagcctgtacgatgagcacatcaagaagtataagaacgacaaacaggtgaacaaagagaaagagaagttcatcaagtccctgttccacatcttcgacggcgacaacgagatcctgcagatcgtggatgagctgtccgaggacatcaccaagtacttcatgaagctgtgagc

SEQ ID NO: 4 - protein, 332 aa, Plasmodium falciparum
MKRGLCCVLLLCGAVFVSPSQEIHARFRRGMWLCKRGLSVNDTTKCDVPCKDFYMLFLSNKKEKIKCGTFFGYIFLSKFMKLSISLLLLALIQNILLSNVSLISGSHLYKRNSRKFAEGYMKGSGSEKNVYLSNKNKEINMNQQSDNKMCDECDDMNQPGDVNKNDKTSNDQANSSDSDCEPLPFGLKPSDLNRKVTEEDLERMIIELPGKLERKDMYLIWHYSHSLLRDKFNKMKSSLWSICGKLAHEHKLPFKIKMKKWWKCCGHVTDELLIKEHDDYNSIYNYINNESSSREQFLIFLNMIKHSWTTFTMETFIKCKISLENNMRNVTN

SEQ ID NO: 5 - protein, 301 aa, Plasmodium falciparum
WLCKRGLSVNDTTKCDVPCKDFYMLFLSNKKEKIKCGTFFGYIFLSKFMKLSISLLLLALIQNILLSNVSLISGSHLYKRNSRKFAEGYMKGSGSEKNVYLSNKNKEINMNQQSDNKMCDECDDMNQPGDVNKNDKTSNDQANSSDSDCEPLPFGLKPSDLNRKVTEEDLERMIIELPGKLERKDMYLIWHYSHSLLRDKFNKMKSSLWSICGKLAHEHKLPFKIKMKKWWKCCGHVTDELLIKEHDDYNSIYNYINNESSSREQFLIFLNMIKHSWTTFTMETFIKCKISLENNMRNVTN

SEQ ID NO: 6 - DNA, 1012 bp, Plasmodium falciparum
gtaccgccaccatgaagcggggcctgtgctgcgtgctgctgctgtgtggcgccgtgttcgtgtcccccagccaggaaatccacgcccggttcagacggggcatgtggctgtgcaagcggggcctgagcgtgaacgacaccaccaagtgcgacgtgccctgcaaggacttctacatgctgtttctgagcaacaagaaagaaaagatcaagtgcggcaccttcttcggctacatcttcctgagcaagttcatgaagctgagcatcagcctgctgctgctggccctgatccagaacatcctgctgagcaacgtgtccctgatcagcggcagccacctgtacaagcggaacagccggaagttcgccgagggctacatgaagggcagcggctcagagaagaacgtgtacctgtccaacaagaacaaagaaatcaacatgaaccagcagagcgacaacaagatgtgcgacgagtgtgacgacatgaatcagcccggcgacgtgaacaagaacgacaagaccagcaacgaccaggccaacagcagcgacagcgactgcgagcccctgcccttcggcctgaagcccagcgacctgaaccggaaagtgaccgaagaggacctggaacggatgatcatcgagctgcccggcaagctggaacggaaggacatgtacctgatctggcactacagccacagcctgctgagagacaagttcaacaagatgaagtccagcctgtggtccatctgtggcaagctggcccacgagcacaagctgcccttcaagatcaagatgaagaaatggtggaagtgctgcggccacgtgaccgacgagctgctgatcaaagagcacgacgactacaacagcatctacaactacatcaacaacgagtctagcagccgcgagcagttcctgattttcctgaacatgatcaagcacagctggaccaccttcaccatggaaaccttcatcaagtgcaagatcagcctggaaaacaacatgcggaacgtgaccaactgagc

SEQ ID NO: 7 - protein, 229 aa, Plasmodium falciparum
MKVSKLVLFAHIFFIINILCQYICLNASKVNKKGKIAEEKKRKNIKNIDKAIEEHNKRKKLIYYSLIASGAIASVAAILGLGYYGYKKSREDDLYYNKYLEYRNGEYNIKYQDGAIASTSEFYIEPEGINKINLNKPIIENKNNVDVSIKRYNNFVDIARLSIQKHFEHLSNDQKDSHVNNMEYMQKFVQGLQENRNISLSKYQENKAVMDLKYHLQKVYANYLSQEEN

SEQ ID NO: 8 - DNA, 703 bp, Plasmodium falciparum
gtaccgccaccatgaaggtgtccaagctggtgctgttcgcccacatctttttcatcatcaacatcctgtgccagtacatctgcctgaacgccagcaaagtgaacaagaagggcaagatcgccgaagagaagaaaagaaagaacatcaagaatatcgacaaggccatcgaggaacacaacaagcggaagaagctgatctactacagcctgatcgctagcggcgccattgcctctgtggccgctatcctgggcctgggctactacggctacaagaaaagcagagaggacgacctgtactacaacaagtacctggaataccggaacggcgagtacaacatcaagtaccaggacggcgctatcgccagcaccagcgagttctacatcgagcccgagggcatcaacaagatcaacctgaacaagcccatcatcgagaacaagaacaacgtggacgtgtccatcaagcggtacaacaacttcgtggatatcgcccggctgagcatccagaagcacttcgagcacctgagcaacgaccagaaagacagccacgtgaacaacatggagtacatgcagaaattcgtccagggcctgcaggaaaaccggaacatcagcctgagcaagtatcaggaaaacaaggccgtgatggacctgaagtaccatctgcagaaggtgtacgccaactacctgagccaggaagagaactgagc

SEQ ID NO: 9 - protein, 443 aa, Plasmodium falciparum
MKRGLCCVLLLCGAVFVSPSQEIHARFRRGMNLLVFFCFFLLSCIVHLSRCSDNNSYSFEIVNRSTWLNIAERIFKGNAPFNFTIIPYNYVNNSTEENNNKDSVLLISKNLKNSSNPVDENNHIIDSTKKNTSNNNNNNSNIVGIYESQVHEEKIKEDNTRQDNINKKENEIINNNHQIPVSNIFSENIDNNKNYIESNYKSTYNNNPELIHSTDFIGSNNNHTFNFLSRYNNSVLNNMQGNTKVPGNVPELKARIFSEEENTEVESAENNHTNSLNPNESCDQIIKLGDIINSVNEKIISINSTVNNVLCINLDSVNGNGFVWTLLGVHKKKPLIDPSNFPTKRVTQSYVSPDISVTNPVPIPKNSNTNKDDSINNKQDGSQNNTTTNHFPKPREQLVGGSSMLISKIKPHKPGKYFIVYSYYRPFDPTRDTNTRIVELNVQ

SEQ ID NO: 10 - protein, 412 aa, Plasmodium falciparum
NLLVFFCFFLLSCIVHLSRCSDNNSYSFEIVNRSTWLNIAERIFKGNAPFNFTIIPYNYVNNSTEENNNKDSVLLISKNLKNSSNPVDENNHIIDSTKKNTSNNNNNNSNIVGIYESQVHEEKIKEDNTRQDNINKKENEIINNNHQIPVSNIFSENIDNNKNYIESNYKSTYNNNPELIHSTDFIGSNNNHTFNFLSRYNNSVLNNMQGNTKVPGNVPELKARIFSEEENTEVESAENNHTNSLNPNESCDQIIKLGDIINSVNEKIISINSTVNNVLCINLDSVNGNGFVWTLLGVHKKKPLIDPSNFPTKRVTQSYVSPDISVTNPVPIPKNSNTNKDDSINNKQDGSQNNTTTNHFPKPREQLVGGSSMLISKIKPHKPGKYFIVYSYYRPFDPTRDTNTRIVELNVQ

SEQ ID NO: 11 - DNA, 1345 bp, Plasmodium falciparum
gtaccgccaccatgaagcggggcctgtgctgcgtgctgctgctgtgtggcgccgtgttcgtgtcccccagccaggaaatccacgcccggttcagacggggcatgaacctgctggtgttcttctgcttcttcctgctgtcctgcatcgtgcacctgagccggtgcagcgacaacaacagctacagcttcgagatcgtgaaccggtccacctggctgaatatcgccgagcggatcttcaagggcaacgcccccttcaacttcaccatcatcccttacaactacgtgaacaacagcaccgaggaaaacaacaacaaggactccgtgctgctgatctccaagaacctgaagaacagcagcaaccccgtggacgagaacaaccacatcatcgacagcaccaagaagaacacctccaacaacaataacaacaactccaacatcgtgggcatctacgagagccaggtgcacgaggaaaagatcaaagaggacaacacccggcaggacaacatcaacaagaaagagaacgagatcatcaacaacaaccaccagatccccgtgtccaacatcttcagcgagaacatcgataacaacaagaactacatcgagagcaactacaagagcacatacaacaacaatcccgagctgatccacagcaccgacttcatcggctctaacaacaatcacaccttcaactttctgagccggtacaacaatagcgtgctgaacaacatgcagggcaacaccaaggtgcccggcaacgtgcccgagctgaaggcccggatcttctccgaggaagagaacaccgaggtcgaaagcgccgaaaacaaccacaccaacagcctgaaccccaacgagagctgcgaccagatcatcaagctgggcgacatcatcaacagcgtgaacgagaagatcatcagcatcaactccaccgtgaacaacgtgctgtgcatcaacctggactccgtgaacggcaacggcttcgtgtggaccctgctgggcgtgcacaagaagaagcccctgatcgaccccagcaacttccccaccaagagagtgacccagagctacgtgtcccccgacatcagcgtgaccaaccccgtgcccatccccaagaacagcaacaccaacaaggatgacagcattaacaacaagcaggacggcagccagaacaacaccaccaccaaccacttccccaagccccgcgagcagctggtgggaggcagcagcatgctgattagcaagatcaagccccacaagcccggcaagtacttcatcgtgtacagctactaccggcccttcgaccccacccgggacaccaacacccggatcgtggaactgaacgtgcagtgagc

SEQ ID NO: 12 - protein, 275 aa, Plasmodium falciparum
MKRGLCCVLLLCGAVFVSPSQEIHARFRRGMKMKIPICFLIILVLLKCVLSYNLNNDLSKNNNFSLNTYVRKDDVEDDSKNEIVDNIQKMVDDFSDDIGFVKTSMREVLLDTEASLEEVSDHVVQNISKYSLTIEEKLNLFDGLLEEFIENNKGLISNLSKRQQKLKGDKIKKVCDLILKKLKKLENVNKLIKYKIILKYGNKDNKKEMIQTLKNEEGLSDDFKNNLSNYETEQNNDDIKEIELVNFISTNYDKFVVNLEDLNKELLKDLNMALS

SEQ ID NO: 13 - protein, 245 aa, Plasmodium falciparum
MKMKIPICFLIILVLLKCVLSYNLNNDLSKNNNFSLNTYVRKDDVEDDSKNEIVDNIQKMVDDFSDDIGFVKTSMREVLLDTEASLEEVSDHVVQNISKYSLTIEEKLNLFDGLLEEFIENNKGLISNLSKRQQKLKGDKIKKVCDLILKKLKKLENVNKLIKYKIILKYGNKDNKKEMIQTLKNEEGLSDDFKNNLSNYETEQNNDDIKEIELVNFISTNYDKFVVNLEDLNKELLKDLNMALS

SEQ ID NO: 14 - DNA, 735 bp, Plasmodium falciparum
atgaagatgaagatccctatctgcttcctgatcatcctggtgctgctgaagtgcgtgctgagctacaacctgaacaacgacctgagcaagaacaacaacttcagcctgaacacctacgtgcggaaggacgacgtggaagatgacagcaagaacgagatcgtggacaacatccagaaaatggtggacgacttcagcgacgacatcggcttcgtgaaaaccagcatgagagaggtgctgctggacaccgaggccagcctggaagaggtgtccgaccacgtggtgcagaacatcagcaagtacagcctgaccatcgaggaaaagctgaacctgttcgacggcctgctggaagagttcatcgagaacaacaagggcctgatcagcaacctgtccaagcggcagcagaagctgaagggcgacaagatcaagaaagtgtgcgacctgatcctgaagaagctgaaaaagctggaaaacgtgaacaagctgatcaagtacaagatcatcctgaagtacggcaacaaggacaacaagaaagagatgatccagaccctgaagaacgaggaaggcctgagcgacgacttcaagaacaacctgagcaactacgagacagagcagaacaacgacgacatcaaagaaatcgagctggtgaacttcatctccaccaactacgacaagttcgtggtgaacctggaagatctgaacaaagagctgctgaaggacctgaacatggccctgagc

SEQ ID NO: 15 - DNA, 738 bp, Plasmodium falciparum
atgaaaatgaaaatcccgatttgttttctcattattttagtcttgttaaaatgtgtgctatcttacaatctaaataacgacttatcaaaaaataataatttttccttaaatacatatgtcagaaaagatgatgtggaagatgattcaaaaaacgagattgttgataatatacaaaaaatggttgatgattttagtgatgatataggttttgtaaaaacatcgatgcgtgaagttttactagataccgaagcgtcccttgaagaagtatcagatcatgttgtacaaaacatatcaaaatatagtttaaccattgaagagaaacttaatctttttgatgggcttcttgaagaatttattgaaaataataagggcctgatatccaacttatcaaaaagacaacaaaaacttaagggggataaaattaaaaaggtttgtgatttgatcttaaaaaaattaaaaaagttagaaaatgtcaacaaacttattaaatataagataatattaaaatatggaaataaagataataaaaaagaaatgatacaaacattgaaaaatgaggagggtttatctgatgacttcaaaaataatttatcaaattatgaaacagaacaaaataacgatgatataaaagaaatagaattagttaattttatttcaacaaattatgataagtttgttgttaatctagaagaccttaataaggagttgctaaaggatttaaacatggccttatcataa

SEQ ID NO: 16 - DNA, 42 bp, Artificial Sequence
  Forward Primer
gccaacatgaagcttatgaacgccctgcggcggctgcctgtg

SEQ ID NO: 17 - DNA, 47 bp, Artificial Sequence
  reverse primer
cccgggcccggatccgtcgaagaaatcgtcgctcaggctttcctcgc

SEQ ID NO: 18 - DNA, 39 bp, Artificial Sequence
  forward primer
gccaacatgaagcttatgcggttcagcaaggtgttcagc

SEQ ID NO: 19 - DNA, 43 bp, Artificial Sequence
  reverse primer
cccgggcccggatccctgctctttcttgggttcctcggttttc

SEQ ID NO: 20 - DNA, 48 bp, Artificial Sequence
  forward primer
gccaacatgaagcttatgaagatcctgtccgtgttctttctggccctg

SEQ ID NO: 21 - DNA, 43 bp, Artificial Sequence
  reverse primer
cccgggcccggatccgtgctcggtgccggacaccaggttgttg

SEQ ID NO: 22 - DNA, 39 bp, Artificial Sequence
  forward primer
gccaacatgaagcttatgaacctgctggtgttcttctgc

SEQ ID NO: 23 - DNA, 39 bp, Artificial Sequence
  reverse primer
cccgggcccggatccctgcacgttcagttccacgatccg

SEQ ID NO: 24 - DNA, 48 bp, Artificial Sequence
  forward primer
gccaacatgaagcttatgaagcacatcctgtacatcagcttctacttc

SEQ ID NO: 25 - DNA, 43 bp, Artificial Sequence
  reverse primer
cccgggcccggatcccagcttcatgaagtacttggtgatgtcc

SEQ ID NO: 26 - DNA, 48 bp, Artificial Sequence
  forward primer
gccaacatgaagcttatgaccaacagcaactacaagagcaacaacaag

SEQ ID NO: 27 - DNA, 46 bp, Artificial Sequence
  reverse primer
cccgggcccggatcctttgcttttctgtgtccggctcttttttggc

SEQ ID NO: 28 - DNA, 40 bp, Artificial Sequence
  forward primer
gccaacatgaagcttatgaagaccatcatcatcgtgaccc

SEQ ID NO: 29 - DNA, 40 bp, Artificial Sequence
  reverse primer
cccgggcccggatccttccaccatgtagaagtcggcgtcc

SEQ ID NO: 30 - DNA, 39 bp, Artificial Sequence
  forward primer
gccaacatgaagcttatgtggctgtgcaagcggggcctg

SEQ ID NO: 31 - DNA, 43 bp, Artificial Sequence
  Reverse Primer
cccgggcccggatccgttggtcacgttccgcatgttgttttcc

SEQ ID NO: 32 - DNA, 43 bp, Artificial Sequence
  forward primer
gccaacatgaagcttatgaaggtgtccaagctggtgctgttcg

SEQ ID NO: 33 - DNA, 43 bp, Artificial Sequence
  Reverse Primer
cccgggcccggatccgttctcttcctggctcaggtagttggcg

SEQ ID NO: 34 - DNA, 20 bp, Artificial Sequence
  Forward primer
gcggagaatccgagatatga

SEQ ID NO: 35 - DNA, 20 bp, Artificial Sequence
  reverse primer
ccgcgctctggttgtagtag

SEQ ID NO: 36 - DNA, 20 bp, Artificial Sequence
  forward primer
accgtccagaggatgtatgg

SEQ ID NO: 37 - DNA, 20 bp, Artificial Sequence
  reverse primer
ccaggtaggctctcaactgc

SEQ ID NO: 38 - DNA, 20 bp, Artificial Sequence
  forward primer
tggcacctgctgagatactg

SEQ ID NO: 39 - DNA, 20 bp, Artificial Sequence
  reverse primer
cagttcctttgccctctctg

SEQ ID NO: 40 - DNA, 20 bp, Artificial Sequence
  forward primer
cttggacccttggtacctca

SEQ ID NO: 41 - DNA, 20 bp, Artificial Sequence
  reverse primer
aagtccagtgttgggtcagg